(19) United States
(12) Patent Application Publication
(10) Pub. No.: US 2004/0236091 A1
(43) Pub. Date: Nov. 25, 2004

(54) Translational profiling

(76) Inventors: Roman M. Chicz, Belmont, MA (US); Andrew J. Tomlinson, Wayland, MA (US); Robert G. Urban, Lexington, MA (US)

Correspondence Address: FISH & RICHARDSON PC, 225 FRANKLIN ST, BOSTON, MA, 02110, US

(21) Appl. No.: 10/473,127

(22) PCT Filed: Mar. 28, 2002
(86) PCT No.: PCT/US02/09671

(30) Foreign Application Priority Data
Mar. 28, 2001 (US) 60279495
May 21, 2001 (US) 60292544
Aug. 8, 2001 (US) 60310801
Oct. 1, 2001 (US) 60326370
Dec. 4, 2001 (US) 60336780
Feb. 20, 2002 (US) 60358985

(51) Int. Cl. C12Q 1/68; C07H 21/04

(52) U.S. Cl. 536/23.5; 435/6

(57) ABSTRACT

Polypeptides representative of proteins expressed by a given cell type and isolated nucleic acids that encode the polypeptides are disclosed. The compositions and method described can be used to define a cell type at a given developmental, metabolic, or disease stage by identifying and cataloging proteins expressed in the cell. The compositions can also be used in the manufacture of therapeutics as well as in diagnostics and drug screening.

### CROSS REFERENCE TO RELATED APPLICATIONS

This application claims priority from U.S. Provisional Application No. 60/279,495, filed Mar. 28, 2001, U.S. Provisional Application No. 60/292,544, filed May 21, 2001, U.S. Provisional Application No. 60/310,801, filed Aug. 8, 2001, U.S. Provisional Application No. 60/326,370, filed Oct. 1, 2001, U.S. Provisional Application No. 60/336,780, filed Dec. 4, 2001, and U.S. Provisional Application No. 60/358,985, filed Feb. 20, 2002. These applications are incorporated herein by reference in their entirety.

### FIELD OF THE INVENTION

The invention relates to peptides identified by translational profiling methods, as well as nucleic acids encoding the peptides, methods of using the peptides to characterize the protein composition of a cell, and methods of using the peptides to diagnose, prevent, and treat disease.

### REFERENCE TO SEQUENCE LISTING SUBMITTED ON A COMPACT DISC

This application includes a compact disc (four copies of disc submitted) containing a sequence listing. The sequence listing is identified on the compact disc as follows.

| File Name | Date of Creation | Size (bytes) |
|---|---|---|
| 08191-026WO1.TXT | Mar. 25, 2002 | 8,015,000 |

The entire content of the sequence listing is herein incorporated by reference.

### BACKGROUND OF THE INVENTION

Essentially every cell within an organism contains the complete and identical genetic information of that organism, but expresses only a subset of that total complement of genes. For example, the human genome, which is composed of a total of three billion nucleotides, is currently thought to include approximately 30,000-40,000 genes. However, individual cells expresses only about 2,000 to about 4,000 different proteins, corresponding to only 10% of the total number of genes. It is the concerted activity of the proteins expressed in a given cell that orchestrates the activities that define a particular cell type at a given developmental, metabolic or disease stage.

In the past decades it has become clear that the development and the pathology of many diseases involves differences in gene expression. Indeed, healthy and diseased tissue or cell types can frequently be distinguished by differences in gene expression. For example, normal cells may evolve to highly invasive and metastatic cancer cells by activation of certain growth-inducing genes, e.g., oncogenes, or the inactivation of certain growth-inhibitory genes, e.g., tumor suppressors or apoptosis activators. Levine, 1997, Cell 88:323; Hunter, 1997, Cell 88:333; Jacobson, 1997, Cell 88:347; Nagata, 1997, Cell 88:355; Fraser et al., 1996, Cell 85:781. Altered expression of such genes, e.g., growth activators or growth suppressors, in turn affects expression of other genes. See, The National Cancer Institute, “The Nation's Investment In Cancer Research: A Budget Proposal For Fiscal Years 1997/98”, Prepared by the Director, National Cancer Institute, pp. 55-77.

Pathological gene expression differences are not confined to cancer. Autoimmune disorders, many neurodegenerative diseases, inflammatory diseases, restenosis, atherosclerosis, many metabolic diseases, and numerous other disorders are believed to involve aberrant expression of particular genes. Naparstek et al., 1993, Ann. Rev. Immunol. 11:79; Sercarz et al., 1993, Ann. Rev. Immunol. 11:729. As a consequence, a challenge in medical research is to understand the role each gene or its encoded protein plays in maintaining normal cellular homeostasis and to utilize this heightened understanding in improving the ability to treat disease and/or identify predispositions to disease at stages when treatment and/or prevention methods are available.

Significant resources have been expended to identify and isolate genes relevant to disease development. One approach has been to sequence and catalogue all the individual genes contained in the genome of a species. In the case of humans, the NIH initiated the Humane Genome Project in 1990, with the goal to sequence the entire human genome by the year 2005. Stephens et al., 1990, Science 250:237; Cantor, 1990, Science 248:49-51. The near complete sequence of the human genome was published in advance of the 2005 target date. Venter et al., Science 2001 291:1304; International Human Genome Sequencing Consortium Nature 2001 409:860. However, the vast amount of information made available by the sequencing of the human genome is insufficient to resolve the mysteries of many disease processes because cellular function and dysfunction results from the concerted interaction and differential expression of proteins. Indeed, nucleotide sequence information alone does not indicate when, where, and how much of a given gene is expressed at the protein level.

### SUMMARY OF THE INVENTION

The present invention is based on the purification of a series of peptide sequences derived from proteins produced within a panel of cells. The purification and sequencing of these peptides demonstrates both the existence of a given protein as well as the production of the given protein in a particular cell type. In many cases, the existence of a given protein was uncertain prior to the characterization describe herein, as it had never previously been isolated or even detected. Members of one class of peptides described herein, termed expressed protein tags (EPTs), bind to and are presented by human MHC class I or class II molecules. Members of a second class of peptides are chemically or enzymatically prepared from complex protein mixtures.

The invention generally relates to novel peptides and proteins containing the novel amino acid sequences. In addition, the invention relates to nucleic acids encoding polypeptides containing the novel peptides, methods of using the peptide sequences in the context of a database or a peptide profile to characterize the protein composition of a cell or a peptide array comprising peptides of the invention, and using the identified peptides and corresponding nucleic acids in methods of treatment, diagnosis, and screening.

In one aspect, the invention features a purified polypeptide including a peptide sequence selected from the group consisting of SEQ ID NOs: 1-235. In an embodiment, the polypeptide comprises at least 8 contiguous amino acids of an amino acid sequence selected from the group consisting of SEQ ID NOs: 1-235. In another embodiment, the invention features a purified immunogenic polypeptide comprising at least 8 contiguous amino acids of an amino acid sequence selected from the group consisting of SEQ ID NOs: 1-235. “Immunogenic peptides” are peptides that result in or enhance an immune response in a mammal. Examples of immunogenic peptides can be found, for example in U.S. Pat. No. 5,827,516 and U.S. Pat. No. 6,183,746. In another embodiment, the invention features a purified polypeptide, comprising at least an immunogenic portion of a protein, wherein the protein comprises an amino acid sequence selected from the group consisting of SEQ ID NOs: 1-235.

In another aspect, the invention features a purified polypeptide comprising an amino acid sequence selected from the group consisting of SEQ ID NOs: 1-235, wherein the purified polypeptide comprises at least 25 amino acids. In an example, the purified polypeptide comprises fewer than 100 amino acids. In another example, the purified polypeptide comprises fewer than 50 amino acids.

In one embodiment, the polypeptide consists of a peptide sequence selected from the group consisting of SEQ ID NOs: 1-235. In another embodiment, the polypeptide consists essentially of an amino acid sequence selected from the group consisting of SEQ ID NOs: 1-235.

The peptide sequence can be identical to that of a naturally processed class I MHC-binding peptide. Alternatively, the peptide sequence can be identical to that of a naturally processed class II MHC-binding peptide.

In another aspect, the invention features an isolated nucleic acid encoding a polypeptide comprising a peptide sequence selected from the group consisting of SEQ ID NOs:1-235. In an embodiment, the polypeptide comprises an amino acid sequence which is at least 95% identical to an amino acid selected from the group consisting of SEQ ID NOs: 1-235. In another embodiment, the isolated nucleic acid comprises a nucleotide sequence that encodes a polypeptide comprising an amino acid sequence selected from the group consisting of a variant of any one of SEQ ID NOs: 1-235, wherein the variant has no more than two conservative amino acid substitutions. In a further embodiment, the isolated nucleic acid comprises a nucleotide sequence that encodes a polypeptide comprising at least 8 contiguous amino acids of an amino acid sequence selected from the group consisting of SEQ ID NOs: 1-235.

In some examples the encoded polypeptide includes a peptide sequence identical to that of a naturally processed class I MHC-binding peptide. Alternatively, the peptide sequence can be identical to that of a naturally processed class II MHC-binding peptide.

In one embodiment, an isolated nucleic acid encodes a polypeptide including a peptide sequence identical to a segment of a naturally occurring protein, wherein the peptide sequence is selected from the group consisting of SEQ ID NOs: 1-235, and wherein the polypeptide does not include more than 10, 15, 20, 30, 40, 50, 60, 70, 80, 90, or 100 consecutive amino acids identical to a portion of the naturally occurring protein. The peptide sequence can be identical to that of a naturally processed class I MHC-binding peptide. Alternatively, the peptide sequence can be identical to that of a naturally processed class II MHC-binding peptide.

In an aspect, the invention features an isolated nucleic acid comprising a nucleotide sequence encoding a polypeptide consisting of an amino acid sequence selected from the group consisting of SEQ ID NOs: 1-235. In another aspect, the isolated nucleic acid comprises a nucleotide sequence encoding a polypeptide consisting essentially of an amino acid sequence selected from the group consisting of SEQ ID NOs: 1-235.

The invention also includes an expression vector containing a nucleic acid described herein. In an example, the vector comprises expression control sequences that direct expression of the polypeptide. In another example, the vector comprises expression control sequences that direct expression of the nucleic acid molecule. Also included in the invention is a cell containing an expression vector of the invention.

In another aspect, the invention features an antibody specific for a polypeptide of the invention, e.g., a peptide sequence selected from the group consisting of SEQ ID NOs: 1-235. In an example, the antibody selectively binds to the polypeptide which is expressed on a cell surface. In another example, the antibody of the polypeptide is a target of a second antibody located on a cell surface.

In another aspect, the invention features a humanized antibody which specifically binds to a domain of a polypeptide comprising an amino acid sequence selected from the group consisting of SEQ ID NOs: 1-235 or an isolated nucleic acid which encodes the antibody. In preferred embodiments, the humanized antibody is a full length antibody, a human IgG, an antibody fragment and a F(ab)₂. The invention also features a humanized antibody as described herein bound to a detectable label. In another aspect, the invention features an immobilized antibody comprising a humanized antibody as described herein bound to a solid phase. In a further aspect, the invention features a conjugate comprising a humanized antibody as described herein bound to a cytotoxic agent.

The invention also includes a method for determining the presence of a protein comprising exposing a sample suspected of containing the protein to a humanized antibody as described herein and determining binding of the antibody to the sample. In another aspect, the invention includes a kit comprising a humanized antibody as described herein and instructions for using the humanized antibody to detect a protein that binds to the antibody.

The invention also includes a method of making an antibody, the method comprising: (a) providing a polypeptide comprising an amino acid sequence selected from the group consisting of SEQ ID NOs: 1-235 or a nucleic acid encoding such a polypeptide to a mammal in an amount effective to induce the production of an antibody that binds to the polypeptide; (b) isolating from the mammal a cell that produces an antibody that selectively binds to a polypeptide consisting of an amino acid sequence selected from the group consisting of SEQ ID NOs: 1-235; (c) immortalizing the cell isolated in step (b); and (d) isolating antibodies from the immortalized cell. The invention also includes a method of modulating the activity of a polypeptide described herein, the method including contacting the polypeptide with a compound that binds to the polypeptide in a concentration sufficient to modulate the activity of the polypeptide. In an example, the compound that binds the polypeptide is an antibody that selectively binds a polypeptide consisting of an amino acid sequence selected for the group consisting of SEQ ID NOs:1-235.

In another aspect, the invention features a method of treating a disorder in a mammal, the method including: (1) identifying a mammal with the disorder; and (2) administering to the mammal a compound that modulates the expression or activity of a polypeptide described herein, wherein the administration results in an amelioration of one or more symptoms of the disorder. The disorder can be for example a cellular proliferative and/or differentiative disorder or a disorder associated with the particular biological class of proteins to which the polypeptide belongs.

In another aspect, the invention features a method for detecting the presence of a polypeptide described herein in a sample, the method including: (1) contacting the sample with a compound that selectively binds to the polypeptide; and (2) determining whether the compound binds to the polypeptide in the sample.

In another aspect, the invention features a method for detecting the presence of a disorder in a mammal, the method including: (1) providing a biological sample derived from the mammal; (2) contacting the sample with a compound that binds to a polypeptide described herein or to a nucleic acid that encodes such a polypeptide; and (3) determining whether the compound binds to the sample, wherein binding of the compound to the sample indicates the presence or absence of the disorder in the mammal.

In another aspect, the invention features a method for imaging a site in a mammal, the method including: (1) administering a compound to a mammal, wherein the compound binds to a polypeptide described herein (or to a nucleic acid that encodes such a polypeptide) at the site in the mammal; and (2) detecting the compound with an imaging detector, to thereby image the site in the mammal.

In another aspect, the invention features a method for identifying a compound that modulates the activity of a polypeptide described herein, the method including: (1) contacting a polypeptide described herein with a test compound; and (2) determining the effect of the test compound on the activity of the polypeptide, to thereby identify a compound that modulates the activity of the polypeptide.

In another aspect, the invention features a method for identifying a compound that modulates the expression of a nucleic acid described herein, the method including: (1) contacting the nucleic acid with a test compound; and (2) determining the effect of the test compound on the expression of the nucleic acid, to thereby identify a compound that modulates the expression of the nucleic acid.

In another aspect, the invention features a peptide profile that is characteristic for a given cell, wherein the profile includes a representation of at least ten different polypeptides in the cell, wherein each of the at least ten different polypeptides contains a peptide selected from the group consisting of SEQ ID NOs: 1-235, and wherein the peptide profile is a reproducible characteristic of the cell. In one example, the each of the at least ten different polypeptides contains an MHC-binding peptide. In one example, the representation characterizes each individual peptide based upon at least one physical or chemical attribute, the at least one physical or chemical attribute including amino acid sequence. In addition, the representation can characterize each individual peptide based upon at least two physical or chemical attributes, e.g., wherein one of the physical or chemical attributes is amino acid sequence. For example, one of the physical or chemical attributes can be mass-to-charge ratio or ion-fragmentation pattern. In another example, the representation can characterize each individual peptide based upon at least three physical or chemical attributes. In another aspect, the invention features a polypeptide profile that is characteristic of a selected cell under selected conditions, wherein the profile comprises a representation of at least ten different polypeptides expressed by the cell, wherein each of the at least ten different polypeptides comprises an amino acid sequence selected from the group consisting of SEQ ID NOs: 1-235, and wherein the polypeptide profile is a reproducible characteristic of the cell.

In another aspect, the invention features a database, stored on a machine-readable medium, containing: two categories of data respectively representing (a) peptide profiles and (b) cell sources; and associations among instances of the two categories of data, wherein the data representing peptide profiles include a peptide profile described herein, and wherein the database configures a computer to enable finding instances of data of one of the categories based on their associations with instances of data the other category.

In another aspect, the invention features a database, stored on a machine-readable medium, comprising: (a) three categories of data respectively representing (i) polypeptides, (ii) cell sources, and (iii) cell treatments; and (b) associations among instances of the three categories of data, wherein the data representing peptides comprises at least 100 polypeptides each having an amino acid sequence selected from the group consisting of SEQ ID NOs: 1-235, and wherein the database configures a computer to enable finding instances of data of one of the categories based on their associations with instances of data of at least one other category.

In another aspect, the invention features a peptide array comprising at least 100 peptides selected from the group consisting of peptides consisting of an amino acid sequence selected from the group consisting of SEQ ID NOs:1-235, each peptide linked to a solid support at a known location. In another aspect the invention features a collection of at least 10 polypeptide arrays, each array comprising at least 100 polypeptides consisting of an amino acid sequence selected from the group consisting of SEQ ID NOs: 1-235, each peptide linked to a solid support at a known location.

In another aspect, the invention features a method of selecting an antibody, the method including: (1) contacting a polypeptide described herein with an in vitro library of antibodies; (2) binding an antibody to the polypeptide; and (3) selecting the antibody that binds to the polypeptide.

In another aspect, the invention features an immunogenic composition comprising a polypeptide comprising an amino acid sequence selected from the group consisting of SEQ ID NOs: 1-235, the composition when injected into a mammal elicits an immunogenic response directed against a polypeptide consisting of an amino acid sequence selected from the group consisting of SEQ ID NOs: 1-235.

The invention also features a method for treating a cancer comprising administering to a patient in need of such treatment an amount of a composition comprising a polypeptide comprising an amino acid sequence selected from the group consisting of SEQ ID NOs: 1-235 in an amount sufficient to elicit an immunogenic response.

The invention also features a method for treating a cancer patient, the method comprising administering to the patient an antibody that selectively binds to a peptide consisting of an amino acid sequence selected from the group consisting of SEQ ID NOs: 1-235.

In another aspect, the invention features a method for identifying a compound that binds to a naturally processed class I or class II MHC-binding polypeptide, the method comprising exposing a test compound to a collection of at least 100 polypeptides selected from the group consisting of polypeptides having an amino acid sequence selected from the group consisting of SEQ ID NOs: 1-235, and identifying a peptide to which the test compound binds.

An “isolated” or “purified” polypeptide, protein, or peptide (these terms are used interchangeably) is a polypeptide, protein, or peptide that is separated from those components (proteins and other naturally-occurring organic molecules) that naturally accompany it. Typically, the polypeptide, protein, or peptide is substantially pure when it constitutes at least 60%, by weight, of the protein in the preparation. Preferably, the protein in the preparation consists of at least 75%, more preferably at least 90%, and most preferably at least 99%, by weight, of the polypeptide, protein, or peptide of the invention.

An “isolated” or “purified” nucleic acid refers to a nucleic acid that is separated from other nucleic acid molecules present in the natural source of the nucleic acid. With regards to genomic DNA, the term “isolated” refers to a nucleic acid molecule that is free of sequences that naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

The term “nucleic acid” includes, for example, a recombinant DNA that is incorporated into a vector such as an autonomously replicating plasmid or virus. The nucleic acids herein can comprise ribonucleotides, deoxyribonucleotides, or modified forms of either nucleotide. Isolated nucleic acid sequences can be single or double stranded and can be polynucleotides or oligonucleotides.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The present invention relates generally to peptide sequences identified by translational profiling methods. The invention also relates to polypeptides containing the peptide sequences, nucleic acids encoding polypeptides containing the peptide sequences, the use of these compositions in methods and systems for analyzing the protein composition of cells and cell populations, and methods of using the compositions in the diagnosis and treatment of disease as well as in the screening for therapeutic compounds to treat disease.

Polypeptides and Nucleic Acids

The invention features purified polypeptides comprising a peptide sequence of any of SEQ ID NOs: 1-235. Polypeptides can be purified from cells or tissue sources using a variety of protein purification techniques.

Methods of obtaining a purified preparation of a recombinant protein are well known in the art and include culturing transformed host cells under culture conditions suitable to express the protein, and purifying the resulting protein using known purification processes, such as gel filtration or ion exchange chromatography. The purification of the protein may also utilize an affinity column containing agents which will bind to the protein; one or more column steps over affinity resins such as concanavalin A-agarose, heparin-toyopearl® or Cibacrom blue 3GA Sepharose®; one or more steps involving hydrophobic interaction chromatography using such resins as phenyl ether, butyl ether, or propyl ether; and/or immunoaffinity chromatography.

Additionally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogenous isolated protein. The protein thus purified is substantially free of other mammalian proteins and is defined in accordance with the present invention as a “purified polypeptide.”

A polypeptide can also be isolated from cells or tissue sources by using an affinity molecule to separate the polypeptide from a complex mixture of proteins. For example, a polypeptide can be purified by isolating a molecule, e.g., an MHC class I or class II molecule, to which the polypeptide is bound and eluting the polypeptide from the molecule. Alternatively, a polypeptide can be isolated from cells or tissue sources by using an anti-polypeptide antibody, e.g., an antibody described herein. Polypeptides or fragments thereof can also be synthesized chemically, e.g., by solid phase methods using an automated peptide synthesizer. Polypeptides can also be isolated and fragmented in vitro by the action of chemical or enzymatic treatments.

The amino acid sequences of the peptides of SEQ ID NOs: 1-235 are presented in Table 1 (see Examples). This table indicates the “source protein symbols” from which each of the peptides is derived. Symbols are obtained from three places in the following order: (a) gene symbol(s) and alias(es) from Locus Link; (b) gene name(s) from LocusLink; or (c) Locus titles from LocusLink. The table also provides SEQ ID NOs for each of the source proteins. The sequences corresponding to the SEQ ID NOs of these source proteins were obtained from GenBank™ accession numbers. The accession numbers can be viewed by entering (under a “Protein” search) the sequence for the “source protein reference” at www.ncbi.nlm.nih.gov/PubMed/. The entire content of each of this references is herein incorporated by reference. Many of the respective GenBank™ accessions also provide a reference to a nucleic acid sequence encoding the source protein. These nucleic acid sequences are also incorporated by reference in their entirety.

In some embodiments, the polypeptide does not include more than 200 consecutive amino acids, e.g., no more than 150, 100, 90, 80, 70, 60, 50, 40, or 30 amino acids, identical to a portion of a naturally occurring protein from which a peptide of SEQ ID NOs: 1-235 is derived. In other embodiments, the polypeptide consists of a peptide of any of SEQ ID NOs: 1-235, or a variant peptide as described below. In other embodiments, the polypeptide comprises at least 8 contiguous amino acids of an amino acid sequence selected from the group consisting of SEQ ID NOs: 1-235. In another embodiment, the purified polypeptide comprises an amino acid sequence selected from the group consisting of SEQ ID NOs: 1-235, wherein the purified polypeptide comprises at least 25 amino acids. In other embodiments, the purified polypeptide comprises fewer than 100 or 50 amino acids.

In another embodiment, the purified polypeptide consists of an amino acid sequence selected from the group consisting of SEQ ID NOs: 1-235. In another embodiment, the purified polypeptide consists essentially of an amino acid sequence selected from the group consisting of SEQ ID NOs:1-235.

Other embodiments include a polypeptide that contains one or more changes in amino acid sequence, e.g., a change in an amino acid residue that is not essential for activity, e.g., the ability of the polypeptide to bind to a MHC molecule or to be recognized by an antibody described herein. Such polypeptides differ in amino acid sequence from SEQ ID NOs: 1-235, yet retain biological activity. In one embodiment, the polypeptide includes an amino acid sequence at least about 80%, 85%, 90%, 95%, 98% or more identical to any of SEQ ID NOs: 1-235. In another embodiment the polypeptide comprises an amino acid sequence selected from the group consisting of a variant of any one of SEQ ID NOs: 1-235, wherein the variant has no more than two conservative amino acid substitutions. In another embodiment, the polypeptide comprises at least an immunogenic portion of a protein, wherein the protein comprises an amino acid sequence selected from the group consisting of SEQ ID NOs: 1-235.

The amino acid residues at particular positions in a polypeptide may include analogs, derivatives and congeners of any specific amino acid referred to herein. For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups. For instance, the subject polypeptide can include an amino acid analog such as P-cyanoalanine, canavanine, djenkolic acid, norleucine, 3-phosphoserine, homoserine, dihydroxyphenylalanine, 5-hydroxytryptophan, 1-methylhistidine, or 3-methylhistidine. Other naturally occurring amino acid metabolites or precursors having side chains that are suitable herein will be recognized by those skilled in the art and are included in the scope of the present invention. Analogs of polypeptides can be generated by mutagenesis, such as by discrete point mutation(s), or by truncation. For instance, mutation can give rise to analogs that retain substantially the same, or merely a subset, of the biological activity of the polypeptide from which it was derived.

The polypeptides that can be utilized in the present invention also include analogs that are resistant to proteolytic cleavage such as those that, due to mutations, alter ubiquitination or other enzymatic targeting associated with the protein.

Polypeptide analogs may also be chemically modified to create derivatives by forming covalent or aggregate conjugates with other chemical moieties, such as glycosyl groups, lipids, phosphate, acetyl groups and the like. Covalent derivatives of proteins can be prepared by linking the chemical moieties to functional groups on amino acid sidechains of the protein or at the N-terminus or at the C-terminus of the polypeptide.

Modification of the structure of the subject polypeptides can be for such purposes as enhancing stability (e.g., ex vivo shelf life and resistance to proteolytic degradation in vivo), or post-translational modifications (e.g., to alter the phosphorylation pattern of the polypeptide). Such modified peptides, when designed to retain at least one activity of a naturally-occurring form of the polypeptides disclosed herein, are considered to be their functional equivalents. Such modified peptides can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoeltetric mutations) will not have a major effect on the biological activity of the resulting molecule. Thus, altered nucleic acid sequences encoding polypeptides which are encompassed by the invention include deletions, insertions, or substitutions of different nucleotides resulting in a polynucleotide that encodes the same or a functionally equivalent polypeptide. The encoded protein may also contain deletions, insertions, or substitutions of amino acid residues which produce a silent change and result in a functionally equivalent polypeptide. Deliberate amino acid substitutions may be made on the basis of similarity in polarity, charge, solubility, hydrophobicity, hydrophilicity, and/or the amphipathic nature of the residues as long as the biological activity of the polypeptide is retained. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar-alanine, valine, leucine, isoleucine, proline, phenylalanine, methionine, tryptophan; and (4) uncharged polar-glycine, asparagine, glutamine, cysteine, serine, threonine, tyrosine (see, e.g., Biochemistry, 2nd ed., Ed. by L. Stryer, W H Freeman and Co.: 1981). Whether a change in the amino acid sequence of a peptide results in a functional analog (e.g., functional in the sense that the resulting polypeptide mimics the wild-type form) can be readily determined by assessing the ability of the variant peptide to produce a response in cells in a fashion similar to the wild-type protein. Polypeptides in which more than one replacement has taken place can readily be tested in the same manner.

As set forth above, alterations in primary sequence include genetic variations, both natural and induced. Also included are analogs that include residues other than naturally occurring L-amino acids, e.g., D-amino acids or non-naturally occurring or synthetic amino acids, e.g., β or δ amino acids. Alternatively, increased stability or solubility may be conferred by cyclizing the peptide molecule.

A polypeptide of the invention preferably does not contain a peptide sequence described in Tables 1-10 of U.S. Pat. No. 5,827,516.

The invention also features purified nucleic acids comprising nucleotides encoding polypeptides comprising amino acid sequences selected from the group consisting of SEQ ID NOs: 1-235 or an amino acid sequence which is at least 95% identical to an amino acid sequence selected from the group consisting of SEQ ID NOs: 1-235. In another embodiment, the isolated nucleic acid comprises a nucleotide sequence that encodes a polypeptide comprising an amino acid sequence selected from the group consisting of a variant of any one of SEQ ID NOs: 1-235, wherein the variant has no more than two conservative amino acid substitutions.

In another embodiment, the isolated nucleic acid comprises a nucleotide sequence encoding a polypeptide consisting of an amino acid sequence selected from the group consisting of SEQ ID NOs:1-235. In another embodiment, the isolated nucleic acid comprises a nucleotide sequence that encodes a polypeptide comprising at least 8 contiguous amino acids of an amino acid sequence selected from the group consisting of SEQ ID NOs:1-235. In another embodiment, the isolated nucleic acid comprises a nucleotide sequence encoding a polypeptide consisting essentially of an amino acid sequence selected from the group consisting of SEQ ID NOs:1-235.

In a further embodiment, the isolated nucleic acid comprises a nucleotide sequence encoding a polypeptide comprising no more than 30 contiguous amino acids of a naturally occurring human protein, wherein the naturally occurring protein comprises an amino acid sequence selected from the group consisting of SEQ ID NOs: 1-235.

A nucleic acid encoding a polypeptide described herein can be cloned into an expression vector, e.g., a vector in which the coding sequence is operably linked to expression control sequences. The need for, and identity of, expression control sequences will vary according to the type of cell in which the DNA is to be expressed. Generally, expression control sequences can include any or all of the following: a transcriptional promoter, enhancer, suitable mRNA ribosomal binding sites, translation start site, and sequences that terminate transcription and translation, including polyadenylation and possibly translational control sequences. Suitable expression control sequences can be selected by one of ordinary skill in the art. In one example, the vector comprises an expression control sequence that directs the expression of the polypeptides described herein. In another example, the vector comprises expression control sequences that direct expression of the nucleic acid molecule, as described herein. The nucleic acids encoding the polypeptides described herein may encode a methionine residue at the amino terminus of the polypeptide to facilitate translation. Standard methods can be used by the skilled person to construct expression vectors. See generally, Sambrook et al., 1989, Molecular Cloning: A Laboratory Manual (2nd Edition), Cold Spring Harbor Press, N.Y.

Vectors useful in this invention include linear DNA with transcriptional control elements, RNA, plasmid vectors, viral vectors, and bacterial vectors. A “plasmid” is an autonomous, self-replicating, extrachromosomal, circular DNA. Preferred viral vectors are those derived from retroviruses, adenovirus, adeno-associated virus, pox viruses, SV40 virus, alpha viruses or herpes viruses.

Isolated nucleic acids can be used for the in vitro production of polypeptides of the invention. For example, a cell or cell line can be transfected, transformed, or infected with a nucleic acid described herein. After an incubation period that permits expression of a polypeptide encoded by the nucleic acid, the polypeptide can be purified from the cell culture media, if secreted, or from a lysate of the cells expressing the polypeptide.

Fusion Proteins

The invention also provides fusion proteins. A “fusion protein” refers to a polypeptide containing a peptide sequence described herein, e.g., a peptide of any of SEQ ID NOs:1-235, and a heterologous amino acid sequence. A “heterologous amino acid sequence” refers to a sequence of contiguous amino acids that is not contained within the protein from which the peptide sequence is derived, e.g., a naturally occurring protein that contains any of SEQ D NOs: 1-235. In other words, a fusion protein is not identical to a naturally occurring protein because it contains both a peptide sequence described herein as well as an amino acid sequence not contained within the naturally occurring protein from which the peptide sequence is derived. The fusion protein can contain a heterologous amino acid sequence fused to the N-terminus and/or C-terminus of the peptide sequence.

The fusion protein can include a moiety that has a high affinity for a ligand. Such fusion proteins, e.g., GST-fusion proteins, can facilitate the purification of recombinant polypeptide. Fusion proteins can include all or a part of a serum protein, e.g., an IgG constant region, or human serum albumin.

The fusion protein can include a trafficking sequence. A “trafficking sequence” is an amino acid sequence that causes a polypeptide to which it is fused to be transported to a specific compartment of the cell. An example of a trafficking sequence is a signal sequence. In certain host cells (e.g., mammalian host cells), expression and/or secretion of a polypeptide can be increased through use of a heterologous signal sequence. For example a signal sequence can be linked, with or with out a linker, to a polypeptide described herein, e.g., a peptide of any of SEQ ID NOs:1-235.

Fusion proteins of the invention can be used as immunogens. For example, administration of a fusion protein, or a nucleic acid encoding a fusion protein, can be used to elicit an immune response in a host, e.g., a mammal such as a mouse, rat, or human. Thus, the invention features an immunogenic composition comprising a polypeptide as described herein, the composition when injected into a mammal elicits an immunogenic response directed against a polypeptide as described herein. The immunogenic response can be elicited by fragments of the polypeptide or nucleic acids encoding fragments of the polypeptide. Such fusion proteins may be useful in the development of antibodies, as described below.

Antibodies

The invention also includes an antibody, multispecific antibodies (e.g., bispecific antibodies), or a fragment thereof (e.g., an antigen-binding fragment thereof) that is specific for a peptide sequence described herein, e.g., a peptide of any of SEQ ID NOs:1-235. The term “antibody” as used herein refers to an immunoglobulin molecule or immunologically active portion thereof, i.e., an antigen-binding portion, including heterologous and chimeric antibodies. The antibody can be a polyclonal or a monoclonal antibody. In other embodiments, the antibody can be recombinantly produced, e.g., produced by phage display or by combinatorial methods. “Antibody fragments” comprise a portion of a full length antibody, generally the antigen binding or variable region thereof. Examples of antibody fragments include Fab, Fab′, F(ab′2, and Fv fragments and fragments produced by a Fab expression library; diabodies; linear antibodies; single-chain antibody molecules; and multispecific antibodies formed from antibody fragments. Neutralizing antibodies, (i.e., those which inhibit dimer formation) are especially preferred for therapeutic use. Various techniques have been developed for the production of antibody fragments. Traditionally, these fragments were derived via proteolytic digestion of intact antibodies (see, e.g., Morimoto et al., Journal of Biochemical and Biophysical Methods 24: 107-117 (1992) and Brennan et al., Science 229: 81 (1985)). However, these fragments can now be produced directly by recombinant host cells. For example, Fab′-SH fragments can be directly recovered from E. coli and chemically coupled to form F(ab′)₂ fragments (Carter et al., Bio/Technology 10: 163-167 (1992)). According to another approach, F(ab′)₂ fragments can be isolated directly from recombinant host cell culture. F(ab′)₂ fragments can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse, W. D. et al. (1989) Science 254:1275-1281). Other techniques for the production of antibody fragments will be apparent to the skilled practitioner.

It may be desirable to generate multispecific (e.g. bispecific) humanized antibodies, as described herein, having binding specificities for at least two different epitopes. Exemplary bispecific antibodies may bind to two different epitopes of a protein. Alternatively, an arm may be combined with an arm which binds to a triggering molecule on a leukocyte such as a T-cell receptor molecule (e.g., CD2 or CD3), or Fc receptors for IgG (Fc.gamma.R), such as Fc.gamma.RI (CD64), FcyRII (CD32) and Fc.gamma.RIII (CD16) so as to focus cellular defense mechanisms to the protein expressing cell. Bispecific antibodies may also be used to localize cytotoxic agents to cells which express a protein. These antibodies possess a protein-binding arm and an arm which binds the cytotoxic agent (e.g., saporin, anti-interferon-.alpha., vinca alkaloid, ricin A chain, methotrexate or radioactive isotope hapten). Bispecific antibodies can be prepared as full length antibodies or antibody fragments (e.g., F(ab′)₂ bispecific antibodies).

According to another approach for making bispecific antibodies, the interface between a pair of antibody molecules can be engineered to maximize the percentage of heterodimers which are recovered from recombinant cell culture. The preferred interface comprises at least a part of the C_{H} 3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. See WO96/27011 published Sep. 6, 1996.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′-SH fragments can be recovered from E. coli, which can be chemically coupled to form bispecific antibodies. Shalaby et al., J. Exp. Med. 175: 217-225 (1992) describe the production of a fully humanized bispecific antibody F(ab)₂ molecule. Each Fab′ fragment was separately secreted from E. coli and subjected to directed chemical coupling in vitro to form the bispecific antibody. The bispecific antibody thus formed was able to bind to cells overexpressing the HER2 receptor and normal human T cells, as well as trigger the lytic activity of human cytotoxic lymphocytes against human breast tumor targets

Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90: 6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol. 152: 5368 (1994). Alternatively, the bispecific antibody may be a “linear antibody” produced as described in Zapata et al. Protein Eng. 8(10): 1057-1062 (1995).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352: 624628 (1991) and Marks et al., J. Mol. Biol. 222: 581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81: 6851-6855 (1984)).

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹ N═C═NR, where R and R¹ are different alkyl groups.

A polypeptide described herein, e.g., a peptide of any of SEQ BD NOs:1-235, can be used as an immunogen or can be used to identify antibodies made with other immunogens, e.g., cells, membrane preparations, and the like. Polypeptides can be expressed on the cell surface enabling the binding of an antibody, as described herein, that is specific to the polypeptide. Alternately, an antibody described herein may bind to a polypeptide described herein, where the polypeptide is a target of a second antibody located on the cell surface.

An antibody (e.g., a monoclonal antibody) can be used to isolate a polypeptide described herein by standard techniques, such as affinity chromatography or immunoprecipitation. Moreover, an antibody can be used to detect the polypeptide (e.g., in a cellular lysate or cell supernatant) in order to evaluate the abundance and pattern of expression of the protein.

Furthermore, an antibody can be used to target a protein in vivo for a variety of purposes including disease screening, diagnosis, and treatment. For example, an antibody can be modified to include a toxin and/or a detectable label, as described herein. Antibodies coupled to a toxic agent can be particularly useful to target and destroy diseased or infected cells.

An antibody can be coupled to a toxin, e.g., a polypeptide toxin, e.g., ricin or diphtheria toxin or active fragment thereof, or a radioactive nucleus, or imaging agent, e.g. a radioactive, enzymatic, or other, e.g., imaging agent, e.g., a NMR contrast agent. Toxins can be optionally in an inactive state and be subject to activation following their administration to a subject (e.g., activation via radio energy, irradiation with x-rays, or other penetrating rays). Labels which produce detectable radioactive emissions or fluorescence are preferred. Examples of detectable substances that can be coupled to an antibody include various enzymes, prosthetic groups, fluorescent materials, luminescent materials, bioluminescent materials, and radioactive materials.

“Humanized” forms of non-human (e.g., murine) antibodies are chimeric antibodies which contain minimal sequence derived from non-human immunoglobulin. For the most part, humanized antibodies are human immunoglobulins (recipient antibody) in which hypervariable region residues of the recipient are replaced by hypervariable region residues from a non-human species (donor antibody) such as mouse, rat, rabbit or nonhuman primate having the desired specificity, affinity, and capacity. The resulting antibody is one in which amino acids have been replaced in the non-antigen binding regions in order to more closely resemble a human antibody, while still retaining the original binding ability. In some instances, Fv framework region OR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321: 522-525 (1986); Reichmann et al., Nature 332: 323-329 (1988); and Presta, Curr. Op. Struct. Biol. 2: 593-596 (1992).

“Single-chain Fv” or “sFv” antibody fragments comprise the V_{H} and V_{.L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Pluckthun in The Pharmacology of Monoclonal Antibodies, vol. 113, Rosenburg and Moore eds. Springer-Verlag, New York, pp. 269-315 (1994).

Humanized antibodies can be produced, for example by transgenic non-human animals. Such animals are capable of producing heterologous antibodies of multiple isotypes. Heterologous antibodies are encoded by immunoglobulin heavy chain genes not normally found in the genome of that species of non-human animal. Transgenic non-human animals (e.g., mammals) can be of a variety of species including murine (rodents (e.g., mice, rats), avian (chicken, turkey, fowl), bovine (beef, cow, cattle), ovine (lamb, sheep, goats), porcine (pig, swine), and piscine (fish). Transgenic non-human animals can be produced by introducing transgenes into the germline of the non-human animal. A “transgene” means a nucleic acid sequence (encoding, e.g., a human Fc receptor), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid. Methods of producing transgenic animals and humanized antibodies are for example described in U.S. Pat. Nos. 5,569,825, 5,770,429, and 6,11,166.

Humanized antibodies can be bound to labels or be in the form of a conjugate bound to a cytotoxic agent. The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable. The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof. A “chemotherapeutic agent” is a chemical compound useful in the treatment of cancer. Examples of chemotherapeutic agents include Adriamycin, Doxorubicin, 5-Fluorouracil, Cytosine arabinoside (“Ara-C”), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins (see U.S. Pat. No. 4,675,187), Melphalan and other related nitrogen mustards.

Covalent modifications of the humanized antibody are also included within the scope of this invention. They may be made by chemical synthesis or by enzymatic or chemical cleavage of the antibody, if applicable. Other types of covalent modifications of the antibody are introduced into the molecule by reacting targeted amino acid residues of the antibody with an organic derivatizing agent that is capable of reacting with selected side chains or the N- or C-terminal residues.

Cysteinyl residues most commonly are reacted with .alpha.-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, .alpha.-bromo-.beta.-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing .alpha.-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using ¹²⁵I or ¹³¹I to prepare labeled proteins for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyltethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the .alpha.-amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO87/05330 published 11 Sep. 1987, and in Aplin and Wriston, CRC Crit. Rev. Biochem., pp. 259-306 (1981).

Removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Hakimuddin, et al. Arch. Biochem. Biophys. 259: 52 (1987) and by Edge et al. Anal. Biochem., 118: 131 (1981). Enzymatic cleavage of carbohydrate moieties on antibodies can be achieved by the use of a variety of endo- and exo-glycosidases as described by Thotakura et al. Meth. Enzymol. 138: 350 (1987).

Another type of covalent modification of the antibody comprises linking the antibody to one of a variety of nonproteinaceous polymers, e.g., polyethylene glycol, polypropylene glycol, or polyoxyalkylenes, in the manner set forth in U.S. Pat. NOs. 4,640,835; 4,496,689; 4,301,144; 4,670,417; 4,791,192 or 4,179,337.

Humanized antibodies can also be immobilized to a solid phase. By “solid phase” is meant a non-aqueous matrix to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

Diagnostic and therapeutic uses for the antibody are contemplated. In one diagnostic application, the invention provides a method for determining the presence of a protein comprising exposing a sample suspected of containing the protein to the antibody and determining binding of the antibody to the sample. For this use, the invention provides a kit comprising the antibody and instructions for using the antibody to detect the protein.

Also included in the invention is an isolated nucleic acid, as described herein, encoding a humanized antibody, described herein, as well as a vector comprising the nucleic acid and a cell comprising the vector.

Antibodies can be used to modulate the activity of a polypeptide of the invention, as described herein. The invention includes a method for modulating the activity of the polypeptide of the invention, the method comprising contacting the polypeptide with a compound that binds to the polypeptide in a concentration sufficient to modulate the activity of the polypeptide. The compound that binds to the polypeptide can be an antibody as described herein.

The invention also features a method of making an antibody, the method comprising (a) providing a polypeptide described herein to a mammal in an amount effective to induce the production of an antibody that binds to the polypeptide; (b) isolating from the mammal a cell that produces an antibody that selectively binds to a polypeptide as described herein; (c) immortalizing the cell isolated in step (b); and (d) isolating antibodies from the immortalized cell.

The invention also includes a method of selecting an antibody, the method comprising: (a) contacting a polypeptide as described herein with an in vitro library of antibodies; (b) binding an antibody to the polypeptide; and (c) selecting the antibody that binds to the polypeptide.

The invention also includes a nucleic acid that encodes an antibody described herein. Also included are vectors that include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an antibody described herein, and method of using said cells to make an antibody.

Also included in the invention are anti-peptide antibodies. An anti-peptide antibody is an antibody that binds to the amino acid sequence of a peptide described herein, e.g., a peptide of any of SEQ ID NOs: 1-235. In one example, the antibody is capable of recognizing the peptide when the peptide is bound to an MHC class I or class II molecule. The antibody can recognize either the peptide sequence or a combination of the peptide sequence and an MHC molecule. See, e.g., Apostolopoulos et al., 1998, J. Immunol. 161:767 for a description of anti-peptide antibodies.

The anti-peptide antibodies can be used to detect the expression of a protein within a cell (e.g., detection of a processed peptide on the cell surface by an anti-peptide antibody indicates that the protein, e.g., intracellular protein, is expressed within the cell). Such an anti-peptide antibody can be particularly useful for determining the protein composition of a cell when the cell is subjected to varying conditions or stimuli. Additionally, an anti-peptide antibody can be useful for detecting the presence of a disease-associated antigen within a cell. For example, a cell can be diagnosed as containing a cancer-related protein by detecting a peptide described herein presented by an MHC molecule on the surface of the cell. Antibodies raised against peptides can also be used therapeutically to treat human maladies. For example, such an antibody can be modified to contain a reagent, e.g., a toxin, that damages or destroys diseased or infected cells to which it binds.

Gene Discovery

The human genome has been reported to contain approximately 30,000-40,000 genes, a number significantly lower than previous estimates of 100,000 or more genes. Venter et al., Science 2001 291:1304; International Human Genome Sequencing Consortium Nature 2001 409:860. One possible explanation for this discrepancy is that computer algorithms used to analyze raw nucleotide sequence and identify genes may not have detected a subset of the genes in the human genome. Because the peptides described herein correspond to portions of actual proteins actually produced by a cell, the compositions and methods of the invention allow for the identification of as yet unidentified genes. For example, those peptides that do not match to any known genes may represent the protein product of a novel gene.

A peptide sequence described herein can be compared to a predicted translation of human genomic sequence (a predicted translation of each strand of genomic DNA, in three reading frames). If this analysis identifies a matching sequence, then a careful analysis of the reading frame encoding the peptide should allow for identification of the remainder of the gene encoding the peptide, including but not limited to coding sequences, 5′ and 3′ untranslated regions, alternatively spliced exons, introns, promoters, enhancers, and silencer or repressor elements.

In addition to sequence analysis, a gene and/or a cDNA encoding a protein containing a peptide described herein can be isolated by methods well known to those of skill in the art. Isolation of a gene or a cDNA is especially relevant for peptides that lack a genomic match, but can also be useful to verify the nucleotide sequence that encodes any peptide. The skilled artisan will appreciate that a number of methods are known in the art to identify and isolate genes or cDNAs using amino acid information, and will know how to identify and practice such methods. See, for example, Sambrook et al., 1989 Molecular Cloning: A Laboratory Manual 2nd ed. Cold Spring Harbor Laboratory Press; Ausubel et al., Current Protocols in Molecular Biology. Greene Publishing Associates and Wiley Interscience, N.Y. (current edition). Such methods include the preparation of degenerate probes or primers based upon the peptide amino acid sequence and using such primers for identification and/or amplification of genes and or cDNAs in appropriate libraries or other sources of genomic materials. The chromosomal location of the gene encoding the protein from which a peptide is derived may be determined, for example, by hybridizing appropriately labeled nucleic acids to chromosomes in situ.

Detection of Protein Expression

The compositions and methods described herein can be used to determine the protein composition of a cell. The detection of mRNA within a cell, for example by Northern analysis or RT-PCR, does not indicate whether the mRNA is translated, much less how much of the corresponding protein is produced in the cell. Detection of a peptide described herein indicates that the protein from which it is derived has been produced by the cell. Thus, the invention includes a method of determining the protein composition of a cell (or tissue sample) by detecting the presence of a peptide described herein to thereby determine that the cell (or tissue sample) expresses the protein from which the peptide is derived. The method can be used to determine the presence of a peptide and/or the protein from which it is derived, and optionally the quantity of a peptide and/or protein produced by a cell.

In addition to the “translational verification” described above, the peptides can be used to determine the reading frame that is being used by a gene. For example, the detection of an mRNA or a portion of an mRNA does not automatically indicate the amino acid sequence of the corresponding protein. The peptides described herein can thus be used to discover reading frames of genes that are being expressed.

Protein Classifications

The peptides described herein belong to a wide variety of functional biological classes. Many of the classes to which particular peptides belong are described in the Table presented in the Examples. Members of many of these classes of proteins have been well-characterized as participating in important biological pathways and/or have been implicated in a variety of disease conditions. Several of these classes are described in more detail below.

Kinases

As described in Examples 1 and 2 (and the accompanying table), many of the peptides described herein are derived from proteins that appear to be kinases. Kinases catalyze the transfer of high energy phosphate groups from a phosphate donor to a phosphate acceptor. Nucleotides usually serve as the phosphate donor in these reactions, with most kinases utilizing adenosine triphosphate (ATP). Reversible protein phosphorylation is a primary method for regulating protein activity in eukaryotic cells. In general, proteins are activated by phosphorylation in response to extracellular signals such as hormones, neurotransmitters, and growth and differentiation factors. The activated proteins initiate the cell's intracellular response by way of intracellular signaling pathways and second messenger molecules, such as cyclic nucleotides, calcium-calmodulin, inositol, and various mitogens, that regulate protein phosphorylation.

Kinases are involved in many aspects of a cell's function, from basic metabolic processes such as glycolysis, to cell-cycle regulation, differentiation, and communication with the extracellular environment through signal transduction cascades. Kinase targets include proteins, inositol, lipids, and nucleotides. Inappropriate phosphorylation of proteins in cells has been linked to changes in cell cycle progression and cell differentiation. Changes in cell cycle progression have been linked to induction of apoptosis or cancer. Changes in cell differentiation have been linked to diseases and disorders of the reproductive system, immune system, and skeletal muscle.

Table 1 lists several of the peptides described herein that appear, based upon structural homology, to belong to the kinase superfamily. These peptides (and their corresponding source proteins) can therefore be used to treat disorders associated with inappropriate phosphorylation of kinase targets, e.g., disorders associated with changes in cell cycle progression and/or cell differentiation, or to screen for agonists and antagonists useful for the same purpose. In addition, nucleic acids encoding the proteins as well as compounds (e.g., antibodies) that recognize the proteins can be used in a wide variety of applications described herein, including therapeutics, diagnostics, and drug screening.

The invention therefore includes the following peptides as kinases: SEQ ID NO:7, SEQ ID NO:12, SEQ ID NO:32, SEQ ID NO:35, SEQ ID NO:36, SEQ ID NO:45, SEQ ID NO:85, SEQ ID NO:90, SEQ ID NO:95, SEQ ID NO:118, SEQ ID NO: 140, SEQ ID NO:181, and SEQ ID NO:185.

Phosphatases

As described in Examples 1 and 2 (and the accompanying table), many of the peptides described herein are derived from proteins that appear to be phosphatases. Phosphatases are characterized as tyrosine-specific or serine/threonine-specific based on their preferred phospho-amino acid substrate. Some phosphatases exhibit dual specificity for both phospho-tyrosine and phospho-serine/threonine residues.

Serine/threonine phosphatases play important roles in glycogen metabolism, muscle contraction, protein synthesis, oocyte maturation, and hepatic metabolism. (Cohen, P. (1989) Annu. Rev. Biochem. 58:453-508). Tyrosine phosphatases play important roles in lymphocyte activation and cell adhesion. In addition, the genes encoding several tyrosine phosphatases have been mapped to chromosomal regions that are translocated or rearranged in various neoplastic conditions, including lymphoma, leukemia, small cell lung carcinoma, adenocarcinoma, and neuroblastoma (Charbonneau, H. and Tonks, N. K. (1992) Annu. Rev. Cell Biol. 8:463-493). Because cellular transformation is often accompanied by increased phosphorylation activity, the regulation of phosphorylation activity by phosphatases may therefore be an important strategy for controlling some types of cancer.

Table 1 lists several of the peptides described herein that appear, based upon structural homology, to belong to the phosphatase superfamily. These peptides (and their corresponding source proteins) can therefore be used to treat disorders associated with inappropriate phosphorylation and/or phosphatase activity, or to screen for agonists and antagonists useful for the same purpose. These protein phosphatases and the nucleic acids encoding them allow for the manufacture of new compositions that are useful in the diagnosis, prevention, and treatment of disorders such as immune system disorders, cell proliferative and differentiative disorders (including cancer), and neurological disorders.

The invention therefore includes the following peptides as phosphatases: SEQ ID NO:18, SEQ ID NO:24, SEQ ID NO:76, SEQ ID NO:103, SEQ ID NO:125, SEQ ID NO:199, SEQ ID NO:224, and SEQ ID NO:231

Proteases and Protease Inhibitors

As described in Examples 1 and 2 (and the accompanying table), many of the peptides described herein are derived from proteins that appear to be proteases. Proteases cleave proteins and peptides at the peptide bond that forms the backbone of the protein or peptide chain. Proteolytic processing is an essential component of cell growth, differentiation, remodeling, and homeostasis. The cleavage of peptide bonds within cells is necessary for the maturation of precursor proteins to their active forms, the removal of signal sequences from targeted proteins, the degradation of incorrectly folded proteins, and the controlled turnover of peptides within the cell.

Proteases participate in apoptosis (and disorders associated with inappropriate levels of apoptosis) as well as tissue remodeling during embryonic development, wound healing, and normal growth. Proteases are involved in the etiology or progression of disease states such as inflammation, angiogenesis, tumor dispersion and metastasis, cardiovascular disease, neurological disease, and bacterial, parasitic, and viral infections. For example, caspases and components of caspase signaling pathways regulate apoptosis and/or inflammation in an individual.

Protease inhibitors and other regulators of protease activity control the activity and effects of proteases. Protease inhibitors have been shown to control pathogenesis in animal models of proteolytic disorders and in the treatment of HIV (Murphy, G. (1991) Agents Actions Suppl. 35:69-76).

Table 1 lists several of the peptides described herein that appear, based upon structural homology, to be proteases. These peptides (and their corresponding source proteins) can therefore be used to treat disorders associated with inappropriate protease expression or activity. Examples of such disorder include immunological disorders (including autoimmune or inflammatory disorders), angiogenesis, tumor dispersion and metastasis, cardiovascular disease, neurological disease, and pathogenic infections, or to screen for agonists and antagonists useful for the same purpose. In addition, nucleic acids encoding the proteins as well as compounds (e.g., antibodies) that recognize the proteins can be used in a wide variety of applications described herein, including therapeutics, diagnostics, and drug screening.

The invention therefore includes the following peptides as proteases: SEQ ID NO:75, SEQ ID NO:93, SEQ ID NO:163, SEQ ID NO:169. and SEQ ID NO:200.

Transporters

As described in Examples 1 and 2 (and the accompanying table), many of the peptides described herein are derived from proteins that appear to be transporters. Transporter proteins are used to facilitate the translocation of certain molecules either into or out of the cell. Often, such transporters work by “pumping” ions across the cell membrane and co-transporting specific molecules (e.g., amino acids, amino acid derivatives and precursors, dicarboxylates, or inorganic molecules) across the membrane. Such mechanisms play important roles in maintaining cellular and metabolic homeostasis, neuron function, signaling, and drug resistance. As such, transporter proteins constitute compelling targets for the development of novel therapeutic agents.

The electrical potential of a cell is generated and maintained by controlling the movement of ions across the plasma membrane. The movement of ions requires ion channels, which form ion selective pores within the membrane. There are two basic types of ion channels, ion transporters and gated ion channels. Ion transporters utilize the energy obtained from ATP hydrolysis to actively transport an ion against the ion's concentration gradient. Gated ion channels allow passive flow of an ion down the ion's electrochemical gradient under restricted conditions. Together, these types of ion channels generate, maintain, and utilize an electrochemical gradient that is used in 1) electrical impulse conduction down the axon of a nerve cell, 2) transport of molecules into cells against concentration gradients, 3) initiation of muscle contraction, and 4) endocrine cell secretion.

The etiology of numerous human diseases and disorders can be attributed to defects in the transport of molecules across membranes. Defects in the trafficking of membrane-bound transporters and ion channels are associated with several disorders, e.g., cystic fibrosis, glucose-galactose malabsorption syndrome, hypercholesterolemia, von Gierke disease, and certain forms of diabetes mellitus. Single-gene defect diseases resulting in an inability to transport small molecules across membranes include, e.g., cystinuria, iminoglycinuria, Hartup disease, and Fanconi disease (vant Hoff, W. G. (1996) Exp. Nephrol. 4:253-262; Talente, G. M. et al. (1994) Ann. Intern. Med. 120:218-226; and Chillon, M. et al. (1995) New Engl. J. Med. 332:1475-1480).

Human diseases caused by mutations in ion channel genes include disorders of skeletal muscle, cardiac muscle, and the central nervous system. Mutations in the pore forming subunits of sodium and chloride channels cause myotonia, a muscle disorder in which relaxation after voluntary contraction is delayed. Sodium channel myotonias have been treated with channel blockers. Mutations in muscle sodium and calcium channels cause forms of periodic paralysis, while mutations in the sarcoplasmic calcium release channel and muscle sodium channel cause malignant hyperthermia. Cardiac arrythmia disorders such as the long QT syndromes and idiopathic ventricular fibrillation are caused by mutations in potassium and sodium channels (Cooper, E. C. and L. Y. Jan (1998) Proc. Natl. Acad. Sci. USA 96:4759-4766). All four known human idiopathic epilepsy genes code for ion channel proteins (Berkovic, S. F. and I. E. Scheffer (1999) Curr. Opin. Neurology 12:177 182). Other neurological disorders such as ataxias and hereditary deafness can also result from mutations in ion channel genes (Jen, J. (1999) Curr. Opin. Neurobiol. 9:274-280).

Ion channels have been the target for many drug therapies. In particular, neurotransmitter-gated channels have been targeted in therapies for treatment of insomnia, anxiety, depression, and schizophrenia. Voltage-gated channels have been targeted in therapies for arrhythmia, ischemia, stroke, head trauma, and neurodegenerative disease (Taylor, C. P. and L. S. Narasimhan (1997) Adv. Pharmacol. 39:47-98).

Various classes of ion channels also play an important role in the perception of pain, and thus are potential targets for new analgesics. These include the vanilloid-gated ion channels, which are activated by the vanilloid capsaicin, as well as by noxious heat. Local anesthetics such as lidocaine and mexiletine which blockade voltage-gated ion channels have been useful in the treatment of neuropathic pain.

Ion channels in the immune system have been suggested as targets for immunomodulation. T-cell activation depends upon calcium signaling, and a diverse set of T-cell specific ion channels has been characterized that affect this signaling process. Channel blocking agents can inhibit secretion of lymphokines, cell proliferation, and killing of target cells.

Table 1 lists several of the peptides described herein that appear, based upon structural homology, to be transporters. These peptides (and their corresponding source proteins) can therefore be used to treat disorders associated with inappropriate transporter expression or activity. Examples of such disorders include neurological, muscle, and immunological disorders, or to screen for agonists and antagonists useful for the same purpose. In addition, nucleic acids encoding the proteins as well as compounds (e.g., antibodies) that recognize the proteins can be used in a wide variety of applications described herein, including therapeutics, diagnostics, and drug screening. The invention therefore includes the following peptides as transporters: SEQ ID NO:1, SEQ ID NO:25, SEQ ID NO:48, SEQ ID NO:51, SEQ ID NO:52, SEQ ID NO:56, SEQ ID NO:58, SEQ ID NO:59, SEQ ID NO:61, SEQ ID NO:62, SEQ ID NO:63, SEQ ID NO:64, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:90, SEQ ID NO:94, SEQ ID NO:100, SEQ ID NO:116, SEQ ID NO:128, SEQ ID NO:130, SEQ ID NO:131, SEQ ID NO:132, SEQ ID NO:133, SEQ ID NO:141, SEQ ID NO:170, SEQ ID NO: 178, SEQ ID NO:187, SEQ ID NO:189, SEQ ID NO:203, SEQ ID NO:207, SEQ ID NO:219, and SEQ ID NO:234.

Cytoskeletal Proteins

As described in Examples 1 and 2 (and the accompanying table), many of the peptides described herein are derived from proteins that appear to be cytoskeletal proteins. The physical-biochemical processes of cell motility, organelle movement, chromosome movement, cytokinesis, and generation of cell shape are all dependent on a complex of protein fibers found in the cytoplasm. This protein complex is termed the cytoskeleton. The cytoskeleton of eukaryotic cells has three major filamentous systems. These systems are the actin filaments, intermediate filaments, and microtubules. Each of these filamentous systems is assembled from different proteins, including actin, myosin, tubulins, and intermediate filament proteins. Different cell types and tissues express specific isoforms of the proteins which comprise these filaments. In some cases distinct isoforms and mRNA splice variants are associated with cell-type specific functions (Lees-Miller, J. P. and Helfman, D. M. (1991) BioEssays 13:429437).

Cell motility is governed by the interaction between cytoskeletal and other cellular proteins. Cytoskeletal proteins that are involved in the generation of motive force within the cell are termed contractile proteins. Cytoskeletal proteins are involved in the regulation of muscle contraction. Vertebrate smooth muscle contraction is dependent upon levels of cAMP and intracellular calcium ions.

Cytoskeletal proteins are implicated in several diseases. Pathologies such as muscular dystrophy, nephrotic syndrome, and dilated cardiomyopathy have been associated with differential expression of alpha-actinin-3 (Vainzof, M. et al. (1997) Neuropediatrics 28:223-228; Smoyer, W. E. and Mundel, P. (1998) J. Mol. Med. 76:172-183; and Sussman, M. A. et al. (1998) J. Clin. Invest. 101:51-61). Alpha actinin and several microtubule associated proteins (MAPs) are present in Hirano bodies, which are observed more frequently in the elderly and in patients with neurodegenerative diseases such as Alzheimer's disease (Maciver, S. K. and Harrington, C. R. (1995) Neuroreport. 6:1985-1988). Actinin-4, an actin-bundling protein, appears to be associated with the cell motility of metastatic cancer cells. Other disease associations include premature chromosome condensation, which is frequently observed in dividing cells from tumor tissue (Murnane, J. P.(1995) Cancer Metastasis Rev. 14:17 29), and the significant roles of axonernal and assembly MAPs in viral pathogenesis (Sodeik, B. et al. (1997) J. Cell Biol. 136:1007 1021).

Table 1 lists several of the peptides described herein that appear, based upon structural homology, to be cytoskeletal proteins. These peptides (and their corresponding source proteins) can therefore be used to treat disorders associated with inappropriate cytoskeletal protein expression or activity. Examples of such disorders include cell proliferative, immunological, vesicle trafficking, reproductive, smooth muscle, developmental, and nervous disorders, or to screen for agonists and antagonists useful for the same purpose. In addition, nucleic acids encoding the proteins as well as compounds (e.g., antibodies) that recognize the proteins can be used in a wide variety of applications described herein, including therapeutics, diagnostics, and drug screening.

The invention therefore includes the following peptides as cytoskeletal proteins: SEQ ID NO:118, SEQ ID NO: 144, SEQ ID NO:177, SEQ ID NO:183, and SEQ ID NO:185.

Receptors

As described in Examples 1 and 2 (and the accompanying table), many of the peptides described herein are derived from proteins that appear to be receptors. Receptors are a broad category of proteins that specifically recognize other molecules. Many receptors are cell surface proteins that bind extracellular ligands and produce cellular responses in the areas of growth, differentiation, endocytosis, and immune response. Other receptors facilitate the selective transport of proteins out of the endoplasmic reticulum and localize enzymes to particular locations in the cell. The propagation of cellular signals and the transport and localization of proteins rely upon specific interactions between receptors and a variety of associated proteins. Examples of families of receptors include: G-protein Coupled Receptors (GPCRs); MHC molecules; hormone receptors; and TNF receptor superfamily members.

Receptor-mediated signal transduction is the process whereby cells communicate with one another and respond to extracellular signals via a series of biochemical events. Extracellular signals are transduced through a biochemical cascade that begins with the binding of a signal molecule to a cell membrane receptor. The signal is propagated to effector molecules by intracellular signal transducing proteins and culminates with the activation of an intracellular target molecule. The process of signal transduction regulates a wide variety of cell functions including cell proliferation, cell differentiation, induction of immune responses, and gene transcription.

Table 1 lists several of the peptides described herein that appear, based upon structural homology, to be receptors. These peptides (and their corresponding source proteins) can therefore be used to treat disorders associated with inappropriate receptor expression or activity. Examples of such disorders include immunological disorders (including autoimmune/inflammatory disorders) and cell proliferative disorders (including cancer), or to screen for agonists and antagonists useful for the same purpose. In addition, nucleic acids encoding the proteins as well as compounds (e.g., antibodies) that recognize the proteins can be used in a wide variety of applications described herein, including therapeutics, diagnostics, and screening.

The invention therefore includes the following peptides as receptors: SEQ ID NO:1, SEQ ID NO:5, SEQ ID NO:6, SEQ ID NO:41, SEQ ID NO:44, SEQ ID NO:45, SEQ ID NO:46, SEQ ID NO:48, SEQ ID NO:49, SEQ ID NO:51, SEQ ID NO:53, SEQ ID NO:54, SEQ ID NO:55, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:66, SEQ ID NO:67, SEQ ID NO:69, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:86, SEQ ID NO:88, SEQ ID NO:89, SEQ ID NO:90, SEQ ID NO:91, SEQ ID NO:92, SEQ ID NO:94, SEQ ID NO: 100, SEQ ID NO: 104, SEQ ID NO:112, SEQ ID NO:121, SEQ ID NO:122, SEQ ID NO:123, SEQ ID NO:128, SEQ ID NO:129, SEQ ID NO:133, SEQ ID NO:135, SEQ ID NO: 143, SEQ ID NO: 145, SEQ ID NO: 147, SEQ ID NO:150, SEQ ID NO:161, SEQ ID NO:164, SEQ ID NO:166, SEQ ID NO:168, SEQ ID NO:170, SEQ ID NO:171, SEQ ID NO: 172, SEQ ID NO:173, SEQ ID NO: 174, SEQ ID NO:175, SEQ ID NO:176, SEQ ID NO:180, SEQ ID NO:187, SEQ ID NO:188, SEQ ID NO:194, SEQ ID NO:211, SEQ ID NO:217, SEQ ID NO:218, SEQ ID NO:219, SEQ ID NO:221, and SEQ ID NO:230.

Transcription Factors

As described in Examples 1 and 2 (and the accompanying table), many of the peptides described herein are derived from proteins that appear to be transcription factors. Regulation of gene transcription is the primary process by which a cell controls the appropriate expression of the multitude of genes necessary for growth and differentiation. The selective expression of genes at appropriate times is highly specialized in cells of multicellular organisms and permits the cells to perform “housekeeping” functions and respond to changes in their environment. These changes involve extracellular signals from a variety of sources such as hormones, neurotransmitters, and growth and differentiation factors.

Gene transcription is controlled by proteins termed transcription factors. Transcription factors act by binding to a short segment of DNA located near the site of transcription initiation. Binding of a transcription factor to the target DNA activates transcription of the gene. Transcription factors contain a variety of structural motifs that, alone or in combination with one another, permit them to recognize and bind to the wide variety of target DNA sequences.

One group of transcription factors, the TFIIIA subclass of zinc-finger proteins, is characterized by an amino acid motif (a cysteine followed by two to four amino acids, a cysteine, twelve amino acids, a histidine, three to four amino acids, and a histidine) that interacts with zinc ions. The carboxyl terminus of the TFIIIA proteins has three of these “zinc finger” motifs and specifically binds to DNA fragments containing a CACCC pattern. The amino-terminal portion of the TFIIIA proteins is proline and serine-rich and can function as a transcriptional activator. TFIIIA proteins are often important for the proper differentiation of tissues in which they are expressed.

Table 1 lists several of the peptides described herein that appear, based upon structural homology, to be transcription factors. These peptides (and their corresponding source proteins) can therefore be used to treat disorders associated with inappropriate transcription factor expression or activity, or to screen for agonists and antagonists useful for the same purpose. Examples of such disorders include cancer, arthritis, and developmental disorders. In addition, nucleic acids encoding the proteins as well as compounds (e.g., antibodies) that recognize the proteins can be used in a wide variety of applications described herein, including therapeutics, diagnostics, and screening.

The invention therefore includes the following peptides as transcription factors: SEQ ID NO:2, SEQ ID NO:10, SEQ ID NO:14, SEQ ID NO:15, SEQ ID NO:22, SEQ ED NO:27, SEQ ID NO:33, SEQ ID NO:34, SEQ ID NO:40, SEQ ID NO:43, SEQ ID NO:96, SEQ ID NO:102, SEQ ID NO:117, SEQ ID NO:120, SEQ ID NO:138, SEQ ID NO:177, SEQ ID NO:183, SEQ ID NO:184, and SEQ ID NO:208.

Therapeutics

As described above, many of the peptides of SEQ ID NOs: 1-235 belong to biological classes of proteins that have been implicated in a wide variety of disease conditions. These biological classes include kinases, phosphatases, receptors, proteases, transcription factors, transporters (such as ion channels), and cytoskeletal proteins. Additional biological classifications of many of the peptides of SEQ ID NOs:1-235 are detailed in the “biological class” column of the Table. Members of these additional classifications have also been characterized as being associated with specific disorders.

In addition to disorders associated with discrete biological classes, many of the peptides of SEQ ID NOs: 1-235 were derived from transformed cells and thus may be involved in cellular proliferative and/or differentiative disorders, e.g., cancer. The Examples and associated table describe in detail the specific transformed cell lines with which the individual peptides of the application have been found to be associated. Because these peptides have been found to be translated in transformed cells, they are expected to be useful in therapeutic, diagnostic, and screening applications as described herein. For example, in one embodiment, a compound that modulates (increases or decreases) the expression or activity of a polypeptide containing any of SEQ ID NOs: 1-235 can be used to treat or prevent a cellular proliferative and/or differentiative disorder, e.g., a B cell cancer such as myelmoa, colon cancer, gastric cancer, adenocarcinoma, sarcoma, melanoma, lymphoma, or leukemia.

In one embodiment, a polypeptide containing any of SEQ ID NOs: 1-235 (or a nucleic acid encoding such a polypeptide) can be administered to a subject to treat a disorder. For example, a disorder characterized by insufficient levels of a given polypeptide, e.g., a phosphatase or an ion channel, can be treated by such a method. In one example, a secreted protein described herein, e.g., a cytokine, is administered to a subject to treat a disorder.

In one embodiment, antagonists or inhibitors of a polypeptide containing any of SEQ ID NOs:1-235 may be administered to a subject to treat or prevent a disorder. In one aspect, antibodies specific for a polypeptide containing any of SEQ ID NOs: 1-235 may be used directly as an antagonist, or indirectly as a targeting or delivery mechanism for bringing a pharmaceutical agent to cells or tissue that expresses the polypeptide.

The invention features a method for treating cancer comprising administering to a patient in need of such treatment an amount of a composition comprising a polypeptide as described herein in an amount sufficient to elicit an immunogenic response. Also, the invention features a method for treating a cancer patient, the method comprising administering to the patient an antibody that selectively binds to a peptide as described herein.

In other embodiments, therapeutic proteins, antagonists, antibodies, agonists, antisense sequences or vectors may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

Antagonists or inhibitors of the polypeptides may be produced using methods which are generally known in the art. In particular, purified polypeptides may be used to produce antibodies or to screen libraries of pharmaceutical agents to identify those which specifically bind the polypeptide. Cells expressing a nucleic acid of the invention can be screened against the same libraries to find agents that bind and/or affect the activity of the encoded polypeptide.

An additional embodiment of the invention relates to the administration of a pharmaceutical composition, in conjunction with a pharmaceutically acceptable carrier, for any of the therapeutic effects discussed above. Such pharmaceutical compositions may consist of a polypeptide containing any of SEQ ID NOs:1-235, antibodies to the polypeptide, mimetics, agonists, antagonists, or inhibitors of the polypeptide. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.). The compositions may be administered alone or in combination with at least one other agent, such as a stabilizing compound, which may be administered in any sterile, biocompatible pharmaceutical carrier, including, but not limited to, saline, buffered saline, dextrose solution, and water. The compositions may be administered to a patient alone, or in combination with other agents, drugs or hormones.

Diagnostics

In another embodiment, compounds (e.g., antibodies) that specifically bind to a polypeptide containing any of SEQ ID NOs:1-235 may be used for the diagnosis of conditions or diseases characterized by expression of the polypeptide, or in assays to monitor patients being treated with the polypeptide, agonists, antagonists or inhibitors. Antibodies useful for diagnostic purposes may be prepared in the same manner as those prepared for therapeutic purposes. Diagnostic assays for a polypeptide containing any of SEQ ID NOs:1-235 include methods that utilize the antibody and a label to detect the polypeptide in human body fluids or extracts of cells or tissues. The antibodies may be used with or without modification, and may be labeled by joining them, either covalently or non-covalently, with a reporter molecule. A wide variety of reporter molecules that are known in the art may be used, several of which are described above.

In another embodiment of the invention, a polynucleotide, e.g., a polynucleotide encoding a polypeptide containing any of SEQ ID NOs: 1-235, may be used for diagnostic purposes. The polynucleotides that may be used include oligonucleotides, antisense RNA and DNA molecules, and PNAs. The polynucleotides may be used to detect and quantitate gene expression in biopsied tissues in which expression of a polypeptide described herein may be correlated with disease. The diagnostic assay may be used to distinguish between the absence, presence, and excess expression of an mRNA encoding a polypeptide containing any of SEQ ID NOs: 1-235, and to monitor regulation of mRNA levels during therapeutic intervention.

A polynucleotide encoding a polypeptide containing any of SEQ ID NOs:1-235 may be used for the diagnosis of conditions or diseases that are associated with expression of the polypeptide. Examples of such conditions or diseases include cancers such as cancer of the testis, colon, prostate, uterus, cervix, ovary, lung, intestine, liver, breast, skin, heart, brain, stomach, pancreas, and spleen. The polynucleotide encoding the polypeptide may be used in Southern or northern analysis, dot blot, or other membrane-based technologies; in PCR technologies; or in dip stick, pin, ELISA or chip assays utilizing fluids or tissues from patient biopsies to detect altered mRNA expression. Such qualitative or quantitative methods are well known in the art.

As the peptides described herein were found to be translated in transformed cells, these peptides can thus function as markers for a transformed cell, e.g., a cancer cell. As such, detection of polypeptides containing these peptides (or nucleic acids encoding the same) are particularly useful in the diagnosis of cellular proliferative and/or differentiative disorders such as cancer.

Screening Assays

The invention provides methods for identifying modulators, i.e., candidate or test compounds or agents (e.g., proteins, peptides, peptidomimetics, peptoids, small molecules or other drugs) which bind to a polypeptide containing any of SEQ ID NOs: 1-235, have a stimulatory or inhibitory effect on, for example, expression or activity of the polypeptide, or have a stimulatory or inhibitory effect on, for example, the expression or activity of a substrate of the polypeptide. Compounds thus identified can be used to modulate the activity of target gene products in a therapeutic protocol, to elaborate the biological function of the target gene product, or to identify compounds that disrupt normal target gene interactions.

The compounds that may be screened in accordance with the invention include, but are not limited to peptides, antibodies and fragments thereof, and other organic compounds that bind to a polypeptide containing any of SEQ ID NOs:1-235 and increase or decrease an activity of the polypeptide.

Such compounds may include, but are not limited to, peptides such as soluble peptides, including but not limited to members of random peptide libraries (Lam et al., Nature 354:82 [1991]; Houghten et al., Nature 354:84 [1991]) and combinatorial chemistry-derived molecular libraries made of D- and/or L configuration amino acids; phosphopeptides (including but not limited to members of random or partially degenerate, directed phosphopeptide libraries; Songyang et al., Cell 72:767 [1993]); antibodies (including but not limited to polyclonal, monoclonal, humanized, anti-idiotypic, chimeric and single chain antibodies; FAb, F(ab′)₂ and FAb expression library fragments; and epitope-binding fragments thereof); and small organic or inorganic molecules.

Other compounds that can be screened in accordance with the invention include but are not limited to small organic molecules that are able to gain entry into an appropriate cell and affect (1) the expression of the gene encoding a polypeptide containing any of SEQ ID NOs: 1-235 or (2) the activity of the polypeptide.

As used herein “small molecules” include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Computer modeling and searching technologies permit identification of compounds, or the improvement of already identified compounds, that can modulate expression or activity of a polypeptide containing any of SEQ ID NOs: 1-235. Having identified such a compound or composition, the active sites or regions are identified. Such active sites might typically be a binding for a natural modulator of activity. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the modulator (or ligand) is found.

Although described above with reference to design and generation of compounds that could alter binding, one could also screen libraries of known compounds, including natural products or synthetic chemicals, and biologically active materials, including proteins, for compounds which bind to a polypeptide containing any of SEQ ID NOs:1-235.

In vitro systems may be designed to identify compounds capable of interacting with a polypeptide containing any of SEQ ID NOs: 1-235. Compounds identified may be useful, for example, in the treatment of conditions such cellular proliferative and differentiative disorders, e.g., cancer.

The principle of the assays used to identify compounds that bind to a polypeptide containing any of SEQ ID NOs:1-235 involves preparing a reaction mixture of the polypeptide (or a domain thereof) and the test compound under conditions and for a time sufficient to allow the two components to interact and bind, thus forming a complex which can be removed and/or detected in the reaction mixture. The polypeptide species used can vary depending upon the goal of the screening assay. In some situations it is preferable to employ a peptide corresponding to a domain of the polypeptide fused to a heterologous protein or polypeptide that affords advantages in the assay system (e.g., labeling, isolation of the resulting complex, etc.) can be utilized.

The screening assays can be conducted in a variety of ways. For example, one method to conduct such an assay involves anchoring a peptide (or polypeptide or fusion protein) or the test substance onto a solid phase and detecting peptide/test compound complexes anchored on the solid phase at the end of the reaction. In one embodiment of such a method, the peptide reactant may be anchored onto a solid surface, and the test compound, which is not anchored, may be labeled, either directly or indirectly. The invention features a peptide array comprising at least 100 peptides selected from the group consisting of peptides as described herein, each peptide linked to a solid support at a known location. Additionally, the invention features a collection of at least 10 polypeptide arrays, each array comprising at least 100 polypeptides as described herein, each peptide linked to a solid support at a known location. Peptide arrays and methods for producing such arrays are described in, e.g., U.S. Pat. No. 5,591,646.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected, e.g., using an immobilized antibody specific for a polypeptide of the invention or the test compound to anchor any complexes formed in solution, and a labeled antibody specific for the other component of the possible complex to detect anchored complexes.

Alternatively, cell-based assays can be used to identify compounds that interact with a polypeptide containing any of SEQ ID NOs: 1-235. To this end, cell lines that express the polypeptide, or cell lines that have been genetically engineered to express the polypeptide can be used. Cell based assays are particularly useful for evaluating the functional effects of a compound identified by a screen described herein. For example, once a compound is identified based upon its ability to bind to a polypeptide of the invention, the compound can then be tested for its ability to, e.g., bind to and/or induce the selective killing of transformed cells.

Use of Peptides and Nucleic Acids Encoding Peptides to Inhibit an Immune Response

The MHC-binding peptides of SEQ ID NOs: 1-235 and the nucleic acids encoding them can be used to block MHC class I and class II-mediated antigen presentation to T cells and thereby inhibit an immune response. Inhibiting an immune response can be particularly useful in conditions such as autoimmune disorders. Methods of using “blocking peptides” to prevent MHC-mediated presentation of antigens to T cells are described in U.S. Pat. No. 5,827,516. For a polypeptide, e.g., a fusion protein, containing an MHC-binding peptide sequence of any of SEQ ID NOs: 1-235, introduction of the polypeptide (or a nucleic acid encoding the polypeptide) to a cell is expected to result in the processing and presentation of the peptide sequence in the context of an MHC class I or class II molecule.

Peptides described herein may be also useful for inhibiting an immune response when complexed with an MHC molecule, e.g., an HLA molecule, and administered to a host, e.g., a human. The use of HLA/peptide complexes to induce T cell nonresponsiveness has been described for the treatment of autoimmune conditions (see, e.g., Nag et al., 1996, Cell. Immunol. 170:25; Arimilli et al., 1996, Immunol. Cell. Biol. 74:96; Prokaeva, 2000, Curr. Opin. Investig. Drugs 1:70). In addition, antibodies directed against HLA/peptide complexes may be useful in treating disease and/or blocking T cell activation.

Use of Peptides and Nucleic Acids Encoding Peptides as References for MHC Class I and Class II Binding

Some of the peptides of SEQ ID NOs: 1-235 have been characterized as binding to MHC class I or class II molecules (see Example section). These peptides, polypeptides containing them and nucleic acids encoding the same are therefore useful as references in evaluating the ability of a test peptide to bind to an MHC molecule. For example, a peptide described herein (a “reference peptide”) can be used in a competitive assay wherein a test peptide is evaluated for its ability to compete with the reference peptide for binding to an MHC molecule. The reference peptide can optionally be labeled, e.g., with a radioactive label, and displacement of bound label in the presence of a test peptide can be measured. Alternatively, the test peptide can be labeled. Competitive peptide binding assays using a reference peptide are described in, e.g., U.S. Pat. No. 6,037,135.

Delivery Systems

The purified polypeptides, or complexes containing them (such as heat shock protein or MHC complexes), or isolated nucleic acids, can be administered using standard methods, e.g., those described in Donnelly et al. (1994) J. 1 mm. Methods 176:145, and Vitiello et al. (1995) J. Clin. Invest. 95:341. Purified polypeptides and/or isolated nucleic acids of the invention can be injected into subjects in any manner known in the art, e.g., intramuscularly, intravenously, intraarterially, intradermally, intraperitoneally, intravaginally, or subcutaneously, or they can be introduced into the gastrointestinal tract or the respiratory tract, e.g., by inhalation of a solution or powder containing the polypeptides or nucleic acids. Alternatively, the purified polypeptides or isolated nucleic acids of the invention may be applied to the skin, or electroporated into the cells or tissue. Purified polypeptides or isolated nucleic acids of the invention may be electroporated with the delivery systems (e.g. microparticles, hydrogels and polymer networks) described herein.

The purified polypeptides and isolated nucleic acids encoding polypeptides can be delivered in a pharmaceutically acceptable carrier such as saline, lipids, depot systems, hydrogels, networks, liposomes, particulates, virus-like particles, microspheres, or nanospheres; as colloidal suspensions; or as powders. The nucleic acid can be naked or associated or complexed with a delivery vehicle. For a description of the use of naked DNA, see, e.g., U.S. Pat. No. 5,693,622. For a description of the use of encapsulated DNA see, e.g., U.S. Pat. No. 5,783,567. For a description of the use of hydrogel and network delivery systems for DNA delivery see, e.g., U.S. Ser. No. 60/262,219. Nucleic acids and polypeptides can be delivered using delivery vehicles known in the art, such as lipids, liposomes, ISCOMS, microspheres, microcapsules, microparticles, gold particles, virus-like particles, nanoparticles, hydrogels or networks, polymers, condensing agents, polysaccharides, polyamino acids, dendrimers, saponins, adsorption enhancing materials, or fatty acids. Viral particles can also be used, e.g., retroviruses, adenovirus, baculovirus, adeno-associated virus, pox viruses, SV40 virus, alpha virus or herpes viruses.

It is expected that a dosage of approximately 0.1 to 100 pmoles of the polypeptide, or of about 1 to 200 μg of DNA, would be administered per kg of body weight per dose. As is well known in the medical arts, dosage for any given patient depends upon many factors, including the patient's size, body surface area, age, the particular compound to be administered, sex, time and route of administration, general health, and other drugs being administered concurrently. Determination of optimal dosage is well within the abilities of a pharmacologist of ordinary skill.

Other standard delivery methods, e.g., biolistic transfer, or ex vivo treatment, can also be used. In ex vivo treatment, cells, e.g., antigen presenting cells (APCs), dendritic cells, peripheral blood mononuclear cells, or bone marrow cells, can be obtained from a patient or an appropriate donor and treated ex vivo with a composition of the invention, and then returned to the patient.

Microparticles, including those described in U.S. Pat. No. 5,783,567 and U.S. Ser. No. 60/208,830, can be used as vehicles for delivering macromolecules such as DNA, RNA, or polypeptides into cells. Microparticles may also be made, for example, according to the methods of Mathiowitz, et al. as described in WO 95/24929, herein incorporated by reference. The microparticles can contain macromolecules embedded in a polymeric matrix or enclosed in a shell of polymer. Microparticles act to maintain the integrity of the macromolecule, e.g., by maintaining the DNA in a nondegraded state. Microparticles can also be used for pulsed delivery of the macromolecule, and for delivery at a specific site or to a specific cell or target cell population.

The polymeric matrix can be a synthetic or natural biodegradable co-polymer such as poly-lactic-co-glycolic acid, starch, gelatin, or chitin. Microparticles that are less than 10 μM in diameter can be used in particular to maximize delivery of DNA molecules into a subject's phagocytotic cells. Alternatively, microparticles that are greater than 10 μM in diameter can be injected or implanted in a tissue, where they form a deposit. As the deposit breaks down, the nucleic acid or polypeptide is released gradually over time and taken up by neighboring cells.

The purified polypeptides and isolated nucleic acids of the invention can be administered by using Immune Stimulating Complexes (ISCOMS), which are negatively charged, cage-like structures of 3040 nm in size formed spontaneously on mixing cholesterol and Quil A (saponin), or saponin alone. A polypeptide (or analog) and nucleic acid of the invention can be co-administered with an ISCOM, or the polypeptide (or analog) and nucleic acid can be administered separately. The polypeptides and nucleic acids of the invention may also be electroporated into cells or tissues of a recipient. Electroporation may occur ex vivo or in vivo.

Peptide Profiles and Databases

U.S. patent application Ser. No. 09/372,380, the content of which is herein incorporated by reference, provides compositions and methods for the characterization of a cell's protein repertoire and the storage and manipulation of that information in a computer database. A characteristic profile or fingerprint of peptides or polypeptide ligands can be generated, for example, for a given cell type, for diseased vs. normal cells, and for different metabolic or developmental states of a cell. Appropriate comparisons of the profiles can be used to identify cellular targets useful in diagnostics, drug screening and development, and delivering therapeutic regimens. The EPTs described herein, the MHC-binding peptides of SEQ ID NOs:1-235, represent a population of polypeptide ligands that can be used in the methods, ligand profiles, and databases described in U.S. Ser. No. 09/372,380. In addition to EPTs, all of the peptides described herein can be used to catalogue and profile the protein composition of a cell. The following are several non-limiting examples of uses of the peptides for identifying, cataloguing and profiling the protein composition of a cell.

Peptides and proteins from which they are derived can be used to identify, catalogue and characterize most or all proteins expressed within a cell for any given cell type, metabolic or developmental stage, and disease vs. normal state, or in response to a test substance such as a given hormone, growth factor, transcription factor, cytokine, small molecule, polypeptide, nucleic acid, carbohydrate or lipid. The approach can also identify differences between transgenic vs. non-transgenic cells, or transfected vs. non-transfected cells. As such, the invention relates to the identification of “polypeptide or peptide profiles” of a cell type of interest. These profiles can be used to pre-sort cellular proteins for “proteorics” analysis, greatly reducing the screening effort and increasing the efficiency of identifying cellular proteins involved in developmental and metabolic disease processes. Appropriate comparisons of the profiles can be used to identify cellular targets useful in diagnostics, drug screening and development, and for developing therapeutic regimens. Such data will facilitate the identification of proteins that have biological significance to a particular cellular state, e.g., in metabolism, maturation, development, disease or treatment.

Peptide esterification methods such as those described in U.S. Provisional Application No. 60/284,416, filed Apr. 16, 2001, the content of which is herein incorporated by reference, can be used to determine relative protein quantities in different cells or tissues.

Peptides of the invention can be used for comparative purposes. A distinct peptide profile, e.g., an EPT profile, can be generated for each cell of interest. The profiles of different cells, tissue or organ types of interest may be compared, and polypeptides may be identified that are differentially represented, e.g., present in one type of cell/tissue/organ, but absent from another, or expressed with different abundance. Furthermore, “differential profiles” of polypeptides may be generated representing peptides that are differentially present in the two types of cells.

Peptides described herein can be used to verify or confirm the distinct profile of a cell of interest. In this use, polypeptides from cells that are essentially identical are isolated and compared. Comparison of the peptide profiles confirms that they are essentially identical, and together represent a reproducible ligand profile for the given cell type. For example, information can be obtained if the peptide profile or set of profiles that represents polypeptides derived from two or more types of MHC molecules in the given cell type are compared. For example, a subtraction profile of polypeptides is generated from comparing polypeptides isolated from two or more types of MHC molecules.

A first cell sample and a second cell sample of interest may be obtained from different types of biological tissue (e.g., comparing smooth muscle tissue to skeletal muscle tissue), different cell types (e.g., endothelial cells and epithelial cells), different organ systems (e.g., pancreas and lung), or the same organ system but cells of different status (e.g., terminally differentiated vs. embryonic, or healthy vs. diseased or predisposed to a disease). Alternatively, one can compare transfected cells which express a particular recombinant nucleic acid versus non-transfected cells or transfected cells which do not currently express the recombinant nucleic acid. One could also compare cells treated in a particular way (either in vivo or in vitro) vs. cells treated in a different way, or untreated.

For example, a treatment may involve administration of a test substance or drug candidate such as a growth factor, a hormone, a cytokine, a small molecule, a polypeptide, a nucleic acid, a carbohydrate, or a lipid. Alternatively, a treatment may involve exposing the cells to stress conditions such as trauma, hypoxia, deprivation of glucose, deprivation of an amino acid, deprivation of a nutrient, presence of a toxin, or low or high temperature. The cells are preferably vertebrate cells (e.g., from a bird or fish), and more preferably mammalian cells, e.g., from a human or from a non-human animal such as a non-human primate, a mouse, rat, guinea pig, hamster, rabbit, dog, cat, cow, horse, pig, sheep, or goat. By using a third cell sample, one could compare three different cell samples, or compare the first sample to the second and to the third. For example, the second cell sample could be a positive control and the third cell sample a negative control, or the three cell samples could represent three different treatment regimens.

In a variation on the above, one can simply compare the proteins expressed in a first cell sample to those expressed in a reference cell sample, by generating a peptide profile, e.g., an EPT profile, that is compared to an appropriate reference peptide profile. One compares first peptide profile to the reference peptide profile, in order to identify differences or similarities between the first cell sample and the reference cell sample. This and the other comparison methods described above can be used to compare, for example, cells cultured in the presence of a test compound to cells not cultured in the presence of the test compound; or cells from an animal treated with a test compound to cells (1) from the same animal before the treatment, or (2) from a second animal not treated.

Differential peptide profiles can be generated for cells of interest where one peptide profile consists of a subset of polypeptides that is differentially present in two (or more) distinct cell types, disease stages, developmental stages, metabolic stages, cell cycle stages, treatment regimens, etc., of interest. As such, the differential profiles represent a repertoire of peptides that may directly or indirectly be involved in the different cellular phenotypes or behavior. Consequently, the differential profiles provide a valuable tool for the characterization of cell-type and/or phenotype-specific protein expression, and for the identification and/or the isolation of known or novel gene products and their respective coding sequences that are potentially involved in biological processes, such as developmental processes, establishment and progression of disease, predisposition to disease, organ development, signal transduction, differentiation, neurogenesis, etc., or in response to environmental factors or treatments. For example, the polypeptides identified as differentially expressed may be further characterized by determination of their chemical structure: i.e., sequence. Thus, the present invention provides for the characterization of differential expression, e.g., the presence or absence, of gene products encoded by known genes and/or ESTs with unknown function. The present invention thus can be used as an easy and efficient way to assign to previously identified genes or gene products a putative function and/or involvement or association with a particular developmental pathway, metabolic pathway, or disease stage. With this information, new targets for the development of gene therapy approaches and drug development may rapidly be identified.

Peptide profiles for a given cell, tissue or organ of interest can be generated and stored in a database. The compilation of data can then be used for a number of applications. First, they are used as a reference point for a human patient's or animal's sample for the diagnosis of disease, progression of disease, and predisposition for disease. For example, if a disease is associated with changes in protein composition in certain cells, organ systems, cell sources, or tissue types, a suitable patient sample may be used to generate a protein profile, and compared with profiles of corresponding samples of normal (non-diseased) and/or diseased origin to assess presence or absence of, progression of, and/or predisposition to the particular disease in question. A large number of diseases may be diagnosed this way, including diseases for which particular aberrations in protein expression are known, including, but not limited to metabolic diseases that are associated with lack of certain enzymes, proliferative diseases that are associated with aberrant expression of, e.g., oncogenes or tumor suppressors, developmental diseases that are associated with aberrant gene expression, etc. Furthermore, the peptide profiles can be used for the diagnosis of diseases or other aberrations based on pre-determined differences in EPT profiles. Thus, if it is predetermined that a given disease of interest is associated with certain changes of the peptide profile of a particular type of cell, tissue, cell source, or organ system, a human patient or animal may be diagnosed based simply on its individual profile when compared to the profiles provided by a database.

Second, peptide information can be used to detect protein translation cell, cell sample, or tissue sample. Such techniques can complement the detection of mRNA and be used to detect specific protein translation (particularly in diseased tissues).

Third, the information stored in a database may be used to identify genes and their products that are involved in the manifestation of, progression of, or predisposition to any disease of interest, and with the development of symptoms of a particular disease. For example, peptide profiles of a diseased organ, tissue or cell type may be generated and compared with the corresponding profile counterpart obtained from a non-diseased sample. Differences in the profile may be identified, and individual peptides that are differentially present in the diseased vs. the non-diseased sample may be identified and isolated for further analysis. The identified differences in the peptide profiles are useful for future diagnosis of the disease or aberration.

Generating Peptide Profiles for Different Developmental. Metabolic or Disease Stages of a Given Type of Cell

Peptide profiles for cells of different developmental, metabolic or disease stages can be generated and compared to identify differences in protein or gene expression. For example, the profiles of a cancer cell and non-cancerous cell derived from the same genetically matched tissue may be generated and compared. Proteins differentially expressed in diseased and non-diseased cells can conveniently be identified, and their involvement in disease development and progression analyzed by methods well known in the art. In this way, new targets for the treatment of the disease are efficiently identified.

Alternatively, peptide profiles of cells of different developmental stages can be generated and compared. For example, profiles of embryonic cells and adult cells derived from genetically matched tissue may be generated and compared to identify genes and their products that play a role in developmental processes, and that may be useful for the development of, e.g., novel gene therapy or other therapeutic approaches for the treatment of developmental disorders.

In another example, peptide profiles of (a) cells infected with a selected pathogen, e.g., microorganism, virus, retrovirus, or prion, and (b) corresponding non-infected cells are generated and compared to identify genes and gene products that are turned on or off in response to the infection. Alternatively, instead of being infected, the first cell can be made to take up a foreign protein or immunogenic substance, etc. This approach allows one, e.g., to identify factors produced by the cells in response to infection or introduction of the foreign substance that could be useful for therapeutic purposes.

In another example, peptide profiles from cells derived from individuals having a selected genetic disorder and individuals that do not have such disorder are generated and compared. Preferably, samples from affected and non-affected family members are used for the generation of the profiles. Depending on the particular genetic disorder chosen, cell or tissue types that are known to be affected by the particular genetic disorder are studied. In many cases, profiles of various cell and/or tissue types will be generated and compared. This example allows one to identify genes and proteins associated with a genetic disorder. The information obtained may be useful for the development of gene therapy and other therapeutic approaches and for the development of targeted drugs that interfere with the expression of genes or activity or stability of gene products that are involved in the symptoms of the genetic disease. Furthermore, this example allows selection of diagnostic targets for the identification of individuals predisposed for certain types of disease or disease symptoms.

Generation of Peptide Profiles Correlated to Response of a Given Cell Type to External Factors

In one example, a peptide profile of a given cell type treated with an external factor is generated and compared to a profile of cells of the same type which have not been so treated, to identify differences in protein expression. The cells can be recombinant or native, a cell line or non-transformed cells, or isolated directly from an animal before and after treatment of the animal with the compound.

For example, peptide profiles of cells of a selected origin or nature that have been contacted with a growth factor, cytokine or hormone, and cells that have not been contacted with the substance, but otherwise treated the same way, are generated and compared. This allows identification of genes and gene products that are turned on or turned off in response to the growth factor, cytokine or hormone, which will give, e.g., valuable insight in cellular signal transduction pathways and regulation of protein expression.

Similarly, peptide profiles of cells that have been treated with or exposed to a polypeptide, small molecule, chemokine, or nucleic acid drug or drug candidate, and cells that have not been treated with or exposed to the substance, but have otherwise been treated the same way, are generated and compared. This allows one to identify the effects of the selected substance on protein expression in the cell, and is, for example, an excellent tool for the validation of particular drugs or the identification of drugs associated with expression of a selected gene or gene product.

In another example, peptide profiles of cells that have been exposed to a selected type of compound, e.g., a selected carbohydrate or group of carbohydrates, lipid or group of lipids, amino acid or group of amino acids, nucleotide or nucleoside or group of either, or vitamin or group of vitamins, and cells that have not been treated with the compound, but have otherwise been treated the same way, are generated and compared. This allows one to identify the effects of the selected compound on the gene and protein expression of the cell, and will give valuable insight into metabolic processes.

In another example, peptide profiles of cells that have been treated with a selected nucleic acid, e.g., a selected antisense oligonucleotide, a ribozyme, an expression vector, a plasmid, an RNA, or a DNA, and cells that have not been treated with the nucleic acid, but have otherwise been treated the same way, are generated and compared. This allows one to identify the effects of the antisense oligonucleotide or other nucleic acid on the protein expression in the cell, and as such allows one to evaluate the efficacy or effect of the antisense oligonucleotide or nucleic acid. Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding polypeptides. Such DNA sequences may be incorporated into a wide variety of vectors with suitable RNA polymerase promoters such as 17 or SP6. Alternatively, these cDNA constructs that synthesize antisense RNA constitutively or inducibly can be introduced into cell lines, cells, or tissues.

Finally, peptide profiles of cells that have been subject to a selected stress condition, such as low or high temperature, hypoxia, oxidative stress, free radical-induced stress, deprivation of nutrients such as glucose, amino acids, or other essential factors, or presence of a toxin, are generated and compared to a peptide profile generated in untreated controls. Differentially expressed gene products are identified in order to give valuable insight into factors involved in cellular stress responses. This example provides an extremely valuable and efficient way to determine and/or evaluate the effect of a selected compound on protein expression in the cell. The technique may furthermore be useful to verify a desired shut-down of certain enzymatic activities, e.g., by distinguishing between phosphorylated and non-phosphorylated, or glycosylated and non-glycosylated, peptides and/or proteins. It can also be used to aid in pharmacological and/or toxicological assessment of potential new drugs, and in screening for such drugs.

Generating Peptide Profiles for Different Organ Systems

Peptide profiles of cells derived from different organs or organ systems may be generated and compared to identify differences in protein or gene expression. For example, EPT profiles of cells derived from lung, liver, heart, spleen, skin, brain, kidney, thymus, intestine, and/or colon can be generated and compared. Differentially expressed genes and proteins are thus identified. This example is useful to identify proteins that are involved in an organ's particular physiological function.

In another example, peptide profiles of selected tissue or cell types, e.g., muscle, endothelium, epithelium, neuronal, fat, ovarian, testicular, blood, bone marrow, and/or mammary tissue, etc., are generated, compared, and differentially expressed proteins identified. This will give valuable insight into a protein's involvement in a tissue or cell type's physiological function.

Generating Peptide Profiles for Expression Studies in Standard Cell Lines

Peptide profiles of cells derived from differentially engineered standard cell lines can be generated and compared to identify differences in protein expression. For example, peptide profiles of standard cell lines that have been engineered to express/overexpress one or several selected recombinant genes, e.g., genes encoding a selected growth factor receptor or other signal transduction component, transcription factor, oncogene, apoptosis-inducing gene, etc., are generated and compared to peptide profiles prepared from a reference cell line of the same origin, but which does not carry and express the selected recombinant gene. Differentially expressed genes and gene products are identified. This will allow one to identify the impact of the overexpressed gene on the expression of other polypeptides in the cell.

The following examples are not to be construed as limiting the scope of the invention in any way.

### EXAMPLES

### Example 1

### Isolation and Characterization of MHC Binding Peptides (EPTs)

This example describes peptides identified by the immunoaffinity purification of class I and class II HLA molecules, followed by acid extraction and solid phase extraction of the EPT repertoire, reversed-phase HPLC separation, and mass spectrometry analysis. Methods used to derive the peptide sequences disclosed in this example are described in detail in U.S. patent application Ser. No. 09/372,380, filed Aug. 11, 1999, the content of which is herein incorporated by reference. The various HLA molecules from which peptides were extracted are detailed in Table 1.

Table 1 describes each of the peptides according to five criteria, as follows: (1) SEQ ID NO; (2) a numeric code corresponding to cell line and HLA type; (3) SEQ ID NOs of source protein reference(s); (4) source protein symbol; and (5) a function key corresponding to biological classification(s).

The SEQ ID NO for each peptide in Table 1 is Criteria 1. The other criteria follow to the right of the peptide sequence and are separated by a vertical hatch divider. Each new peptide entry begins on the next consecutive line having the next consecutive SEQ ID NO.

Criteria 2 of Table 1 identifies a peptide according to the cell type and HLA type from which it was derived. A numeric code has been assigned to each combination of cell type and HLA type. The numeric code is as follows:

| NUMBER | CELL_LINE | HLA_TYPE |
|---|---|---|
| 1 | 721.221 | A11 |
| 2 | 721.221 | A1 |
| 3 | 721.221 | A24 |
| 4 | 721.221 | A3 |
| 5 | 721.221 | DR1 |
| 6 | 721.221 | PAN-DR |
| 7 | IM9 | A2 |
| 8 | IM9 | N/A |
| 9 | JY | A2 |
| 10 | JY | B7C7 |
| 11 | JY | DR4_13 |
| 12 | JY | DR4 |
| 13 | KATO III | A2 |
| 14 | KATO III | CLASS 1 |
| 15 | KATO III | N/A |
| 16 | KATO III | PAN-DR |
| 17 | LS174T | A2 |
| 18 | LS180 | A2 |
| 19 | LS180 | CLASS 1 |
| 20 | LS180 | PAN-CLASS 2 |
| 21 | N/A | A11 |
| 22 | NORMAL PBMC | A2 |
| 23 | NORMAL PBMC | CLASS 1 |
| 24 | PRIESS | A2 |
| 25 | PRIESS | DR4 |
| 26 | PRIESS | PAN-DR |
| 27 | SW403 | A2 |
| 28 | SW480 | A2 |
| 29 | U266 | A2 |

IM-9 is an EBV-transformed B lymphoblastoid cell line derived from the peripheral blood of a patient with multiple myeloma. This cell line is described in, e.g., Fahey et al. (1971) Ann. N.Y. Acad. Sci. 190: 221-234.

U266 is a B lymphocyte cell line established from tissue obtained from a patient with myeloma. This cell line is described in, e.g., Nilssonet al. (1970) Clin. Exp. Immunol. 7:477489.

LS 180 is a human colorectal adenocarcinoma cell line. The cell line is tumorigenic in nude mice. This cell line is described in, e.g., Tom et al. (1976) In Vitro 12:180-191. LS174T is a trypsinized variant of LS180.

SW403 and SW480 are human colorectal adenocarcinoma cell lines. The cell lines are tumorigenic in nude mice. The cell lines are described in, e.g., Fogh et al. (1977) J. Natl. Cancer Inst. 59:221-226.

KATO III is a human gastric cancer cell line. The cell line is described in, e.g., Yamamoto et al. (1996) Cancer 77:1628-33.

JY is a human lymphoblastoid cell line. The cell line is described in, e.g., J. Biol. Chem. (1979) 254:8709, J. Biol. Chem. (1975) 250:4512, and Proc Natl Acad Sci USA (1979) 76:2273.

721.221 is a human lymphoblastoid cell line that has been mutagenized to eliminate the expression of HLA-A, -B, and —C alpha chains. The cell line is described in, e.g., Shimizu et al. (1988) Proc. Natl. Acad. Sci USA 5:227-231. The 721.221 cell lines described herein were transfected with a nucleic acid encoding an individual MHC molecule, e.g., HLA-A1, -A2, -A3, or -A1.

Priess is a human B-lymphoblastoid cell line. The cell line is described in, e.g., Hanania et al. (1983) EMBO J. 2:1621-1624.

The SEQ ID NOs of the source protein reference(s) for a given peptide are described as Criteria 3 of Table 1. “Source protein” refers to an amino acid sequence or predicted amino acid sequence contained in a publicly available nucleotide and/or protein database having a region identical to an EPT sequence. In some cases, a “source protein” may not actually represent a protein from which a peptide is derived, but merely a protein (or predicted protein) containing a sequence identical to that of an EPT sequence. Peptides can be referenced to multiple different source proteins. The list of all identified source proteins for any one peptide is listed in Table 1. The sequences corresponding to the SEQ ID NOs of the source proteins are in the accompanying sequence listing.

The amino acid sequence for each of the source proteins was derived from NCBI (www.ncbi.nlm.nih.gov/PubMed/). The entire content of this reference is herein incorporated by reference.

Criteria 4, “source protein symbol,” provides the symbol identifying the source protein. Proteins may have been identified by different protein symbols in which case the different protein symbols for the source protein have been listed. Symbols are obtained from three places in the following order: (a) gene symbol(s) and alias(es) from Locus Link; (b) gene name(s) from LocusLink; or (c) Locus titles from LocusLink Criteria 5, entitled “biological classification,” provides a numeric key representing functional classifications for the peptide sequences. Several of these biological classes are described in detail in the application. All known biological classifications for a particular peptide are listed in Table 1. The numeric key corresponding to the biological class is as follows:

| FUNCTION_KEY | BIOLOGICAL FUNCTION |
|---|---|
| 1 | CYTOSKELETON |
| 2 | TUMOR SUPPRESSOR |
| 3 | DNA BINDING |
| 4 | PATHOGENESIS |
| 5 | RNA BINDING |
| 6 | RIBONUCLEOPROTEIN |
| 7 | DNA-BINDING PROTEIN |
| 8 | NUCLEUS |
| 9 | TRANSCRIPTION CO-REPRESSOR |
| 10 | POL II TRANSCRIPTION |
| 11 | DNA PACKAGING |
| 12 | TRANSFERASE |
| 13 | CHROMATIN/CHROMOSOME STRUCTURE |
| 14 | TRANSCRIPTION REGULATION |
| 15 | HISTONE ACETYLTRANSFERASE |
| 16 | TRANSCRIPTION ACTIVATING FACTOR |
| 17 | ACTIVATOR |
| 18 | INHIBITOR OR REPRESSOR |
| 19 | TRANSPORTER |
| 20 | POTASSIUM TRANSPORT |
| 21 | SULFONYLUREA RECEPTOR |
| 22 | CHANNEL [PASSIVE TRANSPORTER] |
| 23 | TRANSCRIPTION FACTOR |
| 24 | PHOSPHOPYRUVATE HYDRATASE |
| 25 | REPRESSION OF TRANSCRIPTION FROM POL II PROMOTER |
| 26 | LYASE |
| 27 | LEARNING AND MEMORY |
| 28 | SYNAPTIC TRANSMISSION |
| 29 | SMALL MOLECULE TRANSPORT |
| 30 | GLUTAMATE SIGNALLING PATHWAY |
| 31 | INTEGRAL PLASMA MEMBRANE PROTEIN |
| 32 | N-METHYL-D-ASPARTATE SELECTIVE GLUTAMATE |
| | RECEPTOR |
| 33 | NEURONAL TRANSMISSION |
| 34 | RECEPTOR (SIGNALLING) |
| 35 | MITOSIS |
| 36 | ONCOGENESIS |
| 37 | BASEMENT MEMBRANE |
| 38 | NUCLEAR CHROMOSOME |
| 39 | DNA MEDIATED TRANSFORMATION |
| 40 | CHONDROITIN SULFATE PROTEOGLYCAN |
| 41 | CHROMOSOME ORGANIZATION AND BIOGENESIS |
| 42 | MITOCHONDRION |
| 43 | AA3-TYPE CYTOCHROME-C OXIDASE |
| 44 | ENERGY GENERATION |
| 45 | CYTOSOL |
| 46 | 5′-NUCLEOTIDASE |
| 47 | HYDROLASE |
| 48 | METALLOENDOPEPTIDASE |
| 49 | PROTEOLYSIS AND PEPTIDOLYSIS |
| 50 | NEUROMUSCULAR JUNCTION DEVELOPMENT |
| 51 | PROTEASE (OTHER THAN PROTEASOMAL) |
| 52 | TRANSCRIPTION CO-FACTOR |
| 53 | TRANSCRIPTION FROM POL II PROMOTER |
| 54 | RNA POLYMERASE II TRANSCRIPTION FACTOR |
| 55 | NEGATIVE CONTROL OF CELL PROLIFERATION |
| 56 | CONTROL OF CELL PROLIFERATION |
| 57 | CELL PROLIFERATION |
| 58 | DEVELOPMENTAL PROCESSES |
| 59 | DIFFERENTIATION |
| 60 | DNA REPAIR |
| 61 | DNA REPLICATION |
| 62 | CELL CYCLE CONTROL |
| 63 | DELTA-DNA POLYMERASE COFACTOR |
| 64 | PROLIFERATING CELL NUCLEAR ANTIGEN |
| 65 | DNA SYNTHESIS |
| 66 | DNA POLYMERASE OR SUBUNIT |
| 67 | CYTOPLASM |
| 68 | SOLUBLE FRACTION |
| 69 | PROTEIN BIOSYNTHESIS |
| 70 | TRYPTOPHANYL-TRNA BIOSYNTHESIS |
| 71 | LIGASE |
| 72 | PROTEIN SYNTHESIS |
| 73 | TRNA SYNTHETASE |
| 74 | RNA-BINDING PROTEIN |
| 75 | RNA PROCESSING/MODIFICATION |
| 76 | LIPID METABOLISM |
| 77 | AMINOPHOSPHOLIPID TRANSPORT |
| 78 | AMINOPHOSPHOLIPID-TRANSPORTING ATPASE |
| 79 | ACTIVE TRANSPORTER, PRIMARY |
| 80 | AGEING |
| 81 | HELICASE |
| 82 | DNA HELICASE |
| 83 | 3′-5′ EXONUCLEASE |
| 84 | ADENOSINETRIPHOSPHATASE |
| 85 | AGING |
| 86 | NUCLEASE [ENDO, EXO, RIBO, DEOXYRIBO] |
| 87 | PROTEIN PHOSPHORYLATION |
| 88 | NON-MEMBRANE SPANNING PROTEIN TYROSINE KINASE |
| 89 | SIGNAL TRANSDUCTION |
| 90 | CHROMATIN BINDING |
| 91 | EMBRYOGENESIS AND MORPHOGENESIS |
| 92 | EMBRYONIC DEVELOPMENT |
| 93 | CHROMATIN ARCHITECTURE |
| 94 | GLUTAMINE AMIDOTRANSFERASE |
| 95 | METABOLISM OF ENERGY RESERVES |
| 96 | FRUCTOSE 6-PHOSPHATE METABOLISM |
| 97 | GLUTAMINE-FRUCTOSE-6-PHOSPHATE TRANSAMINASE |
| | (ISOMERIZING) |
| 98 | ATP BINDING |
| 99 | MTTOCHONDRIAL MEMBRANE |
| 100 | ATP-BINDING CASSETTE (ABC) TRANSPORTER |
| 101 | ATP-BINDING CASSETTE |
| 102 | RECEPTOR SIGNALLING PROTEIN |
| 103 | G-PROTEIN SIGNALLING, LINKED TO CGMP NUCLEOTIDE |
| | SECOND MESSENGER |
| 104 | PROTEIN KINASE |
| 105 | PROTEIN SERINE/THREONINE KINASE |
| 106 | NEUROGENESIS |
| 107 | NEURONAL DEVELOPMENT |
| 108 | PROLINE BIOSYNTHESIS |
| 109 | N-ACETYL-GAMMA-GLUTAMYL-PHOSPHATE REDUCTASE |
| 110 | OXIDOREDUCTASE |
| 111 | AMINO-ACID METABOLISM |
| 112 | SNRNP U5E |
| 113 | MRNA SPLICING |
| 114 | MRNA PROCESSING |
| 115 | PRE-MRNA SPLICING FACTOR |
| 116 | RNA SPLICING |
| 117 | SPLICEOSOMAL SUBUNIT |
| 118 | TRANSCRIPTION CO-ACTIVATOR |
| 119 | REPRODUCTION |
| 120 | CELL-TO-CELL SIGNALLING |
| 121 | PEROXISOMAL MEMBRANE |
| 122 | INTEGRAL PEROXISOMAL MEMBRANE |
| 123 | PEROXISOME ORGANIZATION AND BIOGENESIS |
| 124 | PEROXISOMAL LONG-CHAIN FATTY ACID IMPORT |
| 125 | CNS-SPECIFIC FUNCTIONS |
| 126 | IKB KINASE |
| 127 | IMMUNE RESPONSE |
| 128 | PHOSPHORYLATION OF I-KAPPAB |
| 129 | ANTI-PATHOGEN RESPONSE |
| 130 | ACTIN BUNDLING |
| 131 | ACTIN CYTOSKELETON |
| 132 | CELL SHAPE AND CELL SIZE CONTROL |
| 133 | ACTIN CYTOSKELETON REORGANIZATION |
| 134 | CELL STRUCTURE |
| 135 | COMPLEX ASSEMBLY PROTEIN |
| 136 | G1/S-SPECIFIC CYCLIN |
| 137 | REGULATORY SUBUNIT |
| 138 | APOPTOSIS INHIBITOR |
| 139 | HISTONE DEACETYLASE |
| 140 | ZINC BINDING |
| 141 | ANTIMICROBIAL HUMORAL RESPONSE |
| 142 | SMALL MOLECULE-BINDING PROTEIN |
| 143 | PHOSPHOMANNOMUTASE |
| 144 | PROTEIN GLYCOSYLATION |
| 145 | N-LINKED GLYCOSYLATION |
| 146 | GDP-MANNOSE BIOSYNTHESIS |
| 147 | MEMBRANE FRACTION |
| 148 | BRAIN DEVELOPMENT |
| 149 | PROTEIN BINDING |
| 150 | TRANSCRIPTION FACTOR COMPLEX |
| 151 | TRANSCRIPTION REGULATION FROM POL II PROMOTER |
| 152 | GAS EXCHANGE |
| 153 | MICROSOME |
| 154 | PLASMA MEMBRANE |
| 155 | VESICLE TARGETING |
| 156 | ER TO GOLGI TRANSPORT |
| 157 | DYSTROPHIN-ASSOCIATED GLYCOPROTEIN COMPLEX |
| 158 | ELECTRON TRANSPORTER |
| 159 | QUINOLINATE SYNTHASE |
| 160 | OTHER METABOLISM |
| 161 | CELL MOTILITY |
| 162 | INVASIVE GROWTH |
| 163 | CYTOSKELETAL STRUCTURAL PROTEIN |
| 164 | PERCEPTION OF PEST/PATHOGEN/PARASITE |
| 165 | DNA RECOMBINATION |
| 166 | PROTEIN MODIFICATION |
| 167 | DOUBLE-STRAND BREAK REPAIR |
| 168 | SPLICEOSOME |
| 169 | ENDONUCLEASE |
| 170 | BASE-EXCISION REPAIR |
| 171 | ENDODEOXYRIBONUCLEASE |
| 172 | URACIL-DNA GLYCOSYLASE |
| 173 | DNA-(APURINIC OR APYRIMIDINIC SITE) LYASE |
| 174 | CENTROMERE |
| 175 | KINETOCHORE |
| 176 | ANTI-APOPTOSIS |
| 177 | CHROMOSOME SEGREGATION |
| 178 | NUCLEAR INNER MEMBRANE, INTEGRAL PROTEIN |
| 179 | CELL DEATH/APOPTOSIS |
| 180 | ENERGY PATHWAYS |
| 181 | GLYCOGEN METABOLISM |
| 182 | 1,4-ALPHA-GLUCAN BRANCHING ENZYME |
| 183 | ENERGY STORAGE |
| 184 | CELLULAR DEFENSE RESPONSE |
| 185 | CLASS I MAJOR HISTOCOMPATIBILITY COMPLEX ANTIGEN |
| 186 | CELL ADHESION |
| 187 | ADHESIN/AGGLUTININ |
| 188 | NUCLEOLUS |
| 189 | NUCLEOPLASM |
| 190 | POLY-PYRIMIDINE TRACT BINDING |
| 191 | HETEROGENEOUS NUCLEAR RIBONUCLEOPROTEIN |
| 192 | DEFENSE RESPONSE |
| 193 | LYMPHOCYTE ANTIGEN |
| 194 | VISION |
| 195 | EXTRACELLULAR MATRIX |
| 196 | PHOTORECEPTION |
| 197 | EXTRACELLULAR MATRIX COMPONENT |
| 198 | PEROXISOME |
| 199 | CHROMATIN MODELLING |
| 200 | CELL-CELL MATRIX ADHESION |
| 201 | CELL MIGRATION/MOTILITY |
| 202 | TRANSLATION ELONGATION FACTOR |
| 203 | HYDROXYMETHYLGLUTARYL-COA SYNTHASE |
| 204 | DNA METHYLATION |
| 205 | DNA (CYTOSINE-5-)-METHYLTRANSFERASE |
| 206 | STEROL CARRIER |
| 207 | STEROL TRANSPORTER |
| 208 | ESTRADIOL 17 BETA-DEHYDROGENASE |
| 209 | INTEGRAL MEMBRANE PROTEIN |
| 210 | ETHANOLAMINEPHOSPHOTRANSFERASE |
| 211 | INDUCTION OF APOPTOSIS |
| 212 | PROTEIN KINASE CASCADE |
| 213 | CHAPERONE |
| 214 | CALCIUM BINDING |
| 215 | PROTEIN SECRETION |
| 216 | ENDOPLASMIC RETICULUM |
| 217 | ENDOPLASMIC RETICULUM MEMBRANE |
| 218 | CHAPERONES |
| 219 | PROTEIN TRANSLOCATION |
| 220 | HISTONE DEACETYLASE COMPLEX |
| 221 | CELL CYCLE REGULATOR |
| 222 | DNA DAMAGE RESPONSE, ACTIVATION OF P53 |
| 223 | HEAVY METAL BINDING |
| 224 | EPIDERMAL DEVELOPMENT AND MAINTENANCE |
| 225 | HYDROGEN-TRANSPORTING ATP SYNTHASE |
| 226 | HYDROGEN-TRANSPORTING TWO-SECTOR ATPASE |
| 227 | CHROMATIN SILENCING |
| 228 | ATP DEPENDENT RNA HELICASE |
| 229 | RESPONSE TO VIRUSES |
| 230 | ANTIVIRAL RESPONSE PROTEIN |
| 231 | PATHOGENIC INVASION |
| 232 | PEPTIDE TRANSPORT |
| 233 | PEPTIDE TRANSPORTER |
| 234 | ENZYME ACTIVATOR |
| 235 | NON-SELECTIVE VESICLE TRANSPORT |
| 236 | TRANSLATION FACTOR |
| 237 | PROTEIN SYNTHESIS ELONGATION |
| 238 | ACTIN FILAMENT |
| 239 | GTPASE ACTIVATOR |
| 240 | GTPASE INHIBITOR |
| 241 | CALMODULIN BINDING |
| 242 | PERIPHERAL PLASMA MEMBRANE PROTEIN |
| 243 | GTPASE ACTIVATING PROTEIN |
| 244 | LYSOSOME |
| 245 | LYSOSOME ORGANIZATION AND BIOGENESIS |
| 246 | RNA PROCESSING |
| 247 | PEPTIDASE |
| 248 | ARSENITE TRANSPORTER |
| 249 | NUCLEOCYTOPLASMIC TRANSPORT |
| 250 | NUCLEAR-CYTOPLASMIC TRANSPORT |
| 251 | GLUCOSE CATABOLISM |
| 252 | GLYCEROL-3-PHOSPHATE DEHYDROGENASE |
| 253 | NUTRIENT ABSORPTION |
| 254 | CYCLOPHILIN |
| 255 | ISOMERASE |
| 256 | SPECIFIC RNA POLYMERASE II TRANSCRIPTION FACTOR |
| 257 | PROTEIN FOLDING |
| 258 | HETEROCHROMATIN |
| 259 | VIRULENCE |
| 260 | H3/H4 HISTONE ACETYLTRANSFERASE |
| 261 | METALLOCARBOXYPEPTIDASE |
| 262 | CASEIN KINASE II |
| 263 | JAK-STAT CASCADE |
| 264 | ACUTE-PHASE RESPONSE |
| 265 | HEMATOPOEITIN/INTERFERON-CLASS (D200-DOMAIN) |
| | CYTOKINE RECEPTOR SIGNAL TRANSDUCER |
| 266 | SIALYLTRANSFERASE |
| 267 | AMINOSUGAR METABOLISM |
| 268 | GLYCOLIPID METABOLISM |
| 269 | LIPID: PROTEIN MODIFICATION |
| 270 | DNA TOPOISOMERASE |
| 271 | DNA TOPOISOMERASE (ATP-HYDROLYZING) |
| 272 | TOPOISOMERASE |
| 273 | DNA METABOLISM |
| 274 | DNA-DIRECTED RNA POLYMERASE I |
| 275 | TRANSCRIPTION FROM POL I PROMOTER |
| 276 | RNA POLYMERASE I TRANSCRIPTION FACTOR COMPLEX |
| 277 | POL I TRANSCRIPTION |
| 278 | RNA POLYMERASE SUBUNIT |
| 279 | TYROSINE RECOMBINASE |
| 280 | 26S PROTEASOME |
| 281 | 19S PROTEASOME REGULATORY PARTICLE |
| 282 | PROTEIN DEGRADATION |
| 283 | PROTEASOME SUBUNIT |
| 284 | ASPARTIC-TYPE ENDOPEPTIDASE |
| 285 | GUANYLATE CYCLASE |
| 286 | RECEPTOR GUANYLATE CYCLASE |
| 287 | MEIOTIC RECOMBINATION |
| 288 | MITOTIC RECOMBINATION |
| 289 | RRNA PROCESSING |
| 290 | SMALL NUCLEOLAR RNA |
| 291 | OTHER DEVELOPMENT |
| 292 | MALE MEIOSIS |
| 293 | TRANSCRIPTION FACTOR TFIIE |
| 294 | TRANSCRIPTION INITIATION FROM POL II PROMOTER |
| 295 | GENERAL RNA POLYMERASE II TRANSCRIPTION FACTOR |
| 296 | ARYLESTERASE |
| 297 | TUMOR ANTIGEN |
| 298 | INFLAMMATORY RESPONSE |
| 299 | ANTIBACTERIAL HUMORAL RESPONSE |
| 300 | RESPONSE TO PATHOGENIC BACTERIA |
| 301 | ATP DEPENDENT DNA HELICASE |
| 302 | PROTEIN COMPLEX ASSEMBLY, MULTICHAPERONE |
| | PATHWAY |
| 303 | CARBOHYDRATE METABOLISM |
| 304 | ISOCITRATE DEHYDROGENASE (NAD+) |
| 305 | GTPASE |
| 306 | GTP-BINDING PROTEIN/GTPASE |
| 307 | RRNA TRANSCRIPTION |
| 308 | TRNA TRANSCRIPTION |
| 309 | TRANSCRIPTION FACTOR TFIIIC |
| 310 | TRANSCRIPTION FROM POL III PROMOTER |
| 311 | RNA POLYMERASE III TRANSCRIPTION FACTOR |
| 312 | POL III TRANSCRIPTION |
| 313 | EXTRACELLULAR SIGNALLING CASCADE |
| 314 | GOLGI APPARATUS |
| 315 | ARF GUANYL-NUCLEOTIDE EXCHANGE FACTOR |
| 316 | GUANINE NUCLEOTIDE EXCHANGE FACTOR |
| 317 | RNA ELONGATION FROM POL II PROMOTER |
| 318 | POSITIVE TRANSCRIPTION ELONGATION FACTOR |
| 319 | MICROTUBULE |
| 320 | STRUCTURAL PROTEIN |
| 321 | GLIA CELL DIFFERENTIATION |
| 322 | PHOSPHOLIPID BINDING |
| 323 | SKELETAL DEVELOPMENT |
| 324 | CARTILAGE CONDENSATION |
| 325 | BONE DEVELOPMENT AND MAINTENANCE |
| 326 | NUCLEAR PORE |
| 327 | RAN PROTEIN BINDING |
| 328 | IMPORTIN, BETA-SUBUNIT |
| 329 | NLS-BEARING SUBSTRATE-NUCLEUS IMPORT |
| 330 | NUCLEAR LOCALIZATION SEQUENCE BINDING |
| 331 | RECEPTOR (PROTEIN TRANSLOCATION) |
| 332 | PROTEIN COMPLEX ASSEMBLY |
| 333 | PROLYL-TRNA BIOSYNTHESIS |
| 334 | GLUTAMYL-TRNA BIOSYNTHESIS |
| 335 | PROTEIN ADP-RIBOSYLATION |
| 336 | CELL GROWTH AND MAINTENANCE |
| 337 | NAD(+) ADP-RIBOSYLTRANSFERASE |
| 338 | LARGE RIBOSOMAL SUBUNIT |
| 339 | STRUCTURAL PROTEIN OF RIBOSOME |
| 340 | GENERAL CELLULAR ROLE |
| 341 | RIBOSOMAL SUBUNIT |
| 342 | MEMBRANE |
| 343 | CIRCULATION |
| 344 | POSITIVE CONTROL OF CELL PROLIFERATION |
| 345 | ANGIOGENESIS |
| 346 | HEPARIN N-DEACTYLASE/N-SULFOTRANSFERASE |
| 347 | TELOMERE MAINTENANCE |
| 348 | REGULATION OF MITOTIC RECOMBINATION |
| 349 | SINGLE-STRANDED DNA SPECIFIC |
| | ENDODEOXYRIBONUCLEASE |
| 350 | MAP KINASE |
| 351 | TGFBETA RECEPTOR SIGNALLING PATHWAY |
| 352 | TRANSLATIONAL REGULATION |
| 353 | PROTEIN KINASE INHIBITOR |
| 354 | CHEMOTAXIS |
| 355 | PHOSPHODIESTERASE I |
| 356 | PHOSPHATE METABOLISM |
| 357 | NUCLEOTIDE PYROPHOSPHATASE |
| 358 | TRANSCRIPTION FACTOR BINDING |
| 359 | G-PROTEIN LINKED RECEPTOR PROTEIN SIGNALLING |
| | PATHWAY |
| 360 | OTHER PHOSPHATASE |
| 361 | GUANYLATE KINASE |
| 362 | OTHER KINASE |
| 363 | MOTOR |
| 364 | NON-MUSCLE MYOSIN |
| 365 | MOTOR PROTEIN |
| 366 | DEOXYCYTIDINE KINASE |
| 367 | PYRIMIDINE NUCLEOTIDE METABOLISM |
| 368 | CHOLINE KINASE |
| 369 | LIPID TRANSPORT |
| 370 | HEARING |
| 371 | CELL CYCLE ARREST |
| 372 | MITOTIC G1/S TRANSITION |
| 373 | INDUCTION OF APOPTOSIS BY INTRACELLULAR SIGNALS |
| 374 | PROTEIN PHOSPHATASE 1 BINDING |
| 375 | PROTEIN KINASE A ANCHORING PROTEIN |
| 376 | ANCHOR PROTEIN |
| 377 | LIPID BINDING |
| 378 | ACTIVATION OF MAPK |
| 379 | G-PROTEIN LINKED RECEPTOR |
| 380 | PHOSPHORYLASE KINASE |
| 381 | TRANSCRIPTION TERMINATION FROM POL II PROMOTER |
| 382 | CENTRAL NERVOUS SYSTEM DEVELOPMENT |
| 383 | OXIDATIVE STRESS RESPONSE |
| 384 | CELL STRESS |
| 385 | RECEPTOR |
| 386 | CELL SURFACE RECEPTOR LINKED SIGNAL TRANSDUCTION |
| 387 | LIGAND |
| 388 | APOPTOSIS |
| 389 | CELL-CELL SIGNALLING |
| 390 | HEAT SHOCK PROTEIN |
| 391 | TRANSLATIONAL REGULATION, INITIATION |
| 392 | MRNA CLEAVAGE |
| 393 | MRNA POLYADENYLATION |
| 394 | CHOLINESTERASE |
| 395 | LAMININ RECEPTOR |
| 396 | LAMININ RECEPTOR PROTEIN |
| 397 | CYTOSOLIC SMALL RIBOSOMAL (40S)-SUBUNTT |
| 398 | FATTY ACID DESATURATION |
| 399 | EGF RECEPTOR DOWN REGULATION |
| 400 | MICROTUBULE NUCLEATION |
| 401 | MICROTUBULE ASSOCIATED PROTEIN |
| 402 | INTERLEUKIN-2 RECEPTOR |
| 403 | INTERLEUKIN-4 RECEPTOR |
| 404 | INTERLEUKIN-7 RECEPTOR |
| 405 | INTEGRIN |
| 406 | COLLAGEN BINDING |
| 407 | BLOOD COAGULATION |
| 408 | CELL ADHESION RECEPTOR |
| 409 | HISTOGENESIS AND ORGANOGENESIS |
| 410 | BLOOD CLOTTING |
| 411 | TRANSMEMBRANE RECEPTOR PROTEIN TYROSINE KINASE |
| 412 | CYTOSOLIC LARGE RIBOSOMAL (60S)-SUBUNIT |
| 413 | CLASS II MAJOR HISTOCOMPATIBILITY COMPLEX ANTIGEN |
| 414 | ATP-GATED CATION CHANNEL |
| 415 | GONAD DEVELOPMENT |
| 416 | GERM CELL MIGRATION |
| 417 | CHOLESTEROL METABOLISM |
| 418 | CHOLESTEROL BIOSYNTHESIS |
| 419 | GERMLINE MAINTENANCE |
| 420 | GOLGI CIS-FACE |
| 421 | DYNAMIN GTPASE |
| 422 | CELL COMMUNICATION |
| 423 | MITOCHONDRIAL MEMBRANE ORGANIZATION AND |
| | BIOGENESIS |
| 424 | 1-PHOSPHATIDYLINOSITOL 3-KINASE |
| 425 | INOSITOL/PHOSPHATIDYLINOSITOL KINASE |
| 426 | PROTEIN-PEROXISOME TARGETING |
| 427 | PEROXISOME TARGETING SIGNAL-1 RECEPTOR |
| 428 | RAS GTPASE ACTIVATOR |
| 429 | CELL CYCLE |
| 430 | JNK CASCADE |
| 431 | INACTIVATION OF MAPK |
| 432 | MESODERM DEVELOPMENT |
| 433 | INDUCTION OF APOPTOSIS BY EXTRACELLULAR SIGNALS |
| 434 | MISMATCH REPAIR |
| 435 | DNA REPAIR ENZYME |
| 436 | DNA REPAIR PROTEIN |
| 437 | EUKARYOTIC TRANSLATION INITIATION FACTOR 2 |
| | COMPLEX |
| 438 | EXTRACELLULAR SPACE |
| 439 | BLOOD COAGULATION FACTOR IX |
| 440 | PROTEIN PHOSPHATASE TYPE 1 CATALYST |
| 441 | REGULATION OF G-PROTEIN LINKED RECEPTOR PROTEIN |
| | SIGNALLING PATHWAY |
| 442 | METHYL TRANSFERASE |
| 443 | 3′(2′),5′-BISPHOSPHATE NUCLEOTIDASE |
| 444 | NUCLEOBASE, NUCLEOSIDE, NUCLEOTIDE AND NUCLEIC |
| | ACID METABOLISM |
| 445 | CYTOCHROME-C OXIDASE |
| 446 | LEARNING |
| 447 | FEEDING BEHAVIOR |
| 448 | PROTEIN TYROSINE KINASE |
| 449 | METHIONINE-TRNA LIGASE |
| 450 | ACTIN MODULATING |
| 451 | NUCLEOTIDE-EXCISION REPAIR |
| 452 | SINGLE-STRANDED DNA BINDING |
| 453 | EXTRACELLULAR MATRIX STRUCTURAL PROTEIN |
| 454 | ADENOSINE DEAMINASE |
| 455 | ADENOSINE DEAMINASE REACTION |
| 456 | RAS PROTEIN SIGNAL TRANSDUCTION |
| 457 | RAL GUANYL-NUCLEOTIDE EXCHANGE FACTOR |
| 458 | SMALL GTPASE REGULATORY/INTERACTING PROTEIN |
| 459 | COATED VESICLE |
| 460 | SECRETORY VESICLE |
| 461 | VESICLE TRANSPORT |
| 462 | VESICLE COAT PROTEIN |
| 463 | HIGH-DENSITY LIPOPROTEIN |
| 464 | INTERCELLULAR TRANSPORT |
| 465 | DNA DAMAGE RESPONSE |
| 466 | EYE PIGMENT BIOSYNTHESIS |
| 467 | INTRACELLULAR PROTEIN TRAFFIC |
| 468 | PROTEIN DEPHOSPHORYLATION |
| 469 | PROTEIN TYROSINE PHOSPHATASE |
| 470 | RIBOSOME BIOGENESIS |
| 471 | UBIQUITIN LIGASE COMPLEX |
| 472 | UBIQUITIN CONJUGATING ENZYME |
| 473 | UBIQUITIN-DEPENDENT PROTEIN DEGRADATION |
| 474 | PROTEIN CONJUGATION FACTOR |
| 475 | CHAPERONIN ATPASE |
| 476 | NUCLEIC ACID BINDING |
| 477 | HEAT SHOCK RESPONSE |
| 478 | NADPH:QUINONE REDUCTASE |
| 479 | PHOSPHOGLYCERATE KINASE |
| 480 | FK506 BINDING |
| 481 | MITOCHONDRIAL MATRIX |
| 482 | ELECTRON TRANSFER FLAVOPROTEIN |
| 483 | PROTEIN PHOSPHATASE TYPE 1 |
| 484 | MITOTIC CHECKPOINT |
| 485 | ANAPHASE-PROMOTING COMPLEX |
| 486 | SIGNAL RECOGNITION PARTICLE |
| 487 | DIACYLGLYCEROL KINASE |
| 488 | PHOSPHOLIPASE C ACTIVATION |
| 489 | CYTOSTOLIC CALCIUM ION CONCENTRATION ELEVATION |
| 490 | PHOSPHORIBOSYLGLYCINAMIDE FORMYLTRANSFERASE |
| 491 | TRANSLATION INITIATION FACTOR |
| 492 | EUKARYOTIC TRANSLATION INITIATION FACTOR 3 |
| | COMPLEX |
| 493 | RAN GTPASE ACTIVATOR |
| 494 | SIGNAL SEQUENCE RECEPTOR |
| 495 | CO-TRANSLATIONAL MEMBRANE TARGETING |
| 496 | DEOXYRIBONUCLEOSIDE MONOPHOSPHATE BIOSYNTHESIS |
| 497 | IMPORTIN, ALPHA-SUBUNIT |
| 498 | REGULATION OF DNA RECOMBINATION |
| 499 | NUCLEAR IMPORT/EXPORT PROTEIN |
| 500 | SPINDLE |
| 501 | CENTROSOME |
| 502 | CYTOKINESIS |
| 503 | SPINDLE POLE BODY |
| 504 | POLYSOME |
| 505 | MITOTIC SPINDLE CHECKPOINT |
| 506 | CARBAMOYL-PHOSPHATE SYNTHASE (GLUTAMINE- |
| | HYDROLYZING) |
| 507 | DEUBIQUITYLATION |
| 508 | CYSTEINE-TYPE ENDOPEPTIDASE |
| 509 | UBIQUITIN-SPECIFIC PROTEASE |
| 510 | ENDOCYTOSIS |
| 511 | RAB GTPASE ACTIVATOR |
| 512 | INSULIN RECEPTOR SIGNALLING PATHWAY |
| 513 | RNA HELICASE |
| 514 | LYSINE-TRNA LIGASE |
| 515 | NUCLEOSOME ASSEMBLY |
| 516 | CHROMATIN ASSEMBLY COMPLEX |
| 517 | CALCIUM ION TRANSPORTER |
| 518 | INOSITOL-1,4,5-TRIPHOSPHATE RECEPTOR |
| 519 | DNA-DIRECTED RNA POLYMERASE II |
| 520 | ASPARTATE CATABOLISM |
| 521 | CYTOCHROME P450 |
| 522 | EYE MORPHOGENESIS |
| 523 | EXOCYTOSIS |
| 524 | SNAP RECEPTOR |
| 525 | MEMBRANE FUSION |
| 526 | NON-SELECTIVE VESICLE TARGETING |
| 527 | DOCKING PROTEIN |
| 528 | PROTEIN TARGETING |
| 529 | REGULATION OF CDK ACTIVITY |
| 530 | EUKARYOTIC TRANSLATION INITIATION FACTOR 4 |
| | COMPLEX |
| 531 | SNRNP U2E |
| 532 | SNRNP U1E |
| 533 | SMALL NUCLEAR RIBONUCLEOPROTEIN |
| 534 | PROTEIN LOCALIZATION |
| 535 | SERPIN |
| 536 | ENZYME INHIBITOR |
| 537 | N-METHYLTRANSFERASE |
| 538 | N-TERMMAL PROTEIN METHYLATION |
| 539 | IMP CYCLOHYDROLASE |
| 540 | PHOSPHORIBOSYLAMINOIMIDAZOLECARBOXAMIDE |
| | FORMYLTRANSFERASE |
| 541 | MITOTIC G2 PHASE |
| 542 | SPINDLE POLE BODY AND MICROTUBULE CYCLE (SENSU |
| | SACCHAROMYCES) |
| 543 | GMP SYNTHASE |
| 544 | PURINE BASE BIOSYNTHESIS |
| 545 | DNA DEPENDENT DNA REPLICATION |
| 546 | DNA REPLICATION FACTOR A COMPLEX |
| 547 | NUCLEOTIDE BINDING |
| 548 | DNA REPLICATION CHECKPOINT |
| 549 | DNA REPLICATION INHIBITION |
| 550 | MITOTIC START CONTROL POINT |
| 551 | TEMPERATURE RESPONSE |
| 552 | TRANSCRIPTION |
| 553 | RECEPTOR SIGNALLING PROTEIN TYROSINE KINASE |
| 554 | DAMAGED DNA BINDING |
| 555 | PYRIMIDINE-DIMER REPAIR, DNA DAMAGE EXCISION |
| 556 | VIRAL REPLICATION |
| 557 | PROTEIN C-TERMINUS BINDING |
| 558 | PROTEIN PHOSPHATASE TYPE 2A |
| 559 | PROTEIN PHOSPHATASE TYPE 2A REGULATOR |
| 560 | NUCLEAR MEMBRANE |
| 561 | MALATE METABOLISM |
| 562 | TRICARBOXYLIC ACID CYCLE |
| 563 | MALATE DEHYDROGENASE (OXALOACETATE |
| | DECARBOXYLATING) (NADP+) |
| 564 | MEIOSIS |
| 565 | SPERMATOGENESIS |
| 566 | SISTER CHROMATID COHESION |
| 567 | CYCLIN |
| 568 | CHOLESTEROL CATABOLISM |
| 569 | STEROID HORMONE RECEPTOR |
| 570 | TRIPEPTIDYL-PEPTIDASE II |
| 571 | D-ALANYL-D-ALANINE ENDOPEPTIDASE |
| 572 | PHOSPHOLIPASE C |
| 573 | PHOSPHOLIPID METABOLISM |
| 574 | TRANSCRIPTION-COUPLED REPAIR |
| 575 | ACTIVATION OF JUN KINASE |
| 576 | ADENYLATE KINASE |
| 577 | MICROTUBULE BINDING |
| 578 | MITOTIC G2/M TRANSITION |
| 579 | STRESS RESPONSE |
| 580 | CONTROL OF HEART |
| 581 | MUSCLE CONTRACTION |
| 582 | STRUCTURAL PROTEIN OF MUSCLE |
| 583 | MUSCLE ACTION |
| 584 | EPIDERMAL DIFFERENTIATION |
| 585 | BILIVERDIN REDUCTASE |
| 586 | MAP KINASE |
| 587 | VITAMIN METABOLISM |
| 588 | RETINOID-X RECEPTOR |
| 589 | RETINOIC ACID RECEPTOR |
| 590 | IRON HOMEOSTASIS |
| 591 | CENTRIOLE |
| 592 | CENTROSOME CYCLE |
| 593 | TRANSCRIPTION ELONGATION FACTOR |
| 594 | RNA DEPENDENT ADENOSINETRIPHOSPHATASE |
| 595 | ISOPRENOID BIOSYNTHESIS |
| 596 | ISOPENTENYL-DIPHOSPHATE DELTA-ISOMERASE |
| 597 | TRANSCRIPTION REGULATION FROM POL III PROMOTER |
| 598 | CYCLIN-DEPENDENT PROTEIN KINASE |
| 599 | MUSCLE MYOSIN |
| 600 | PHOSPHOLIPASE A2 |
| 601 | MITOTIC CHROMOSOME SEGREGATION |
| 602 | OLIGOSACCHARYL TRANSFERASE |
| 603 | MYOSIN ATPASE |
| 604 | GTP BINDING |
| 605 | CELL SHAPE CONTROL |
| 606 | SMALL GTPASE MEDIATED SIGNAL TRANSDUCTION |
| 607 | HYDROGEN-TRANSPORTING ATP SYNTHASE, CATALYTIC |
| | CORE |
| 608 | MUSCLE DEVELOPMENT |
| 609 | PREGNANCY |
| 610 | UBIQUITIN--PROTEIN LIGASE |
| 611 | ACYLTRANSFERASE |
| 612 | FATTY ACID METABOLISM |
| 613 | GUANYLATE CYCLASE, SOLUBLE |
| 614 | NO MEDIATED SIGNAL TRANSDUCTION |
| 615 | FATTY ACID CATABOLISM |
| 616 | PROPIONYL-COA CARBOXYLASE |
| 617 | PROTEIN ACETYLATION |
| 618 | NUCLEOSOME REMODELLING COMPLEX |
| 619 | CASPASE-2 |
| 620 | APOPTOTIC PROGRAM |
| 621 | 7-ALPHA-HYDROXYSTEROID DEHYDROGENASE |
| 622 | INOSITOL-1,4,5-TRIPHOSPHATE 5-PHOSPHATASE |
| 623 | LIGAND-DEPENDENT NUCLEAR RECEPTOR |
| 624 | ACTIN BINDING |
| 625 | POST GOLGI TRANSPORT |
| 626 | PYRIDOXAL KINASE |
| 627 | LAMIN |
| 628 | NUCLEAR LAMINA |
| 629 | SERINE PROTEASE INHIBITOR |
| 630 | STEAROYL-COA 9-DESATURASE |
| 631 | HEPATOCYTE GROWTH FACTOR RECEPTOR |
| 632 | BILE ACID BIOSYNTHESIS |
| 633 | OXYSTEROL 7-ALPHA-HYDROXYLASE |
| 634 | POSTERIOR MIDGUT DEVELOPMENT |
| 635 | EXTRACELLULAR |
| 636 | MICROTUBULE STABILIZATION |
| 637 | EGF RECEPTOR SIGNALLING PATHWAY |
| 638 | NEUROTROPHIN TRKA RECEPTOR |
| 639 | TRANSMEMBRANE RECEPTOR PROTEIN TYROSINE KINASE |
| | SIGNALLING PATHWAY |
| 640 | METALLOPEPTIDASE |
| 641 | MITOCHONDRIAL TRANSLOCATION |
| 642 | MITOCHONDRIAL INNER MEMBRANE TRANSLOCASE |
| | COMPLEX |
| 643 | FRUCTOSE METABOLISM |
| 644 | CYTOPLASMIC DYNEIN |
| 645 | ARP2/3 PROTEIN COMPLEX |
| 646 | CELL ELONGATION |
| 647 | NADH DEHYDROGENASE (UBIQUINONE) |
| 648 | GAMETOGENESIS |
| 649 | MEIOTIC CHROMOSOME |
| 650 | DNA DAMAGE CHECKPOINT |
| 651 | MITOTIC CHROMOSOME CONDENSATION |
| 652 | DNA REPLICATION AND CHROMOSOME CYCLE |
| 653 | CAMP-DEPENDENT PROTEIN KINASE REGULATOR |
| 654 | EUKARYOTIC TRANSLATION ELONGATION FACTOR 1 |
| 655 | GOLGI MEMBRANE |
| 656 | MANNOSE BINDING LECTIN |
| 657 | PHENYLALANINE--TRNA LIGASE |
| 658 | PHENYLALANYL-TRNA BIOSYNTHESIS |
| 659 | LIGAND BINDING OR CARRIER |
| 660 | ELECTRON DONOR |
| 661 | ACYL-COA OXIDASE |
| 662 | CELL AGEING |
| 663 | DNA-DIRECTED RNA POLYMERASE III |
| 664 | TRANSCRIPTION REGULATION FROM POL I PROMOTER |
| 665 | RIBOSOME |
| 666 | SIGNAL RECOGNITION PARTICLE RECEPTOR |
| 667 | LONG-CHAIN-FATTY-ACID-COA-LIGASE |
| 668 | MONOOXYGENASE |
| 669 | TRANSLATIONAL ATTENUATION |
| 670 | TROPOMYOSIN BINDING |
| 671 | ACTIN CAPPING PROTEIN |
| 672 | CHROMATIN |
| 673 | PROTEIN-NUCLEUS IMPORT |
| 674 | LAMININ-5 |
| 675 | DEFENSE/IMMUNITY PROTEIN |
| 676 | LANOSTEROL 14-ALPHA-DEMETHYLASE |
| 677 | SH3/SH2 ADAPTOR PROTEIN |
| 678 | RHO PROTEIN SIGNAL TRANSDUCTION |
| 679 | ACTIN FILAMENT SEVERING |
| 680 | ACTIN POLYMERIZATION/DEPOLYMERIZATION |
| 681 | RAB GDP-DISSOCIATION INHIBITOR |
| 682 | XENOBIOTIC METABOLISM |
| 683 | DETOXIFICATION RESPONSE |
| 684 | CYTOCHROME B5 REDUCTASE |
| 685 | NITRIC OXIDE BIOSYNTHESIS |
| 686 | NAD(P)H DEHYDROGENASE (QUINONE) |
| 687 | SYNAPTIC TRANSMISSION, CHOLINERGIC |
| 688 | LAMIN BINDING |
| 689 | LAMIN/CHROMATIN BINDING |
| 690 | AMYLOID PROTEIN |
| 691 | MRNA BINDING |
| 692 | GDP-DISSOCIATION INHIBITOR |
| 693 | METHENYLTETRAHYDROFOLATE CYCLOHYDROLASE |
| 694 | METHYLENETETRAHYDROFOLATE DEHYDROGENASE |
| 695 | SATELLITE DNA BINDING |
| 696 | LIPID PARTICLE |
| 697 | NON-MEMBRANE SPANNING PROTEIN TYROSINE |
| | PHOSPHATASE |
| 698 | SUPEROXIDE METABOLISM |
| 699 | [EIF-5A]-DEOXYHYPUSINE SYNTHASE |
| 700 | COMPLEX I (NADH TO UBIQUINONE) |
| 701 | M PHASE |
| 702 | CYTOSKELETAL PROTEIN BINDING PROTEIN |
| 703 | PHOSPHOLIPASE A1 |
| 704 | PHOSPHATIDYLSERINE METABOLISM |
| 705 | UBIQUITIN ACTIVATING ENZYME |
| 706 | SPERMATID DEVELOPMENT |
| 707 | DNA REPLICATION ORIGIN BINDING |
| 708 | DNA REPLICATION FACTOR |
| 709 | DNA REPLICATION FACTOR C COMPLEX |
| 710 | MITOTIC G1 PHASE |
| 711 | TETRACYCLINE TRANSPORTER |
| 712 | ACTIVE TRANSPORTER, SECONDARY |
| 713 | MAJOR FACILITATOR SUPERFAMILY |
| 714 | PURINE NUCLEOTIDE BIOSYNTHESIS |
| 715 | AMIDOPHOSPHORIBOSYLTRANSFERASE |
| 716 | PROTEIN-NUCLEUS IMPORT, TRANSLOCATION |
| 717 | INTEGRAL PLASMA MEMBRANE PROTEOGLYCAN |
| 718 | DNA STRAND ELONGATION |
| 719 | TRANSKETOLASE |
| 720 | ENDOSOME |
| 721 | IRON TRANSPORT |
| 722 | TRANSFERRIN RECEPTOR |
| 723 | BLOOD PRESSURE REGULATION |
| 724 | HETEROTRIMERIC G-PROTEIN GTPASE, BETA SUBUNTT |
| 725 | ACETYL CHOLINE RECEPTOR SIGNALLING, MUSCARINIC |
| | PATHWAY |
| 726 | PROTON TRANSPORT |
| 727 | VACUOLAR HYDROGEN-TRANSPORTING ATPASE |
| 728 | SODIUM/POTASSIUM-EXCHANGING ATPASE |
| 729 | SODIUM/POTASSIUM-TRANSPORTING ATPASE |
| 730 | ANION TRANSPORT |
| 731 | MITOCHONDRIAL OUTER MEMBRANE |
| 732 | VOLTAGE-DEPENDENT ANION CHANNEL PORIN |
| 733 | APOPTOGENIC CYTOCHROME C RELEASE CHANNEL |
| 734 | ADENINE TRANSPORT |
| 735 | DOUBLE-STRANDED DNA BINDING |
| 736 | CALCIUM-TRANSPORTING ATPASE |
| 737 | GLYCOPROTEIN DEGRADATION |
| 738 | HYALURONOGLUCOSAMINIDASE |
| 739 | EXTRACELLULAR MATRIX MAINTENANCE |
| 740 | SERINE CARBOXYPEPTIDASE |
| 741 | ION CHANNEL |
| 742 | ION TRANSPORTER |
| 743 | SENSORY PERCEPTION |
| 744 | PAIN SENSATION |
| 745 | THERMORECEPTION AND RESPONSE |
| 746 | CYTOSOLIC RIBOSOME |
| 747 | L-LACTATE DEHYDROGENASE |
| 748 | HETEROTRIMERIC G-PROTEIN GTPASE, GAMMA SUBUNIT |
| 749 | RAB SMALL MONOMERIC GTPASE |
| 750 | RNA POLYMERASE I TRANSCRIPTION FACTOR |
| 751 | C-5 STEROL DESATURASE |
| 752 | CATABOLISM |
| 753 | CARBOXYLESTERASE |
| 754 | ADDICTION |
| 755 | VITAMIN B12 TRANSPORT |
| 756 | PHYSIOLOGICAL PROCESSES |
| 757 | VITAMIN BIOSYNTHESIS |
| 758 | CALCIUM ION HOMEOSTASIS |
| 759 | CALCIDIOL 1-MONOOXYGENASE |
| 760 | OOGENESIS |
| 761 | CYSTEINE-TYPE PEPTIDASE |
| 762 | G-PROTEIN COUPLED RECEPTOR PROTEIN SIGNALING |
| | PATHWAY |
| 763 | PROTEIN TYROSINE/THREONINE PHOSPHATASE |
| 764 | DYNACTIN COMPLEX |
| 765 | INTEGRIN LIGAND |
| 766 | INTEGRIN RECEPTOR SIGNAL SIGNALLING PATHWAY |
| 767 | NEGATIVE REGULATION OF HOMEOTIC GENE (POLYCOMB |
| | GROUP) |
| 768 | FOCAL ADHESION |
| 769 | SPECTRIN |
| 770 | CELL DEATH |
| 771 | FERRITIN |
| 772 | IRON BINDING |
| 773 | PEPTIDYLPROLYL ISOMERASE |
| 774 | MICROTUBULE CYTOSKELETON |
| 775 | GAMMA-AMINOBUTYRIC ACID-INHIBITED CHLORIDE |
| | CHANNEL |
| 776 | GLUTATHIONE SYNTHASE |
| 777 | AMINO ACID METABOLISM |
| 778 | TRANSMEMBRANE RECEPTOR |
| 779 | EXCRETION |
| 780 | TRANSCRIPTION REGULATION, FROM POL II PROMOTER |
| 781 | CASPASE ACTIVATION |
| 782 | STAT PROTEIN DIMERIZATION |
| 783 | NIK-I-KAPPAB/NF-KAPPAB CASCADE |
| 784 | RESPONSE TO PEST/PATHOGEN/PARASITE |
| 785 | STAT PROTEIN NUCLEAR TRANSLOCATION |
| 786 | TYROSINE PHOSPHORYLATION OF STAT PROTEIN |
| 787 | METABOTROPIC GLUTAMATE RECEPTOR |
| 788 | METABOTROPHIC GLUTAMATE RECEPTOR, PHOSPHOLIPASE |
| | C ACTIVATING PATHWAY |
| 789 | HUMORAL DEFENSE MECHANISM |
| 790 | INTERCELLULAR JUNCTION |
| 791 | PEROXISOME-ASSEMBLY ATPASE |
| 792 | PHOSPHATE CARRIER |
| 793 | MITOCHONDRIAL INNER MEMBRANE |
| 794 | PROTEIN KINASE C BINDING PROTEIN |
| 795 | MITOTIC S-SPECIFIC TRANSCRIPTION |
| 796 | INDUCTION OF APOPTOSIS BY DNA DAMAGE |
| 797 | RECOMBINATION |
| 798 | AXON GUIDANCE RECEPTOR |
| 799 | COPII VESICLE |
| 800 | SNRNA TRANSCRIPTION |
| 801 | PROTEIN DEGRADATION TAGGING |
| 802 | ELECTRON TRANSPORT |
| 803 | AEROBIC RESPIRATION |
| 804 | RESPIRATORY CHAIN COMPLEX |
| 805 | CYTOCHROME C OXIDASE BIOGENESIS |
| 806 | TFIID COMPLEX |
| 807 | CYANATE CATABOLISM |
| 808 | THIOSULFATE SULFURTRANSFERASE |
| 809 | STRIATED MUSCLE CONTRACTION REGULATION |
| 810 | ENHANCER BINDING |
| 811 | MICROTUBULE CYTOSKELETON ORGANIZATION AND |
| | BIOGENESIS |
| 812 | RHO GUANYL-NUCLEOTIDE EXCHANGE FACTOR |
| 813 | LACTOSE BIOSYNTHESIS |
| 814 | PROGENY NUTRITION |
| 815 | VASCULAR ENDOTHELIAL GROWTH FACTOR RECEPTOR |
| 816 | PHOSPHOLIPASE INHIBITOR |
| 817 | APOLIPOPROTEIN |
| 818 | LOW-DENSITY LIPOPROTEIN |
| 819 | INTRACELLULAR IRON STORAGE |
| 820 | OTHER |
| 821 | SCAVENGER RECEPTOR |
| 822 | RNA CATABOLISM |
| 823 | POLY(A) BINDING |
| 824 | RNA TURNOVER |
| 825 | LDL RECEPTOR |
| 826 | LIPOPROTEIN BINDING |
| 827 | O-LINKED GLYCOSYLATION |
| 828 | LOW-DENSITY LIPOPROTEIN RECEPTOR |
| 829 | PURINE BASE METABOLISM |
| 830 | RIBOSE-PHOSPHATE PYROPHOSPHOKINASE |
| 831 | CARBOXYPEPTIDASE |
| 832 | INTRACELLULAR TRANSPORTER |
| 833 | ESTERASE, UNKNOWN SUBSTRATE |
| 834 | EARLY ENDOSOME |
| 835 | VESICLE FUSION |
| 836 | GLUCOSE TRANSPORT |
| 837 | GLUCOSE TRANSPORTER |
| 838 | INTERNALIZATION RECEPTOR |
| 839 | RECEPTOR MEDIATED ENDOCYTOSIS |
| 840 | CARBOXYPEPTIDASE D |
| 841 | DEOXYRIBONUCLEASE |
| 842 | TGFBETA RECEPTOR COMPLEX ASSEMBLY |
| 843 | INTERFERON-GAMMA RECEPTOR |
| 844 | RESISTANCE TO PATHOGENIC BACTERIA |
| 845 | SPHINGOMYELIN METABOLISM |
| 846 | SPHINGOMYELIN PHOSPHODIESTERASE |
| 847 | POST-REPLICATION REPAIR |
| 848 | DIGESTION |
| 849 | PEPTIDE HORMONE |
| 850 | NUTRITIONAL RESPONSE PATHWAY |
| 851 | INDUCTION OF APOPTOSIS BY HORMONES |
| 852 | ALPHA-MANNOSIDASE |
| 853 | PROTEIN DEGLYCOSYLATION |
| 854 | PHOSPHORIBOSYLAMINOIMIDAZOLE- |
| | SUCCINOCARBOXAMIDE SYNTHASE |
| 855 | BETA-N-ACETYLHEXOSAMINIDASE |
| 856 | ALKYL HYDROPEROXIDE REDUCTASE |
| 857 | ACTIN MONOMER BINDING |
| 858 | TRANSMEMBRANE RECEPTOR PROTEIN TYROSINE |
| | PHOSPHATASE |
| 859 | PROTEOGLYCAN |
| 860 | ENZYME |
| 861 | CERAMIDE METABOLISM |
| 862 | LATE ENDOSOME |
| 863 | HYALURONIC ACID BINDING |
| 864 | COPPER BINDING |
| 865 | COPPER HOMEOSTASIS |
| 866 | COPPER ION TRANSPORT |
| 867 | MRNA CAP BINDING |
| 868 | PROTEIN KINASE C INHIBITOR |
| 869 | CAMP-DEPENDENT PROTEIN KINASE |
| 870 | ADENINE TRANSPORTER |
| 871 | MITOCHONDRIAL GENOME MAINTENANCE |
| 872 | HEPARIN BINDING |
| 873 | PROCOLLAGEN-LYSINE 5-DIOXYGENASE |
| 874 | MALE GONAD DEVELOPMENT |
| 875 | MALE SPECIFIC DEVELOPMENT |
| 876 | NUCLEOSIDE METABOLISM |
| 877 | NUCLEOSIDE-DIPHOSPHATE KINASE |
| 878 | GLYCOSAMINOGLYCAN CATABOLISM |
| 879 | COPPER-EXPORTING ATPASE |
| 880 | GOLGI TRANS-FACE |
| 881 | GOLGI-PLASMA MEMBRANE TRANSPORT VESICLE |
| 882 | TRANSCRIPTION FACTOR TFIIH |
| 883 | HEMOCYTE DEVELOPMENT |
| 884 | TRANSMEMBRANE RECEPTOR PROTEIN TYROSINE KINASE |
| | ADAPTOR PROTEIN |
| 885 | DNA REPLICATION INITIATION |
| 886 | ALDEHYDE REDUCTASE |
| 887 | FAT BODY DEVELOPMENT |
| 888 | TRIACYLGLYCEROL METABOLISM |
| 889 | DIACYLGLYCEROL O-ACYLTRANSFERASE |
| 890 | CASEIN KINASE I |
| 891 | ROUGH ENDOPLASMIC RETICULUM |
| 892 | DOUBLE-STRANDED RNA BINDING |
| 893 | UV RESPONSE |
| 894 | TRANSCRIPTION INITIATION |
| 895 | DNA-NONHOMOLOGOUS END-JOINING |
| 896 | SODIUM TRANSPORT |
| 897 | SODIUM: PHOSPHATE SYMPORTER |
| 898 | CIRCADIAN RHYTHM |
| 899 | LOCOMOTORY BEHAVIOR |
| 900 | NEUROPEPTIDE Y RECEPTOR |
| 901 | CALCIUM CHANNEL REGULATOR |
| 902 | G-PROTEIN SIGNALLING, ADENYLATE CYCLASE INHIBITING |
| | PATHWAY |
| 903 | FEEDING |
| 904 | LOCOMOTION |
| 905 | RIBONUCLEASE P |
| 906 | PROTEIN-NUCLEUS IMPORT, DOCKING |
| 907 | EXIT FROM MITOSIS |
| 908 | SEPTIN ASSEMBLY AND SEPTUM FORMATION |
| 909 | RESPONSE TO INJURY |
| 910 | DELTA DNA POLYMERASE |
| 911 | INTERMEDIATE FILAMENT |
| 912 | CONTROL OF MITOSIS |
| 913 | SINGLE-STRANDED RNA BINDING |
| 914 | FORMATE-TETRAHYDROFOLATE LIGASE |
| 915 | CALCIUM ION TRANSPORT |
| 916 | N-ACETYLTRANSFERASE |
| 917 | INTERNAL PROTEIN ACETYLATION |
| 918 | MITOTIC SPINDLE ASSEMBLY |
| 919 | RAN SMALL MONOMERIC GTPASE |
| 920 | UBIQUITINYL HYDROLASE 1 |
| 921 | KDEL RECEPTOR |
| 922 | STEROID BIOSYNTHESIS |
| 923 | CELL-SUBSTRATE JUNCTION ASSEMBLY |
| 924 | MILK PROTEIN |
| 925 | CITRATE LYASE |
| 926 | ATP CATABOLISM |
| 927 | CITRATE METABOLISM |
| 928 | COENZYME A METABOLISM |
| 929 | ATP-CITRATE (PRO-S)-LYASE |
| 930 | PROTEIN KINASE C ACTIVATION |
| 931 | PROTEIN KINASE INHIBITION |
| 932 | INSULIN-LIKE GROWTH FACTOR RECEPTOR |
| 933 | HEME BIOSYNTHESIS |
| 934 | PORPHOBILINOGEN SYNTHASE |
| 935 | PLASMA PROTEIN |
| 936 | THYROID HORMONE TRANSPORTER |
| 937 | CONSERVED ATPASE DOMAIN |
| 938 | UBIQUITIN ISOPEPTIDASE T |
| 939 | LYSOSOMAL CYSTEINE-TYPE ENDOPEPTIDASE |
| 940 | DNA LIGASE |
| 941 | GLYCOLYSIS |
| 942 | STRIATED MUSCLE CONTRACTION |
| 943 | FRUCTOSE-BISPHOSPHATE ALDOLASE |
| 944 | VOLTAGE-GATED POTASSIUM CHANNEL |
| 945 | INWARD RECTIFIER POTASSIUM CHANNEL |
| 946 | MACROPHAGE ELASTASE |
| 947 | CONNECTIVE TISSUE DEVELOPMENT AND MAINTENANCE |
| 948 | SERINE-TYPE PEPTIDASE |
| 949 | 7S RNA BINDING |
| 950 | 3′-5′ EXORIBONUCLEASE |
| 951 | PROTEIN C (ACTIVATED) |
| 952 | IRON-SULFUR ELECTRON TRANSFER CARRIER |
| 953 | MHC-INTERACTING PROTEIN |
| 954 | CELL CYCLE CHECKPOINT |
| 955 | MYOBLAST DETERMINATION |
| 956 | IMPORTIN ALPHA EXPORT RECEPTOR |
| 957 | MRNA-NUCLEUS EXPORT |
| 958 | NUCLEOSOME ASSEMBLY CHAPERONE |
| 959 | DNA REPLICATION DEPENDENT NUCLEOSOME ASSEMBLY |
| 960 | MITOTIC CHROMOSOME |
| 961 | ANTIBACTERIAL RESPONSE PROTEIN |
| 962 | GLUTATHIONE PEROXIDASE |
| 963 | MRNA CATABOLISM |
| 964 | STEROID METABOLISM |
| 965 | C-4 METHYL STEROL OXIDASE |
| 966 | PEROXISOME TARGETING SIGNAL-2 RECEPTOR |
| 967 | ACTIN CROSS-LINKING |
| 968 | GLYCOSAMINOGLYCAN BIOSYNTHESIS |
| 969 | GRAM-NEGATIVE BACTERIAL BINDING |
| 970 | V-SNARE |
| 971 | ER-GOLGI TRANSPORT VESICLE |
| 972 | ACETYL-COA CARBOXYLASE |
| 973 | METHYLTRANSFERASE |
| 974 | PROTEIN METHYLATION |
| 975 | CYTOSKELETAL ANCHORING |
| 976 | MEIOTIC PROPHASE II |
| 977 | UDP-GLUCOSE METABOLISM |
| 978 | UDP-GLUCOSE 6-DEHYDROGENASE |
| 979 | UDP-GLUCURONATE BIOSYNTHESIS |
| 980 | URIDINE KINASE |
| 981 | PYRIMIDINE RIBONUCLEOTIDE BIOSYNTHESIS |
| 982 | GDP-MANNOSE 4,6-DEHYDRATASE |
| 983 | HEXOKINASE |
| 984 | GLUCOSE METABOLISM |
| 985 | AMILORIDE-SENSITIVE SODIUM CHANNEL |
| 986 | OSMOREGULATION AND EXCRETION |
| 987 | 2,4-DIENOYL-COA REDUCTASE (NADPH) |
| 988 | KATANIN |
| 989 | MICROTUBULE-SEVERING ATPASE |
| 990 | MICROTUBULE DEPOLYMERIZATION |
| 991 | 3-OXOACYL-[ACYL-CARRIER PROTEIN] REDUCTASE |
| 992 | MEMBRANE ASSOCIATED ACTIN BINDING |
| 993 | ADENINE PHOSPHORIBOSYLTRANSFERASE |
| 994 | TRNA BINDING |
| 995 | TRNA PROCESSING |
| 996 | ALANYL-TRNA BIOSYNTHESIS |
| 997 | ALCOHOL METABOLISM |
| 998 | ALDEHYDE DEHYDROGENASE (NAD+) |
| 999 | ALDEHYDE DEHYDROGENASE (NAD(P)+) |
| 1000 | ASPARAGINE--TRNA LIGASE |
| 1001 | CALCIUM STORAGE |
| 1002 | ENDOPLASMIC RETICULUM LUMEN |
| 1003 | HEMOGLOBIN |
| 1004 | FATTY ACID (OMEGA-1)-HYDROXYLASE |
| 1005 | INORGANIC DIPHOSPHATASE |
| 1006 | ISOCITRATE METABOLISM |
| 1007 | ISOCITRATE DEHYDROGENASE (NADP+) |
| 1008 | DIHYDROPYRIMIDINASE |
| 1009 | GLYCOGEN CATABOLISM |
| 1010 | COCHAPERONIN |
| 1011 | ECTODERM DEVELOPMENT |
| 1012 | LECTIN |
| 1013 | METHIONINE ADENOSYLTRANSFERASE |
| 1014 | ER RETENTION |
| 1015 | PROTEIN DISULFIDE ISOMERASE |
| 1016 | PROTEIN TRANSPORTER |
| 1017 | THIOREDOXIN PEROXIDASE |
| 1018 | UBIQUITIN |
| 1019 | POLY-UBIQUITIN |
| 1020 | CYTOKINE |
| 1021 | PROTEIN PROLINE HYDROXYLATION |
| 1022 | PROCOLLAGEN-PROLINE,2-OXOGLUTARATE-4- |
| | DIOXYGENASE |
| 1023 | DNA MODIFICATION |
| 1024 | PURINE-NUCLEOSIDE PHOSPHORYLASE |
| 1025 | PYRUVATE KINASE |
| 1026 | ARGINYL-TRNA BIOSYNTHESIS |
| 1027 | THIOREDOXIN |
| 1028 | PLASMA GLYCOPROTEIN |
| 1029 | POLYUBIQUITYLATION |
| 1030 | DNA REPAIR REGULATION |
| 1031 | CHLORIDE CHANNEL |
| 1032 | HEART DEVELOPMENT |
| 1033 | POTASSIUM CHANNEL REGULATOR |
| 1034 | LIGAND-GATED ION CHANNEL |
| 1035 | CYSTINE TRANSPORTER |
| 1036 | AMINO ACID TRANSPORTER |
| 1037 | DOLICHYL-DIPHOSPHOOLIGOSACCHARIDE-PROTEIN |
| | GLYCOSYLTRANSFERASE |
| 1038 | GALACTOSYLTRANSFERASE |
| 1039 | DEATH RECEPTOR INTERACTING PROTEIN |
| 1040 | RESPONSE TO WOUNDING |
| 1041 | GLUTAMINE--TRNA LIGASE |
| 1042 | GLUTAMINYL-TRNA BIOSYNTHESIS |
| 1043 | POTASSIUM CHANNEL |
| 1044 | 3-PHOSPHOINOSITIDE-DEPENDENT PROTEIN KINASE |
| 1045 | DNA DEPENDENT ADENOSINETRIPHOSPHATASE |
| 1046 | MICROVILLI |
| 1047 | ADENYLATE CYCLASE ACTIVATION |
| 1048 | ESTABLISHMENT OF CELL POLARITY |
| 1049 | CELL POLARITY |
| 1050 | PROTEASOME ATPASE |
| 1051 | CITRATE (SI)-SYNTHASE |
| 1052 | ISOVALERYL-COA DEHYDROGENASE |
| 1053 | COLD RESPONSE |
| 1054 | PERINUCLEAR SPACE |
| 1055 | MYOCYTE FUSION |
| 1056 | VOLTAGE-GATED CALCIUM CHANNEL |
| 1057 | LOW VOLTAGE-GATED CALCIUM CHANNEL |
| 1058 | INTERLEUKIN-5 RECEPTOR |
| 1059 | BETA-TUBULIN FOLDING |
| 1060 | TRANSLATION RELEASE FACTOR |
| 1061 | TRANSLATION TERMINATION FACTOR |
| 1062 | TRANSLATIONAL REGULATION, TERMINATION |
| 1063 | TRIOSEPHOSPHATE ISOMERASE |
| 1064 | NON-SELECTIVE VESICLE ASSEMBLY |
| 1065 | ARF SMALL MONOMERIC GTPASE |
| 1066 | FERTILIZATION |
| 1067 | ACETYL-COA C-ACETYLTRANSFERASE |
| 1068 | FATTY ACID BETA-OXIDATION |
| 1069 | LONG-CHAIN ACYL-COA DEHYDROGENASE |
| 1070 | ENERGY DERIVATION BY OXIDATION OF ORGANIC |
| | COMPOUNDS |
| 1071 | CARBONATE DEHYDRATASE |
| 1072 | MEMBRANE DIPEPTIDASE |
| 1073 | RETINOID BINDING |
| 1074 | ARGININE CATABOLISM |
| 1075 | GUANYL-NUCLEOTIDE EXCHANGE FACTOR |
| 1076 | FUMARATE HYDRATASE |
| 1077 | FUMARATE METABOLISM |
| 1078 | HEMOSTASIS |
| 1079 | GLUCOSE 6-PHOSPHATE UTILIZATION |
| 1080 | GLUCOSE-6-PHOSPHATE 1-DEHYDROGENASE |
| 1081 | CYSTEINE METABOLISM |
| 1082 | GLUTAMATE METABOLISM |
| 1083 | GLUTATHIONE BIOSYNTHESIS |
| 1084 | GLUTAMATE-CYSTEINE LIGASE |
| 1085 | AT DNA BINDING |
| 1086 | THIOREDOXIN REDUCTASE (NADPH) |
| 1087 | CHROMATIN ASSEMBLY/DISASSEMBLY |
| 1088 | KILLER ACTIVITY |
| 1089 | RESPIRATION |
| 1090 | PENTOSE-PHOSPHATE SHUNT, OXIDATIVE BRANCH |
| 1091 | HSP70/HSP90 ORGANIZING PROTEIN |
| 1092 | DNA FRAGMENTATION |
| 1093 | PHOSPHATIDYLETHANOLAMINE BINDING |
| 1094 | PROTEIN TYROSINE PHOSPHATASE ACTIVATOR |
| 1095 | PYRROLINE 5-CARBOXYLATE REDUCTASE |
| 1096 | SMOOTH ENDOPLASMIC RETICULUM |
| 1097 | SMALL UBIQUITIN-RELATED PROTEIN 1 CONJUGATION |
| 1098 | BRUSH BORDER |
| 1099 | CREATINE KINASE |
| 1100 | PENTOSE-PHOSPHATE SHUNT |
| 1101 | ALDEHYDE DEHYDROGENASE |
| 1102 | PERIPHERAL NERVOUS SYSTEM DEVELOPMENT |
| 1103 | ENOYL-COA HYDRATASE |
| 1104 | ACETYL-COA C-ACYLTRANSFERASE |
| 1105 | 3-HYDROXYACYL-COA DEHYDROGENASE |
| 1106 | NITRIC OXIDE SYNTHASE |
| 1107 | CLATHRIN ADAPTOR |
| 1108 | EXTRACELLULAR MATRIX GLYCOPROTEIN |
| 1109 | HIGH DENSITY LIPOPROTEIN BINDING |
| 1110 | CALCIUM CHANNEL |
| 1111 | GLUTATHIONE REDUCTASE (NADPH) |
| 1112 | HOMOPHILIC CELL ADHESION |
| 1113 | CALCIUM-INDEPENDENT CELL-CELL MATRIX ADHESION |
| 1114 | EMBRYONIC POLARITY |
| 1115 | MYO-INOSITOL: SODIUM SYMPORTER |
| 1116 | BEHAVIOR |
| 1117 | CANNABINOID RECEPTOR |
| 1118 | G-PROTEIN SIGNALLING, LINKED TO CYCLIC NUCLEOTIDE |
| | SECOND MESSENGER |
| 1119 | TIGHT JUNCTION |
| 1120 | MEMBRANE-ASSOCIATED PROTEIN WITH GUANYLATE |
| | KINASE ACTIVITY |
| 1121 | PERICENTRIOLAR MATERIAL |
| 1122 | FOCAL ADHESION KINASE |
| 1123 | SIGNAL COMPLEX FORMATION |
| 1124 | UREA CYCLE |
| 1125 | ARGININOSUCCINATE LYASE |
| 1126 | NUCLEOTIDE-SUGAR METABOLISM |
| 1127 | MEDIATOR COMPLEX |
| 1128 | FATTY-ACYL-COA SYNTHASE |
| 1129 | TRANSCRIPTION TERMINATION |
| 1130 | TRANSCRIPTION ELONGATION FACTOR COMPLEX |
| 1131 | POL II TRANSCRIPTION TERMINATION FACTOR |
| 1132 | CASPASE-3 |
| 1133 | POLY(ADP-RIBOSE) GLYCOHYDROLASE |
| 1134 | VOLTAGE-SENSITIVE CALCIUM CHANNEL |
| 1135 | TRNA GUANYLYLTRANSFERASE |
| 1136 | GLYCOSAMINOGLYCAN BINDING |
| 1137 | PROTEIN SERINE/THREONINE PHOSPHATASE |
| 1138 | RHO GTPASE ACTIVATOR |
| 1139 | CYTOSKELETON ORGANIZATION AND BIOGENESIS |
| 1140 | DNA LIGATION |
| 1141 | ALDEHYDE METABOLISM |
| 1142 | ALDO-KETO REDUCTASE |
| 1143 | CALCIUM-DEPENDENT PHOSPHOLIPID BINDING |
| 1144 | DIPHOSPHOINOSITOL POLYPHOSPHATE |
| | PHOSPHOHYDROLASE |
| 1145 | HETEROTRIMERIC G PROTEIN |
| 1146 | T CELL RECEPTOR |
| 1147 | POLY(U) BINDING |
| 1148 | ACETYL-COA METABOLISM |
| 1149 | CYTOSKELETAL ADAPTOR |
| 1150 | CAM-DEPENDENT CYCLIC-NUCLEOTIDE |
| | PHOSPHODIESTERASE |
| 1151 | CYCLIN-DEPENDENT PROTEIN KINASE INHIBITOR |
| 1152 | SERINE BIOSYNTHESIS |
| 1153 | PHOSPHOGLYCERATE DEHYDROGENASE |
| 1154 | TRYPSIN |
| 1155 | MITOTIC S PHASE |
| 1156 | G-PROTEIN-COUPLED RECEPTOR PHOSPHORYLATING |
| | PROTEIN KINASE |
| 1157 | OXIDATIVE PHOSPHORYLATION |
| 1158 | SMOOTH MUSCLE CONTRACTION REGULATION |
| 1159 | METHYLMALONYL-COA MUTASE |
| 1160 | CHEMOSENSATION AND RESPONSE |
| 1161 | CARBAMOYL-PHOSPHATE SYNTHASE (AMMONIA) |
| 1162 | NUCLEAR RNA-NUCLEUS EXPORT |
| 1163 | EMBRYO IMPLANTATION |
| 1164 | SMALL RIBOSOMAL SUBUNIT |
| 1165 | ENDOPLASMIC RETICULUM RECEPTOR |
| 1166 | DNA BENDING |
| 1167 | INTRACELLULAR |
| 1168 | PROTEIN SYNTHESIS INITIATION |
| 1169 | ION TRANSPORT |
| 1170 | GUANYL-NUCLEOTIDE RELEASING FACTOR |
| 1171 | SPLICEOSOME ASSEMBLY |
| 1172 | ADENYLATE CYCLASE |
| 1173 | CALCIUM/CALMODULIN-RESPONSIVE ADENYLATE |
| | CYCLASE |
| 1174 | NASCENT POLYPEPTIDE ASSOCIATION |
| 1175 | NASCENT POLYPEPTIDE-ASSOCIATED COMPLEX |
| 1176 | NONSENSE-MEDIATED MRNA DECAY |
| 1177 | ACYL-COA METABOLISM |
| 1178 | DEGRADATION OF CYCLIN |
| 1179 | CYCLIN SELECTIVE UBIQUITIN CONJUGATING ENZYME |
| 1180 | STEROID BINDING |
| 1181 | GLYCOSPHINGOLIPID METABOLISM |
| 1182 | MULTIDRUG TRANSPORTER |
| 1183 | ORGANIC ANION TRANSPORTER |
| 1184 | ASIALOGLYCOPROTEIN RECEPTOR |
| 1185 | NUCLEAR INNER MEMBRANE |
| 1186 | RIBONUCLEASE INHIBITOR |
| 1187 | CALCIUM/CALMODULIN-DEPENDENT PROTEIN KINASE |
| 1188 | NEUTRAL AMINO ACID TRANSPORT |
| 1189 | NEUTRAL AMINO ACID TRANSPORTER |
| 1190 | TRANSPORT |
| 1191 | CALPAIN |
| 1192 | PROTEIN PHOSPHATASE TYPE 2A CATALYST |
| 1193 | MEMORY |
| 1194 | MRNA SPLICE SITE SELECTION |
| 1195 | DNA TOPOISOMERASE I |
| 1196 | DRUG RESISTANCE |
| 1197 | KINESIN |
| 1198 | MICROTUBULE MOTOR |
| 1199 | NADPH--FERRIHEMOPROTEIN REDUCTASE |
| 1200 | MRNA BINDING, 3′ UTR |
| 1201 | COATOMER |
| 1202 | EUKARYOTIC TRANSLATION INITIATION FACTOR 2ALPHA |
| | KINASE |
| 1203 | HEAT RESPONSE |
| 1204 | RECEPTOR SIGNALLING PROTEIN SERINE/THREONINE |
| | KINASE |
| 1205 | NUCLEASE |
| 1206 | RNA MODIFICATION |
| 1207 | GOLGI VESICLE |
| 1208 | TYROSINE-TRNA LIGASE |
| 1209 | TYROSYL-TRNA BIOSYNTHESIS |
| 1210 | INTERLEUKIN-8 RECEPTOR LIGAND |
| 1211 | PURINE SALVAGE |
| 1212 | HYPOXANTHINE PHOSPHORIBOSYLTRANSFERASE |
| 1213 | HEME OXYGENASE (DECYCLIZING) |
| 1214 | TRNA MODIFICATION |
| 1215 | HISTONE MRNA METABOLISM |
| 1216 | MAPKKK CASCADE |
| 1217 | FGF RECEPTOR SIGNALLING PATHWAY |
| 1218 | FIBROBLAST GROWTH FACTOR RECEPTOR |
| 1219 | RAS GUANYL-NUCLEOTIDE EXCHANGE FACTOR |
| 1220 | PHOSPHOGLYCERATE MUTASE |
| 1221 | GLUTATHIONE TRANSFERASE |
| 1222 | HEAVY METAL RESISTANCE |
| 1223 | HEAVY METAL RESPONSE |
| 1224 | HEAVY METAL ION TRANSPORT |
| 1225 | COPPER, ZINC SUPEROXIDE DISMUTASE |
| 1226 | CYTOPLASMIC VESICLE |
| 1227 | CELL ADHESION INHIBITION |
| 1228 | RHO GDP-DISSOCIATION INHIBITOR |
| 1229 | CELL FATE SPECIFICATION |
| 1230 | CILIUM |
| 1231 | MORPHOGENESIS |
| 1232 | PHOSPHATIDYLINOSITOL TRANSPORTER |
| 1233 | COCHAPERONE |
| 1234 | POST-CHAPERONINE TUBULIN FOLDING PATHWAY |
| 1235 | PROTEINASE INHIBITOR |
| 1236 | HEAVY METAL ION TRANSPORTER |
| 1237 | LACTASE |
| 1238 | PHOSPHOFRUCTOKINASE |
| 1239 | GLYCOLYSIS REGULATION |
| 1240 | SEPTATE JUNCTION |
| 1241 | CELL-CELL ADHERENS JUNCTION |
| 1242 | INTERCELLULAR JUNCTION ASSEMBLY |
| 1243 | LACTOYLGLUTATHIONE LYASE |
| 1244 | HYDROXYMETHYLBILANE SYNTHASE |
| 1245 | DUTP PYROPHOSPHATASE |
| 1246 | PROTEIN PHOSPHATASE TYPE 2C |
| 1247 | INTERFERON-ALPHA/BETA RECEPTOR |
| 1248 | GLYCINE METABOLISM |
| 1249 | BILE ACID METABOLISM |
| 1250 | ARYLSULFATASE |
| 1251 | LYSOSOMAL TRANSPORT |
| 1252 | HYDROGEN-TRANSLOCATING V-TYPE ATPASE |
| 1253 | HEME TRANSPORTER |
| 1254 | GLYCOGEN PHOSPHORYLASE |
| 1255 | CREATINE TRANSPORTER |
| 1256 | NEUROTRANSMITTER UPTAKE |
| 1257 | CREATINE: SODIUM SYMPORTER |
| 1258 | EICOSANOID METABOLISM |
| 1259 | CALCIUM-DEPENDENT CYTOSOLIC PHOSPHOLIPASE A2 |
| 1260 | ENDORIBONUCLEASE |
| 1261 | ALDEHYDE OXIDASE |
| 1262 | XANTHINE DEHYDROGENASE |
| 1263 | OXYGEN AND RADICAL METABOLISM |
| 1264 | BLOOD GROUP ANTIGEN |
| 1265 | OXYGEN TRANSPORTER |
| 1266 | OXYGEN TRANSPORT |
| 1267 | NITRILASE |
| 1268 | RENIN |
| 1269 | DNA DAMAGE INDUCED PROTEIN PHOSPHORYLATION |
| 1270 | BLASTODERM SEGMENTATION |
| 1271 | MEMBRANE PROTEIN ECTODOMAIN PROTEOLYSIS |
| 1272 | SPINDLE MICROTUBULE |
| 1273 | ANTEROGRADE AXON CARGO TRANSPORT |
| 1274 | ORGANELLE ORGANIZATION AND BIOGENESIS |
| 1275 | GLYCIPAN |
| 1276 | EXO-ALPHA-SIALIDASE |
| 1277 | MANNOSYLTRANSFERASE |
| 1278 | LIPOPOLYSACCHARIDE BIOSYNTHESIS |
| 1279 | METABOLISM |
| 1280 | GPI-ANCHOR TRANSAMIDASE |
| 1281 | TROPOMYOSIN |
| 1282 | MUSCLE CONTRACTION REGULATION |
| 1283 | SYNAPTIC VESICLE |
| 1284 | NEUROTRANSMITTER RELEASE |
| 1285 | GOLGI STACK |
| 1286 | GLUTAREDOXIN |
| 1287 | ACID PHOSPHATASE |
| 1288 | DOPACHROME DELTA-ISOMERASE |
| 1289 | SUBSTRATE-BOUND CELL MIGRATION, CELL EXTENSION |
| 1290 | INTRACELLULAR COPPER DELIVERY |
| 1291 | CATHEPSIN D |
| 1292 | LEUKOTRIENE METABOLISM |
| 1293 | SPERMIDINE SYNTHASE |
| 1294 | POLYAMINE METABOLISM |
| 1295 | METHIONINE METABOLISM |
| 1296 | PROTEIN PHOSPHATASE INHIBITOR |
| 1297 | DEATH RECEPTOR LIGAND |
| 1298 | APOPTOTIC MITOCHONDRIAL CHANGES |
| 1299 | INDUCTION OF APOPTOSIS VIA DEATH DOMAIN RECEPTORS |
| 1300 | MOLECULAR_FUNCTION |
| 1301 | GLYCEROPHOSPHOLIPID METABOLISM |
| 1302 | 1-PHOSPHATIDOYLINOSITOL-4-PHOSPHATE KINASE |
| 1303 | MALATE DEHYDROGENASE |
| 1304 | CALCIUM-DEPENDENT CELL ADHESION |
| 1305 | THIOPURINE S-METHYLTRANSFERASE |
| 1306 | RHO SMALL MONOMERIC GTPASE |
| 1307 | ADP REDUCTION |
| 1308 | NUCLEOTIDE METABOLISM |
| 1309 | RIBONUCLEOSIDE DIPHOSPHATE CATABOLISM |
| 1310 | RIBONUCLEASE |
| 1311 | FATTY ACID BINDING |
| 1312 | MOLECULAR_FUNCTION UNKNOWN |
| 1313 | GERM CELL DEVELOPMENT |
| 1314 | REPRESSION OF SURVIVAL GENE PRODUCTS |
| 1315 | DIADENOSINE POLYPHOSPHATE CATABOLISM |
| 1316 | EXONUCLEASE |
| 1317 | UV PROTECTION |
| 1318 | DOUBLE-STRANDED DNA SPECIFIC |
| | EXODEOXYRIBONUCLEASE |
| 1319 | EGF RECEPTOR MODULATION |
| 1320 | AXONEMAL MOTOR |
| 1321 | AXONEMAL DYNEIN |
| 1322 | PROTEIN PHOSPHATASE |
| 1323 | INOSITOL/PHOSPHATIDYLINOSITOL PHOSPHATASE |
| 1324 | OSSIFICATION |
| 1325 | GLUCOSAMINE CATABOLISM |
| 1326 | GLUCOSAMINE-6-PHOSPHATE ISOMERASE |
| 1327 | MITOTIC METAPHASE/ANAPHASE TRANSITION |
| 1328 | SELENIUM BINDING |
| 1329 | G/T-MISMATCH-SPECIFIC THYMINE-DNA GLYCOSYLASE |
| 1330 | NON-SELECITVE VESICLE DOCKING |
| 1331 | INTRA GOLGI TRANSPORT |
| 1332 | INTER-GOLGI TRANSPORT VESICLE |
| 1333 | RAS SMALL MONOMERIC GTPASE |
| 1334 | PHOSPHOMEVALONATE KINASE |
| 1335 | PROTEASOME ACTIVATOR |
| 1336 | THYMIDYLATE KINASE |
| 1337 | PROSTAGLANDIN METABOLISM |
| 1338 | SINGLE-STRAND BREAK REPAIR |
| 1339 | PHOSPHATIDYLCHOLINE TRANSPORTER |
| 1340 | NEUROTRANSMITTER SYNTHESIS AND STORAGE |
| 1341 | TRANSALDOLASE |
| 1342 | SYNAPTONEMAL COMPLEX |
| 1343 | DIHYDROLIPOAMIDE DEHYDROGENASE |
| 1344 | CATABOLIC CARBOHYDRATE METABOLISM |
| 1345 | DEATH RECEPTOR ASSOCIATED FACTOR |
| 1346 | HYDROGEN TRANSPORTER |
| 1347 | PHENYLALANINE METABOLISM |
| 1348 | TETRAHYDROBIOPTERIN BIOSYNTHESIS |
| 1349 | 4A-HYDROXYTETRAHYDROBIOPTERIN DEHYDRATASE |
| 1350 | GALACTOKINASE |
| 1351 | GALACTOSE METABOLISM |
| 1352 | BIS(5′-NUCLEOSYL)-TETRAPHOSPHATASE (SYMMETRICAL) |
| 1353 | IONIC INSULATION OF NEURONS BY GLIAL CELLS |
| 1354 | TYPE 1 SERINE/THREONINE SPECIFIC PROTEIN |
| | PHOSPHATASE INHIBITOR |
| 1355 | BIOLOGICAL_PROCESS UNKNOWN |
| 1356 | CATHEPSIN H |
| 1357 | CASPASE-ACTIVATED DEOXYRIBONUCLEASE |
| 1358 | ACYLPHOSPHATASE |
| 1359 | ACYL-COA BINDING |
| 1360 | PROLYL OLIGOPEPTIDASE |
| 1361 | GROWTH FACTOR |
| 1362 | PHOSPHATIDYLINOSITOL-BISPHOSPHATASE |
| 1363 | ION CHANNEL INHIBITOR |
| 1364 | DIHYDROBIOPTERIN REDUCTION |
| 1365 | DIHYDROPTERIDINE REDUCTASE |
| 1366 | DIAZEPAM-BINDING INHIBITOR |
| 1367 | GALACTOSE BINDING LECTIN |
| 1368 | ORNITHINE METABOLISM |
| 1369 | ORNITHINE-OXO-ACID AMINOTRANSFERASE |
| 1370 | CATHEPSIN B |
| 1371 | BILE ACID TRANSPORTER |
| 1372 | CTP SYNTHASE |
| 1373 | SORBITOL METABOLISM |
| 1374 | UBIQUITIN-LIKE ACTIVATING ENZYME |
| 1375 | DIHYDROLIPOAMIDE S-ACETYLTRANSFERASE |
| 1376 | PHOSPHORYLASE |
| 1377 | GLUTAMATE CATABOLISM |
| 1378 | FRUCTOSE 2,6-BISPHOSPHATE METABOLISM |
| 1379 | FRUCTOSE-2,6-BISPHOSPHATE 2-PHOSPHATASE |
| 1380 | 3-BETA-HYDROXY-DELTA(5)-STEROID DEHYDROGENASE |
| 1381 | ALPHA DNA POLYMERASE:PRIMASE COMPLEX |
| 1382 | ACONITATE HYDRATASE |
| 1383 | MITOCHONDRIAL LARGE RIBOSOMAL-SUBUNIT |
| 1384 | MRNA EDITING |
| 1385 | CYCLOSPORIN A BINDING |
| 1386 | MEVALONATE TRANSPORT |
| 1387 | MEVALONATE TRANSPORTER |
| 1388 | MONOCARBOXYLIC ACID TRANSPORT |
| 1389 | MONOCARBOXYLIC ACID TRANSPORTER |
| 1390 | P-ELEMENT BINDING |
| 1391 | AMINOACYLASE |
| 1392 | ASPARTATE-TRNA LIGASE |
| 1393 | ASPARTYL-TRNA BIOSYNTHESIS |
| 1394 | POLYPEPTIDE N-ACETYLGALACTOSAMINYLTRANSFERASE |
| 1395 | COLLAGEN |
| 1396 | HYDROGEN/POTASSIUM-EXCHANGING ATPASE |
| 1397 | SARCOGLYCAN COMPLEX |
| 1398 | FUCOSYLTRANSFERASE |
| 1399 | AMINOPEPTIDASE |
| 1400 | UDP-GLUCOSE 4-EPIMERASE |
| 1401 | TRANSPORTIN |
| 1402 | METHIONYL AMINOPEPTIDASE |
| 1403 | DIPEPTIDYL-PEPTIDASE |
| 1404 | PITRILYSIN |
| 1405 | 2′,3′-CYCLIC NUCLEOTIDE 3′-PHOSPHODIESTERASE |
| 1406 | NUCLEAR OUTER MEMBRANE |
| 1407 | IMP DEHYDROGENASE |
| 1408 | PEPTIDE METABOLISM |
| 1409 | SECRETORY VESICLE MEMBRANE |
| 1410 | GLYCINE--TRNA LIGASE |
| 1411 | HETEROTRIMERIC G-PROTEIN GTPASE, ALPHA SUBUNIT |
| 1412 | ARF GTPASE ACTIVATOR |
| 1413 | PEROXISOMAL MATRIX |
| 1414 | VERY LONG CHAIN FATTY ACID METABOLISM |
| 1415 | ALPHA-GLUCOSIDASE |
| 1416 | PHAGOCYTOSIS |
| 1417 | CREATINE BIOSYNTHESIS |
| 1418 | GLYCINE AMIDINOTRANSFERASE |
| 1419 | MAP KINASE |
| 1420 | CALCIUM-ACTIVATED POTASSIUM CHANNEL |
| 1421 | BLEOMYCIN HYDROLASE |
| 1422 | AMINOBUTYRATE CATABOLISM |
| 1423 | SUCCINATE-SEMIALDEHYDE DEHYDROGENASE |
| 1424 | LYSOSOMAL MEMBRANE |
| 1425 | PHAGOSOME FORMATION |
| 1426 | DEBRANCHING ENZYME |
| 1427 | GLUTAMATE DEHYDROGENASE |
| 1428 | SPHINGOLIPID METABOLISM |
| 1429 | SERINE C-PALMITOYLTRANSFERASE |
| 1430 | ENDOSOME TO LYSOSOME TRANSPORT |
| 1431 | CELL GROWTH AND/OR MAINTENANCE |
| 1432 | NAD(P)(+) TRANSHYDROGENASE (B-SPECIFIC) |
| 1433 | MAJOR HISTOCOMPATIBILITY PEPTIDE TRANSPORTER |
| 1434 | AMINOGLYCAN BIOSYNTHESIS |
| 1435 | ACETYLGLUCOSAMINYLTRANSFERASE |
| 1436 | UBIQUINOL-CYTOCHROME-C REDUCTASE |
| 1437 | ADENYLATE CYCLASE INHIBITION |
| 1438 | GLUCONEOGENESIS |
| 1439 | DICARBOXYLIC ACID TRANSPORT |
| 1440 | DICARBOXYLIC ACID TRANSPORTER |
| 1441 | CARRIER |
| 1442 | NON-SELECTIVE VESICLE FUSION |
| 1443 | MITOCHONDRIAL CITRATE TRANSPORT |
| 1444 | SODIUM: DICARBOXYLATE/TRICARBOXYLATE |
| | COTRANSPORTER |
| 1445 | SUBTILISIN |
| 1446 | PROPROTEIN CONVERTASE 2 |
| 1447 | SERINE--TRNA LIGASE |
| 1448 | METALLOEXOPEPTIDASE |
| 1449 | HOLOCYTOCHROME C SYNTHASE |
| 1450 | AXON GUIDANCE |
| 1451 | PROTEIN-MEMBRANE TARGETING |
| 1452 | COMPLEMENT COMPONENT |
| 1453 | COMPLEMENT ACTIVATION |
| 1454 | OXOGLUTARATE DEHYDROGENASE (LIPOAMIDE) |
| 1455 | POTASSIUM: CHLORIDE SYMPORTER |
| 1456 | GLYCINE CATABOLISM |
| 1457 | GLYCINE DEHYDROGENASE (DECARBOXYLATING) |
| 1458 | OLIGOSACCHARIDE METABOLISM |
| 1459 | MANNOSYL-OLIGOSACCHARIDE 1,2-ALPHA-MANNOSIDASE |
| 1460 | N-GLYCAN PROCESSING |
| 1461 | UTP--GLUCOSE-1-PHOSPHATE URIDYLYLTRANSFERASE |
| 1462 | O-METHYLTRANSFERASE |
| 1463 | SOLUTE:CATION SYMPORTER |
| 1464 | CYTOCHROME C |
| 1465 | OXOGLUTARATE/MALATE ANTIPORTER |
| 1466 | 3′-5′ EXODEOXYRIBONUCLEASE |
| 1467 | CYTOCHROME B |
| 1468 | PYRUVATE METABOLISM |
| 1469 | MALATE DEHYDROGENASE (DECARBOXYLATING) |
| 1470 | PROTEIN KINASE C |
| 1471 | CASPASE ACTIVATION VIA CYTOCHROME C |
| 1472 | UDP-GLUCOSE:GLYCOPROTEIN GLUCOSYLTRANSFERASE |
| 1473 | ACTIVATION OF MAP/ERK KINASE KINASE |
| 1474 | CATALASE |
| 1475 | INSOLUBLE FRACTION |
| 1476 | MITOCHONDRIAL TRANSPORT |
| 1477 | GOLGI LUMEN |
| 1478 | ENDOCYTOTIC TRANSPORT VESICLE |
| 1479 | SODIUM DEPENDENT MULTIVITAMIN TRANSPORTER |
| 1480 | POLY-GLUTAMINE TRACT BINDING |
| 1481 | TRANSMEMBRANE RECEPTOR PROTEIN SERINE/THREONINE |
| | KINASE |
| 1482 | TRANSMEMBRANE RECEPTOR PROTEIN SERINE/THREONINE |
| | KINASE SIGNALLING PATHWAY |
| 1483 | DRUG TRANSPORTER |
| 1484 | STEROID DELTA-ISOMERASE |
| 1485 | PHOSPHORYLASE KINASE REGULATOR |
| 1486 | FERREDOXIN--NADP(+) REDUCTASE |
| 1487 | PROFILIN BINDING |
| 1488 | ATP-DEPENDENT PEPTIDASE |
| 1489 | EPOXIDE HYDROLASE |
| 1490 | LEUKOTRIENE-A4 HYDROLASE |
| 1491 | MANNOSE METABOLISM |
| 1492 | MANNOSE-6-PHOSPHATE ISOMERASE |
| 1493 | CELL WALL |
| 1494 | N-ACETYLGLUCOSAMINE METABOLISM |
| 1495 | N-ACETYLMANNOSAMINE METABOLISM |
| 1496 | CENTROMERE/KINETOCHORE COMPLEX MATURATION |
| 1497 | 3-HYDROXYISOBUTYRYL-COA HYDROLASE |
| 1498 | PYRUVATE DEHYDROGENASE (LIPOAMIDE) |
| 1499 | COPROPORPHYRINOGEN OXIDASE |
| 1500 | EXOPEPTIDASE |
| 1501 | GAMMA-GLUTAMYL HYDROLASE |
| 1502 | DOUBLE-STRANDED RNA ADENOSINE DEAMINASE |
| 1503 | ETHANOL OXIDATION |
| 1504 | ETHANOL METABOLISM |
| 1505 | ALCOHOL DEHYDROGENASE |
| 1506 | ALCOHOL DEHYDROGENASE, ZINC-DEPENDENT |
| 1507 | ANTIMICROBIAL RESPONSE PROTEIN |
| 1508 | BRANCHED-CHAIN AMINO ACID AMINOTRANSFERASE |
| 1509 | BRANCHED CHAIN FAMILY AMINO ACID BIOSYNTHESIS |
| 1510 | SUCCINYL-COA METABOLISM |
| 1511 | 3-OXOACID COA-TRANSFERASE |
| 1512 | PHOSPHATIDYLINOSITOL BIOSYNTHESIS |
| 1513 | PROTEIN PHOSPHATASE TYPE 1 REGULATOR |
| 1514 | SERINE METABOLISM |
| 1515 | PHOSPHOSERINE PHOSPHATASE |
| 1516 | MUTAGENESIS |

A resulting entry for a peptide sequence based on these criteria will have the following format: SEQ ID NO of peptide entry|Numeric code corresponding to cell type and HLA type|SEQ ID NOs of source protein reference(s)|source protein symbol(s)|Numeric keys corresponding to biological classification(s). This ordering corresponds to Criteria 1|Criteria 2|Criteria 3|Criteria 4|Criteria 5.

### Example 2

### Isolation and Characterization of Global Peptide Tags (GPT)

This example describes the use of enzymatic or chemical digestion strategies to reduce proteins of a complex mixture into peptides. These peptides are called Global Peptide Tags (GPT). The peptides were separated and fractionated by multiple modes of chromatography and ultimately sequenced by liquid chromatography on-line with tandem mass spectrometry (LC/MS/MS).

Prior to digestion, all proteins of a sample were denatured using high concentrations of chemical denaturants (such as 6-8 M Urea or 6-8 M guanidine hydrochloride), elevated temperature, or a combination of both chemical denaturants and elevated temperature. Additionally, reactive thiol groups were typically reduced by the action of dithiothreitol (DTT) or Tris[2-carboxyethylphosphine] hydrochloride (TCEP) at a molar concentration of 25 to 50 times greater than that of the total protein concentration, and alkylated with an alkylating agent (at a molar concentration of 25 to 50 times greater than that of the total protein concentration) such as iodoacetamide or iodoacetic acid. The reaction was typically carried out at room temperature and in the dark.

Two rounds of reduction and alkylation were usually used for each protein mixture to ensure complete reduction and alkylation of reactive thiol groups. Solutions were typically made in a 50-100 mM ammonium bicarbonate solution at an approximate pH of 8.2. Subsequently, the reduced and alkylated protein mixture was concentrated to a volume of approximately 50-100 uL under vacuum in a Speedvac™ centrifugal concentrator (ThermoSavant Scientific) used at ambient temperature. This process also removes the majority of the excess DTT that is used to quench the final alkylation reaction. Following concentration, the resultant solution was re-diluted with the ammonium bicarbonate solution and an enzyme was added at a weight to weight ratio of 1 part enzyme to 25-40 parts protein. Those protein mixtures that were chemically denatured were diluted with the ammonium bicarbonate solution to reduce the concentration of the chemical denaturant to less than 1 M. The enzyme was also dissolved in the ammonium bicarbonate solution. Enzymes that have been used to generate peptides from a complex protein mixture include trypsin and lysine endopeptidase. All enzymatic digestions were carried out overnight (typically 18 to 26 hours) at a temperature of 37° C. After protein digestion, enzymes were deactivated using 10% acetic acid solution, and peptides were separated from undigested protein and isolated by ultra filtration using either a 3 kDa or 5 kDa spin filtration device. Alternatively, total protein digest are generated chemically using cyanogen bromide. Again, peptides were isolated from undigested proteins by ultra filtration.

Peptide-rich solutions were separated by multiple modes of chromatography. The first mode was usually strong cation exchange (SCX) using a stationary phase such as polyethylaspartamide (from PolyLC Inc.) and an aqueous mobile phase that was modified with acetonitrile (5-15% v/v) and developed a salt gradient from 0 to 1 M salt to elute the adsorbed peptides. Each of the peptide-rich fractions that was isolated by SCX chromatography was further separated and fractionated using a C18 reversed phase microbore (1 mm id) column and mobile phases that were modified with trifluoroacetic acid and developed an acetonitrile gradient. Peptide-rich fractions that were isolated by reversed phase chromatography were subjected to on-line LC/MS/MS using a further dimension of reversed phase chromatography. Peptide sequence elucidation was by database searching raw MS/MS spectra against publicly available protein sequence databases.

Table 1 contains the sequences of a series of EPTs and GPTs identified as described in Examples 1 and 2. The conventions detailed in Example 1 are used to describe EPTs and GPTs, with the exception of “HLA source” which does not apply to GPTs.

TABLE 1
SEQ ID NO: 1|2|SEQ ID NO: 236, SEQ ID NO: 237, SEQ ID NO: 238, SEQ ID NO: 239, SEQ ID NO: 240, SEQ
ID NO: 241, SEQ ID NO: 242, SEQ ID NO: 243, SEQ ID NO: 244, SEQ ID NO: 245, SEQ ID NO: 246|GRIN2A,
NMDAR2A|22, 27, 28, 29, 30, 31, 32, 33, 34|
SEQ ID NO: 2|2|SEQ ID NO: 247, SEQ ID NO: 248, SEQ ID NO: 249|EFP, TRIM25, Z147, ZNF147|23, 119, 120|
SEQ ID NO: 3|2|SEQ ID NO: 250, SEQ ID NO: 251, SEQ ID NO: 252, SEQ ID NO: 253, SEQ ID NO: 254, SEQ
ID NO: 255|GPM6, GPM6A, M6A|31, 107|
SEQ ID NO: 4|2, 10|SEQ ID NO: 256, SEQ ID NO: 257, SEQ ID NO: 258, SEQ ID NO: 259, SEQ ID NO: 260,
SEQ ID NO: 261, SEQ ID NO: 262, SEQ ID NO: 263, SEQ ID NO: 264|CD27L, CD27LG, CD70, KI-24 ANTIGEN,
TNFSF7|31, 56, 57, 89, 120, 179, 387, 388, 389|
SEQ ID NO: 5|2, 10|SEQ ID NO: 265, SEQ ID NO: 266, SEQ ID NO: 267, SEQ ID NO: 268, SEQ ID NO: 269,
SEQ ID NO: 270, SEQ ID NO: 271, SEQ ID NO: 272, SEQ ID NO: 273, SEQ ID NO: 274, SEQ ID NO: 275, SEQ
ID NO: 276, SEQ ID NO: 277, SEQ ID NO: 278, SEQ ID NO: 279|CD132, IL2RG, IMD4, SCIDX, SCIDX1|31, 34,
57, 89, 127, 129, 332, 402, 403, 404|
SEQ ID NO: 6|2|SEQ ID NO: 280, SEQ ID NO: 281, SEQ ID NO: 282, SEQ ID NO: 283|BR, CD49B, ITGA2|17,
18, 34, 56, 154, 186, 187, 200, 345, 405, 406, 407, 408, 409, 410|
SEQ ID NO: 7|2|SEQ ID NO: 284, SEQ ID NO: 285, SEQ ID NO: 286, SEQ ID NO: 287, SEQ ID NO: 288, SEQ
ID NO: 289, SEQ ID NO: 290, SEQ ID NO: 291, SEQ ID NO: 292, SEQ ID NO: 293, SEQ ID NO: 294|ERBB4,
HER4|12, 31, 34, 36, 56, 57, 58, 59, 411|
SEQ ID NO: 8|9|SEQ ID NO: 295, SEQ ID NO: 296, SEQ ID NO: 297, SEQ ID NO: 298, SEQ ID NO: 299|PA26|
8, 55, 56, 371, 384, 465|
SEQ ID NO: 9| 9, 24| SEQ ID NO: 300, SEQ ID NO: 301, SEQ ID NO: 302, SEQ ID NO: 303, SEQ ID NO: 304,
SEQ ID NO: 305, SEQ ID NO: 306, SEQ ID NO: 307, SEQ ID NO: 308, SEQ ID NO: 309, SEQ ID NO: 310, SEQ
ID NO: 311|CCND2, KIAK0002|62, 136, 137|
SEQ ID NO: 10|9|SEQ ID NO: 312, SEQ ID NO: 313, SEQ ID NO: 314, SEQ ID NO: 315, SEQ ID NO: 316, SEQ
ID NO: 317, SEQ ID NO: 318, SEQ ID NO: 319, SEQ ID NO: 320, SEQ ID NO: 321, SEQ ID NO: 322, SEQ ID
NO: 323, SEQ ID NO: 324, SEQ ID NO: 325, SEQ ID N0:326|ADPRT, ADPRT1, PADPRT-1, PARP, PARP-1, PPOL|3,
7, 8, 10, 12, 13, 18, 53, 60, 179, 335, 336, 337|
SEQ ID NO: 11|9, 14|SEQ ID NO: 327, SEQ ID NO: 328, SEQ ID NO: 329, SEQ ID NO: 330, SEQ ID NO: 331|
ETFB|44, 481, 482|
SEQ ID NO: 12|9|SEQ ID NO: 332, SEQ ID NO: 333, SEQ ID NO: 334, SEQ ID NO: 335, SEQ ID NO: 336, SEQ
ID NO: 337, SEQ ID NO: 338, SEQ ID NO: 339|DAGK5, DGKZ, HDGKZETA|8, 12, 89, 98, 362, 487|
SEQ ID NO: 13|9, 13, 24, 28|SEQ ID NO: 340, SEQ ID NO: 341, SEQ ID NO: 342, SEQ ID NO: 343, SEQ ID
NO: 344, SEQ ID NO: 345, SEQ ID NO: 346|PDCD5, TFAR19|179, 211|
SEQ ID NO: 14|9|SEQ ID NO: 347, SEQ ID NO: 348, SEQ ID NO: 349, SEQ ID NO: 350, SEQ ID NO: 351, SEQ
ID NO: 352, SEQ ID NO: 353, SEQ ID NO: 354, SEQ ID NO: 355, SEQ ID NO: 356, SEQ ID NO: 357, SEQ ID
NO: 358, SEQ ID NO: 359, SEQ ID NO: 360|ACTR, AIB1, CAGH16, CTG26, NCOA3, P/CIP, RAC3, TNRC14, TNRC16,
TRAM-1|8, 10, 17, 36, 89, 118, 151|
SEQ ID NO: 15|9, 13|SEQ ID NO: 361, SEQ ID NO: 362, SEQ ID NO: 363, SEQ ID NO: 364, SEQ ID NO: 365,
SEQ ID NO: 366, SEQ ID NO: 367, SEQ ID NO: 368, SEQ ID NO: 369, SEQ ID NO: 370, SEQ ID NO: 371, SEQ
ID NO: 372, SEQ ID NO: 373|ASH2L, ASH2L1, ASH2L2|7, 8, 10, 14, 53, 54|
SEQ ID NO: 16|9|SEQ ID NO: 374, SEQ ID NO: 375, SEQ ID NO: 376, SEQ ID NO: 377, SEQ ID NO: 378, SEQ
ID NO: 379, SEQ ID NO: 380, SEQ ID NO: 381, SEQ ID NO: 382, SEQ ID NO: 383, SEQ ID NO: 384|CD53,
MOX44|31, 56, 89, 129, 141, 154|
SEQ ID NO: 17|9|SEQ ID NO: 385, SEQ ID NO: 386, SEQ ID NO: 387, SEQ ID NO: 388, SEQ ID NO: 389|
RGS14|17, 239, 242, 243, 441|
SEQ ID NO: 18|9, 13|SEQ ID NO: 390, SEQ ID NO: 391, SEQ ID NO: 392, SEQ ID NO: 393, SEQ ID NO: 394|
DDX9, LKP, NDHII, RHA|7, 8, 47, 67, 74, 84, 228, 301, 513|
SEQ ID NO: 19|9|SEQ ID NO: 395, SEQ ID NO: 396, SEQ ID NO: 397, SEQ ID NO: 398|AFP, TRIM26, ZNF173|
3, 7, 149|
SEQ ID NO: 20|9|SEQ ID NO: 399, SEQ ID NO: 400, SEQ ID NO: 401, SEQ ID NO: 402, SEQ ID NO: 403, SEQ
ID NO: 404|RPA2|7, 65, 452, 545, 546|
SEQ ID NO: 21|9|SEQ ID NO: 405, SEQ ID NO: 406, SEQ ID NO: 407, SEQ ID NO: 408, SEQ ID NO: 409, SEQ
ID NO: 410, SEQ ID NO: 411|CDC18L, CDC6, HSCDC18, HSCDC6|8, 55, 56, 65, 67, 221, 529, 547, 548, 549, 550|
SEQ ID NO: 22|9|SEQ ID NO: 412, SEQ ID NO: 413, SEQ ID NO: 414, SEQ ID NO: 415, SEQ ID NO: 416, SEQ
ID NO: 417, SEQ ID NO: 418, SEQ ID NO: 419, SEQ ID NO: 420, SEQ ID NO: 421, SEQ ID NO: 422|CBL, CBL2,
CBLB|23, 36, 89, 140, 149, 329|
SEQ ID NO: 23|9|SEQ ID NO: 423, SEQ ID NO: 424, SEQ ID NO: 425, SEQ ID NO: 426, SEQ ID NO: 427, SEQ
ID NO: 428, SEQ ID NO: 429, SEQ ID NO: 430, SEQ ID NO: 431, SEQ ID NO: 432|KIAA0898, MUL|
SEQ ID NO: 24|7, 9|SEQ ID NO: 433, SEQ ID NO: 434, SEQ ID NO: 435, SEQ ID NO: 436, SEQ ID NO: 437,
SEQ ID NO: 438, SEQ ID NO: 439, SEQ ID NO: 440, SEQ ID NO: 441, SEQ ID NO: 442, SEQ ID NO: 443, SEQ
ID NO: 444, SEQ ID NO: 445|PPP2R1A, PPP2R1B|2, 137, 558, 559|
SEQ ID NO: 25|9|SEQ ID NO: 446, SEQ ID NO: 447, SEQ ID NO: 448, SEQ ID NO: 449, SEQ ID NO: 450, SEQ
ID NO: 451, SEQ ID NO: 452, SEQ ID NO: 453 (HUMNDME, ME1|45, 110, 158, 303, 561, 562, 563|
SEQ ID NO: 26|9|SEQ ID NO: 454, SEQ ID NO: 455, SEQ ID NO: 456, SEQ ID NO: 457, SEQ ID NO: 458, SEQ
ID NO: 459, SEQ ID NO: 460, SEQ ID NO: 461, SEQ ID NO: 462, SEQ ID N: 463|M11S1|31|
SEQ ID NO: 27|9|SEQ ID NO: 464, SEQ ID NO: 465, SEQ ID NO: 466, SEQ ID NO: 467, SEQ ID NO: 468, SEQ
ID NO: 469, SEQ ID NO: 470, SEQ ID NO: 471, SEQ ID NO: 472, SEQ ID NO: 473|BAL, BSSL, CEL, IRF4, LSIRF,
MUM1|7, 36, 54, 76, 568|
SEQ ID NO: 28|9|SEQ ID NO: 474, SEQ ID NO: 475, SEQ ID NO: 476, SEQ ID NO: 477, SEQ ID NO: 478, SEQ
ID NO: 479, SEQ ID NO: 480, SEQ ID NO: 481, SEQ ID NO: 482, SEQ ID NO: 483, SEQ ID NO: 484, SEQ ID
NO: 485|MDH2|
SEQ ID NO: 29|9|SEQ ID NO: 486, SEQ ID NO: 487, SEQ ID NO: 488, SEQ ID NO: 489, SEQ ID NO: 490, SEQ
ID NO: 491, SEQ ID NO: 492, SEQ ID NO: 493, SEQ ID NO: 494, SEQ ID NO: 495, SEQ ID NO: 496, SEQ ID
NO: 497|PCNA|3, 7, 8, 56, 57, 60, 61, 62, 63, 64, 65, 66|
SEQ ID NO: 30|9|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 505, SEQ ID NO: 506, SEQ ID NO: 507, SEQ ID NO: 508, SEQ ID
NO: 509, SEQ ID NO: 510|4F2, 4F2HC, CD98, MDU1, NACAE, SLC3A2|
SEQ ID NO: 31|9|SEQ ID NO: 511, SEQ ID NO: 512, SEQ ID NO: 513, SEQ ID NO: 514, SEQ ID NO: 515, SEQ
ID NO: 516|PK1.3|2|
SEQ ID NO: 32|9|SEQ ID NO: 517, SEQ ID NO: 518, SEQ ID NO: 519, SEQ ID NO: 520, SEQ ID NO: 521, SEQ
ID NO: 522, SEQ ID NO: 523, SEQ ID NO: 524, SEQ ID NO: 525, SEQ ID NO: 526|JNKK2, MAP2K7, MAPKK7,
MKK7, PRKMK7|12, 89, 384, 579, 586|
SEQ ID NO: 33|9|SEQ ID NO: 527, SEQ ID NO: 528, SEQ ID NO: 529, SEQ ID NO: 530, SEQ ID NO: 531, SEQ
ID NO: 532, SEQ ID NO: 533, SEQ ID NO: 534, SEQ ID NO: 535, SEQ ID NO: 536, SEQ ID NO: 537, SEQ ID
NO: 538, SEQ ID NO: 539, SEQ ID NO: 540, SEQ ID NO: 541, SEQ ID NO: 542, SEQ ID NO: 543, SEQ ID
NO: 544, SEQ ID NO: 545, SEQ ID NO: 546, SEQ ID NO: 547|MYC|4, 8, 10, 17, 18, 23, 56, 57, 151, 371, 590|
SEQ ID NO: 34|9|SEQ ID NO: 548, SEQ ID NO: 549, SEQ ID NO: 550, SEQ ID NO: 551, SEQ ID NO: 552, SEQ
ID NO: 553, SEQ ID NO: 554|DJ196E23.2, HTATSF1, TAT-SF1|8, 10, 54, 151, 231, 556, 593|
SEQ ID NO: 35|9, 13|SEQ ID NO: 555, SEQ ID NO: 556, SEQ ID NO: 557, SEQ ID NO: 558, SEQ ID NO: 559,
SEQ ID NO: 560, SEQ ID NO: 561, SEQ ID NO: 562, SEQ ID NO: 563, SEQ ID NO: 564, SEQ ID NO: 565, SEQ
ID NO: 566, SEQ ID NO: 567|BRAF|12, 87, 104, 176, 409|
SEQ ID NO: 36|9|SEQ ID NO: 568, SEQ ID NO: 569, SEQ ID NO: 570, SEQ ID NO: 571, SEQ ID NO: 572, SEQ
ID NO: 573, SEQ ID NO: 574|DNAPK, DNPK1, HYRC1, PRKDC, XRCC7|12, 60, 104, 129, 165, 166, 167|
SEQ ID NO: 37|9, 13|SEQ ID NO: 575, SEQ ID NO: 576, SEQ ID NO: 577, SEQ ID NO: 578, SEQ ID NO: 579,
SEQ ID NO: 580, SEQ ID NO: 581, SEQ ID NO: 582|EPS15R|
SEQ ID NO: 38|9, 24|SEQ ID NO: 583, SEQ ID NO: 584, SEQ ID NO: 585, SEQ ID NO: 586, SEQ ID NO: 587,
SEQ ID NO: 588|HEC|8, 35, 174, 601|
SEQ ID NO: 39|9|SEQ ID NO: 589, SEQ ID NO: 590, SEQ ID NO: 591, SEQ ID NO: 592, SEQ ID NO: 593, SEQ
ID NO: 594|14.1, CD179B, IGI, IGL5, IGLJ1, IGLL, IGLL1, IGO, IGVPB, POR1, VPREB1|47, 149, 161, 201, 306,
604, 605, 606|
SEQ ID NO: 40|9|SEQ ID NO: 595, SEQ ID NO: 596, SEQ ID NO: 597, SEQ ID NO: 598, SEQ ID NO: 599, SEQ
ID NO: 600, SEQ ID NO: 601, SEQ ID NO: 602, SEQ ID NO: 603, SEQ ID NO: 604, SEQ ID NO: 605, SEQ ID
NO: 606, SEQ ID NO: 607, SEQ ID NO: 608, SEQ ID NO: 609, SEQ ID NO: 610|ENO1, EN01L1, MBP-1, MPB1, NNE,
PPH|3, 7, 9, 10, 18, 23, 24, 25, 26|
SEQ ID NO: 41|9|SEQ ID NO: 611, SEQ ID NO: 612, SEQ ID NO: 613, SEQ ID NO: 614, SEQ ID NO: 615, SEQ
ID NO: 616, SEQ ID NO: 617, SEQ ID NO: 618|ITGB7|34, 186, 405, 408|
SEQ ID NO: 42|9|SEQ ID NO: 619, SEQ ID NO: 620, SEQ ID NO: 621, SEQ ID NO: 622, SEQ ID NO: 623, SEQ
ID NO: 624, SEQ ID NO: 625, SEQ ID NO: 626, SEQ ID NO: 627, SEQ ID NO: 628, SEQ ID NO: 629, SEQ ID
NO: 630|PCCB|42, 71, 111, 615, 616|
SEQ ID NO: 43|9|SEQ ID NO: 631, SEQ ID NO: 632, SEQ ID NO: 633, SEQ ID NO: 634, SEQ ID NO: 635, SEQ
ID NO: 636, SEQ ID NO: 637, SEQ ID NO: 638, SEQ ID NO: 639, SEQ ID NO: 640, SEQ ID NO: 641, SEQ ID
NO: 642, SEQ ID NO: 643, SEQ ID NO: 644, SEQ ID NO: 645, SEQ ID NO: 646, SEQ ID NO: 647, SEQ ID
NO: 648|CBP, CREBBP, EP300, P300, RSTS, RTS|8, 10, 14, 17, 23, 53, 67, 89, 106, 107, 118, 135, 332, 557|
SEQ ID NO: 44|9|SEQ ID NO: 649, SEQ ID NO: 650, SEQ ID NO: 651, SEQ ID NO: 652, SEQ ID NO: 653|HGFR,
MET|12, 31, 34, 36, 56, 57, 89, 631|
SEQ ID NO: 45|9|SEQ ID NO: 654, SEQ ID NO: 655, SEQ ID NO: 656, SEQ ID NO: 657, SEQ ID NO: 658, SEQ
ID NO: 659, SEQ ID NO: 660, SEQ ID NO: 661, SEQ ID NO: 662, SEQ ID NO: 663, SEQ ID NO: 664, SEQ ID
NO: 665|CDHF12, HSCR1, MEN2A, MEN2B, MTC1, PTC, RET|12, 34, 36, 87, 89, 92, 107, 385, 448, 634|
SEQ ID NO: 46|4|SEQ ID NO: 666, SEQ ID NO: 667, SEQ ID NO: 668, SEQ ID NO: 669, SEQ ID NO: 670, SEQ
ID NO: 671, SEQ ID NO: 672, SEQ ID NO: 673|DEC-205, GP200-MR6, LY75|31, 34, 127, 129, 298, 385, 675|
SEQ ID NO: 47|1|SEQ ID NO: 674, SEQ ID NO: 675, SEQ ID NO: 676, SEQ ID NO: 677, SEQ ID NO: 678, SEQ
ID NO: 679, SEQ ID NO: 680, SEQ ID NO: 681|CD99, MIC2, MIC2X, MIC2Y|31, 67, 129, 154, 186, 187, 717|
SEQ ID NO: 48|1|SEQ ID NO: 682, SEQ ID NO: 683, SEQ ID NO: 684, SEQ ID NO: 685, SEQ ID NO: 686, SEQ
ID NO: 687, SEQ ID NO: 688|CD71, TFR, TFRC|31, 331, 590, 720, 721, 722|
SEQ ID NO: 49|1|SEQ ID NO: 689, SEQ ID NO: 690, SEQ ID NO: 691, SEQ ID NO: 692, SEQ ID NO: 693, SEQ
ID NO: 694, SEQ ID NO: 695, SEQ ID N0:696|CD49D, ITGA4|31, 34, 186, 408|
SEQ ID NO: 50|10|SEQ ID NO: 256, SEQ ID NO: 257, SEQ ID NO: 258, SEQ ID NO: 259, SEQ ID NO: 260, SEQ
ID NO: 261, SEQ ID NO: 262, SEQ ID NO: 263, SEQ ID NO: 264|CD27L, CD27LG, CD70, KI-24 ANTIGEN,
TNFSF7|31, 56, 57, 89, 120, 179, 387, 388, 389|
SEQ ID NO: 51|10|SEQ ID NO: 697, SEQ ID NO: 698, SEQ ID NO: 699, SEQ ID NO: 700, SEQ ID NO: 701, SEQ
ID NO: 702, SEQ ID NO: 703|CUBN, GP280, IFCR, MGA1|34, 242, 253, 342, 385, 461, 755, 756|
SEQ ID NO: 52|10|SEQ ID NO: 704, SEQ ID NO: 705, SEQ ID NO: 706, SEQ ID NO: 707|GABRD|22, 29, 31, 33,
34, 89, 775|
SEQ ID NO: 53|10|SEQ ID NO: 708, SEQ ID NO: 709, SEQ ID NO: 710, SEQ ID NO: 711, SEQ ID NO: 712, SEQ
ID NO: 713, SEQ ID NO: 714, SEQ ID NO: 715, SEQ ID NO: 716, SEQ ID NO: 717, SEQ ID NO: 718, SEQ ID
NO: 719, SEQ ID NO: 720|CD18, ITGB2, LAD, LCAMB, LFA-1, MF17|34, 129, 141, 186, 405, 408, 766|
SEQ ID NO: 54|10|SEQ ID NO: 721, SEQ ID NO: 722, SEQ ID NO: 723, SEQ ID NO: 724, SEQ ID NO: 725, SEQ
ID NO: 726, SEQ ID NO: 727, SEQ ID NO: 728, SEQ ID NO: 729, SEQ ID NO: 730, SEQ ID NO: 731, SEQ ID
NO: 732, SEQ ID NO: 733, SEQ ID NO: 734, SEQ ID NO: 735, SEQ ID NO: 736, SEQ ID NO: 737, SEQ ID
NO: 738, SEQ ID NO: 739, SEQ ID NO: 740, SEQ ID NO: 741, SEQ ID NO: 742, SEQ ID NO: 743, SEQ ID
NO: 744, SEQ ID NO: 745, SEQ ID NO: 746, SEQ ID NO: 747, SEQ ID NO: 748, SEQ ID NO: 749, SEQ ID
NO: 750, SEQ ID NO: 751, SEQ ID NO: 752, SEQ ID NO: 753, SEQ ID NO: 754|BB2, CD54, ICAM1|129, 154, 186,
187, 219, 231, 331, 385, 717, 778|
SEQ ID NO: 55|10|SEQ ID NO: 755, SEQ ID NO: 756, SEQ ID NO: 757, SEQ ID NO: 758, SEQ ID NO: 759, SEQ
ID NO: 760|DUTT1, ROB01|31, 106, 107, 186, 408, 798|
SEQ ID NO: 56|10|SEQ ID NO: 761, SEQ ID NO: 762, SEQ ID NO: 763, SEQ ID NO: 764, SEQ ID NO: 765, SEQ
ID NO: 766, SEQ ID NO: 767, SEQ ID NO: 768, SEQ ID NO: 769, SEQ ID NO: 770, SEQ ID NO: 771, SEQ ID
NO: 772, SEQ ID NO: 773, SEQ ID NO: 774, SEQ ID NO: 775, SEQ ID NO: 776, SEQ ID NO: 777, SEQ ID
NO: 778, SEQ ID NO: 779, SEQ ID NO: 780|VRL, VRL-1|22, 29, 31, 741, 742, 743, 744, 745|
SEQ ID NO: 57|11, 12|SEQ ID NO: 781, SEQ ID NO: 782, SEQ ID NO: 783, SEQ ID NO: 784, SEQ ID NO: 785,
SEQ ID NO: 786, SEQ ID NO: 787, SEQ ID NO: 788, SEQ ID NO: 789, SEQ ID NO: 790, SEQ ID NO: 791|CD97,
TM7LN1|31, 34, 120, 127, 129, 154, 161, 186, 201, 298, 359, 379, 386, 389, 778|
SEQ ID NO: 58|11, 12|SEQ ID NO: 792, SEQ ID NO: 793, SEQ ID NO: 794, SEQ ID NO: 795, SEQ ID NO: 796,
SEQ ID NO: 797, SEQ ID NO: 798, SEQ ID NO: 799, SEQ ID NO: 800, SEQ ID NO: 801, SEQ ID NO: 802, SEQ
ID NO: 803, SEQ ID NO: 804, SEQ ID NO: 805, SEQ ID NO: 806, SEQ ID NO: 807, SEQ ID NO: 808, SEQ ID
NO: 809|FH, FHC, LDLR|31, 76, 89, 154, 219, 331, 464, 510, 778, 825, 826, 827, 828|
SEQ ID NO: 59|11, 12|SEQ ID NO: 792, SEQ ID NO: 793, SEQ ID NO: 794, SEQ ID NO: 795, SEQ ID NO: 796,
SEQ ID NO: 797, SEQ ID NO: 798, SEQ ID NO: 799, SEQ ID NO: 800, SEQ ID NO: 801, SEQ ID NO: 802, SEQ
ID NO: 803, SEQ ID NO: 804, SEQ ID NO: 805, SEQ ID NO: 806, SEQ ID NO: 807, SEQ ID NO: 808, SEQ ID
NO: 809|FH, FHC, LDLR|31, 76, 89, 154, 219, 331, 464, 510, 778, 825, 826, 827, 828|
SEQ ID NO: 60|11, 12|SEQ ID NO: 810, SEQ ID NO: 811, SEQ ID NO: 812, SEQ ID NO: 813, SEQ ID NO: 814|
GP250, LR11, LRP9, SORL1, SORLA|31, 331, 778, 838, 839|
SEQ ID NO: 61|11, 12|SEQ ID NO: 682, SEQ ID NO: 683, SEQ ID NO: 684, SEQ ID NO: 685, SEQ ID NO: 686,
SEQ ID NO: 687, SEQ ID NO: 688|CD71, TFR, TFRC|31, 331, 590, 720, 721, 722|
SEQ ID NO: 62|11, 12|SEQ ID NO: 682, SEQ ID NO: 683, SEQ ID NO: 684, SEQ ID NO: 685, SEQ ID NO: 686,
SEQ ID NO: 687, SEQ ID NO: 688|CD71, TFR, TFRC|31, 331, 590, 720, 721, 722|
SEQ ID NO: 63|11, 12|SEQ ID NO: 682, SEQ ID NO: 683, SEQ ID NO: 684, SEQ ID NO: 685, SEQ ID NO: 686,
SEQ ID NO: 687, SEQ ID NO: 688|CD71, TFR, TFRC|31, 331, 590, 720, 721, 722|
SEQ ID NO: 64|11, 12|SEQ ID NO: 682, SEQ ID NO: 683, SEQ ID NO: 684, SEQ ID NO: 685, SEQ ID NO: 686,
SEQ ID NO: 687, SEQ ID NO: 688|CD71, TFR, TFRC|31, 331, 590, 720, 721, 722|
SEQ ID NO: 65|5|SEQ ID NO: 815, SEQ ID NO: 816, SEQ ID NO: 817, SEQ ID NO: 818, SEQ ID NO: 819, SEQ
ID NO: 820, SEQ ID NO: 821, SEQ ID NO: 822, SEQ ID NO: 823, SEQ ID NO: 824, SEQ ID NO: 825, SEQ ID
NO: 826, SEQ ID NO: 827, SEQ ID NO: 828, SEQ ID NO: 829, SEQ ID NO: 830, SEQ ID NO: 831, SEQ ID
NO: 832, SEQ ID NO: 833, SEQ ID NO: 834|BST2|31, 57, 58, 291, 389, 789|
SEQ ID NO: 66|5|SEQ ID NO: 835, SEQ ID NO: 836, SEQ ID NO: 837, SEQ ID NO: 838, SEQ ID NO: 839, SEQ
ID NO: 840, SEQ ID NO: 841, SEQ ID NO: 842, SEQ ID NO: 843, SEQ ID NO: 844, SEQ ID NO: 845|CD30,
D1S166E, KI-1, TNFRSF8|34, 55, 89, 129, 778|
SEQ ID NO: 67|5|SEQ ID NO: 846, SEQ ID NO: 847, SEQ ID NO: 848, SEQ ID NO: 849, SEQ ID NO: 850, SEQ
ID NO: 851, SEQ ID NO: 852, SEQ ID NO: 853, SEQ ID NO: 854, SEQ ID NO: 855, SEQ ID NO: 856, SEQ ID
NO: 857, SEQ ID NO: 858, SEQ ID NO: 859, SEQ ID NO: 860, SEQ ID NO: 861|IFNGR, IFNGR1|31, 34, 89, 127,
129, 154, 229, 230, 843, 844|
SEQ ID NO: 68|5|SEQ ID NO: 862, SEQ ID NO: 863, SEQ ID NO: 864, SEQ ID NO: 865, SEQ ID NO: 866, SEQ
ID NO: 867, SEQ ID NO: 868, SEQ ID NO: 869, SEQ ID NO: 870, SEQ ID NO: 871, SEQ ID NO: 872, SEQ ID
NO: 873, SEQ ID NO: 874|KIAA0343, NRCAM|2, 31, 107, 186, 187|
SEQ ID NO: 69|5|SEQ ID NO: 875, SEQ ID NO: 876, SEQ ID NO: 877, SEQ ID NO: 878, SEQ ID NO: 879, SEQ
ID NO: 880, SEQ ID NO: 881, SEQ ID NO: 882, SEQ ID NO: 883, SEQ ID NO: 884|CD122, IL2RB|31, 34, 89,
127, 129, 154, 332, 402|
SEQ ID NO: 70|5|SEQ ID NO: 885, SEQ ID NO: 886, SEQ ID NO: 887, SEQ ID NO: 888, SEQ ID NO: 889, SEQ
ID NO: 890, SEQ ID NO: 891, SEQ ID NO: 892, SEQ ID NO: 893, SEQ ID NO: 894, SEQ ID NO: 895, SEQ ID
NO: 896, SEQ ID NO: 897, SEQ ID NO: 898, SEQ ID NO: 899, SEQ ID NO: 900, SEQ ID NO: 901, SEQ ID
NO: 902, SEQ ID NO: 903, SEQ ID NO: 904|CD23, CD23A, FCE2, FCER2, SST|28, 31, 33, 34, 55, 56, 102, 120,
129, 179, 253, 359, 386, 389, 438, 765, 848, 849, 850, 851|
SEQ ID NO: 71|5|SEQ ID NO: 885, SEQ ID NO: 886, SEQ ID NO: 887, SEQ ID NO: 888, SEQ ID NO: 889, SEQ
ID NO: 890, SEQ ID NO: 891, SEQ ID NO: 892, SEQ ID NO: 893, SEQ ID NO: 894, SEQ ID NO: 895, SEQ ID
NO: 896, SEQ ID NO: 897, SEQ ID NO: 898, SEQ ID NO: 899, SEQ ID NO: 900, SEQ ID NO: 901, SEQ ID
NO: 902, SEQ ID NO: 903, SEQ ID NO: 904|CD23, CD23A, FCE2, FCER2, SST|28, 31, 33, 34, 55, 56, 102, 120,
129, 179, 253, 359, 386, 389, 438, 765, 848, 849, 850, 851|
SEQ ID NO: 72|5|SEQ ID NO: 885, SEQ ID NO: 886, SEQ ID NO: 887, SEQ ID NO: 888, SEQ ID NO: 889, SEQ
ID NO: 890, SEQ ID NO: 891, SEQ ID NO: 892, SEQ ID WO: 893, SEQ ID NO: 894, SEQ ID NO: 895, SEQ ID
NO: 896, SEQ ID NO: 897, SEQ ID NO: 898, SEQ ID NO: 899, SEQ ID NO: 900, SEQ ID NO: 901, SEQ ID
NO: 902, SEQ ID NO: 903, SEQ ID NO: 904|CD23, CD23A, FCE2, FCER2, SST|28, 31, 33, 34, 55, 56, 102, 120,
129, 179, 253, 359, 386, 389, 438, 765, 848, 849, 850, 851|
SEQ ID NO: 73|5|SEQ ID NO: 885, SEQ ID NO: 886, SEQ ID NO: 887, SEQ ID NO: 888, SEQ ID NO: 889, SEQ
ID NO: 890, SEQ ID NO: 891, SEQ ID NO: 892, SEQ ID NO: 893, SEQ ID NO: 894, SEQ ID NO: 895, SEQ ID
NO: 896, SEQ ID NO: 897, SEQ ID NO: 898, SEQ ID NO: 899, SEQ ID NO: 900, SEQ ID NO: 901, SEQ ID
NO: 902, SEQ ID NO: 903, SEQ ID NO: 904|CD23, CD23A, FCE2, FCER2, SST|28, 31, 33, 34, 55, 56, 102, 120,
129, 179, 253, 359, 386, 389, 438, 765, 848, 849, 850, 851|
SEQ ID NO: 74|5|SEQ ID NO: 885, SEQ ID NO: 886, SEQ ID NO: 887, SEQ ID NO: 888, SEQ ID NO: 889, SEQ
ID NO: 890, SEQ ID NO: 891, SEQ ID NO: 892, SEQ ID NO: 893, SEQ ID NO: 894, SEQ ID NO: 895, SEQ ID
NO: 896, SEQ ID NO: 897, SEQ ID NO: 898, SEQ ID NO: 899, SEQ ID NO: 900, SEQ ID NO: 901, SEQ ID
NO: 902, SEQ ID NO: 903, SEQ ID NO: 904|CD23, CD23A, FCE2, FCER2, SST|28, 31, 33, 34, 55, 56, 102, 120,
129, 179, 253, 359, 386, 389, 438, 765, 848, 849, 850, 851|
SEQ ID NO: 75|5|SEQ ID NO: 905, SEQ ID NO: 906, SEQ ID NO: 907, SEQ ID NO: 908, SEQ ID NO: 909, SEQ
ID NO: 910|ADAM9, KIAA0021, MCMP, MDC9, MLTNG|31, 47, 51, 212, 640, 765|
SEQ ID NO: 76|5|SEQ ID NO: 911, SEQ ID NO: 912, SEQ ID NO: 913, SEQ ID NO: 914, SEQ ID NO: 915, SEQ
ID NO: 916, SEQ ID NO: 917, SEQ ID NO: 918, SEQ ID NO: 919, SEQ ID NO: 920|HPTPE, PTPE, PTPRE|31, 34,
47, 67, 68, 468, 858|
SEQ ID NO: 77|5|SEQ ID NO: 682, SEQ ID NO: 683, SEQ ID NO: 684, SEQ ID NO: 685, SEQ ID NO: 686, SEQ
ID NO: 687, SEQ ID NO: 688|CD71, TFR, TFRC|31, 331, 590, 720, 721, 722|
SEQ ID NO: 78|5|SEQ ID NO: 682, SEQ ID NO: 683, SEQ ID NO: 684, SEQ ID NO: 685, SEQ ID NO: 921, SEQ
ID NO: 686, SEQ ID NO: 687, SEQ ID NO: 688|CD71, TFR, TFRC|31, 331, 590, 720, 721, 722|
SEQ ID NO: 79|5|SEQ ID NO: 682, SEQ ID NO: 683, SEQ ID NO: 684, SEQ ID NO: 685, SEQ ID NO: 686, SEQ
ID NO: 687, SEQ ID NO: 688|CD71, TFR, TFRC|31, 331, 590, 720, 721, 722|
SEQ ID NO: 80|5|SEQ ID NO: 682, SEQ ID NO: 683, SEQ ID NO: 684, SEQ ID NO: 685, SEQ ID NO: 686, SEQ
ID NO: 687, SEQ ID NO: 688|CD71, TFR, TFRC|31, 331, 590, 720, 721, 722|
SEQ ID NO: 81|5|SEQ ID NO: 682, SEQ ID NO: 683, SEQ ID NO: 684, SEQ ID NO: 685, SEQ ID NO: 686, SEQ
ID NO: 687, SEQ ID NO: 688|CD71, TFR, TFRC|31, 331, 590, 720, 721, 722|
SEQ ID NO: 82|5|SEQ ID NO: 682, SEQ ID NO: 683, SEQ ID NO: 684, SEQ ID NO: 685, SEQ ID NO: 922, SEQ
ID NO: 686, SEQ ID NO: 687, SEQ ID NO: 688|CD71, TFR, TFRC|31, 331, 590, 720, 721, 722|
SEQ ID NO: 83|5|SEQ ID NO: 682, SEQ ID NO: 683, SEQ ID NO: 684, SEQ ID NO: 685, SEQ ID NO: 686, SEQ
ID NO: 687, SEQ ID NO: 688|CD71, TFR, TFRC|31, 331, 590, 720, 721, 722|
SEQ ID NO: 84|5|SEQ ID NO: 682, SEQ ID NO: 683, SEQ ID NO: 684, SEQ ID NO: 685, SEQ ID NO: 686, SEQ
ID NO: 687, SEQ ID NO: 688|CD71, TFR, TFRC|31, 331, 590, 720, 721, 722|
SEQ ID NO: 85|1, 13|SEQ ID NO: 923, SEQ ID NO: 924, SEQ ID NO: 925, SEQ ID NO: 926, SEQ ID NO: 927,
SEQ ID NO: 928|PRKAG1|12, 87, 89, 565, 869|
SEQ ID NO: 86|4|CRL1, TCCR, WSX-1, WSX1|31, 34, 127, 129, 386, 778|
SEQ ID NO: 87|1|SEQ ID NO: 256, SEQ ID NO: 258, SEQ ID NO: 262, SEQ ID NO: 264|CD27L, CD27LG, CD70,
KI-24 ANTIGEN, TNFSF7|31, 56, 57, 89, 120, 179, 387, 388, 389|
SEQ ID NO: 88|18|SEQ ID NO: 929, SEQ ID NO: 930, SEQ ID NO: 931, SEQ ID NO: 932, SEQ ID NO: 933, SEQ
ID NO: 934, SEQ ID NO: 935, SEQ ID NO: 936, SEQ ID NO: 937, SEQ ID NO: 938, SEQ ID NO: 939|CD49F,
ITGA6|34, 200, 224, 405, 408, 923|
SEQ ID NO: 89|18|SEQ ID NO: 929, SEQ ID NO: 930, SEQ ID NO: 931, SEQ ID NO: 933, SEQ ID NO: 935, SEQ
ID NO: 936, SEQ ID NO: 937, SEQ ID NO: 938, SEQ ID NO: 939|CD49F, ITGA6|34, 200, 224, 405, 408, 923|
SEQ ID NO: 90|18|SEQ ID NO: 940, SEQ ID NO: 941, SEQ ID NO: 942, SEQ ID NO: 943, SEQ ID NO: 944, SEQ
ID NO: 945, SEQ ID NO: 946, SEQ ID NO: 947, SEQ ID NO: 948, SEQ ID NO: 949, SEQ ID NO: 950, SEQ ID
NO: 951, SEQ ID NO: 952, SEQ ID NO: 953, SEQ ID NO: 954, SEQ ID NO: 955, SEQ ID NO: 956, SEQ ID
NO: 957, SEQ ID NO: 958, SEQ ID NO: 959, SEQ ID NO: 960, SEQ ID NO: 961, SEQ ID NO: 962, SEQ ID
NO: 963, SEQ ID NO: 964, SEQ ID NO: 965, SEQ ID NO: 966, SEQ ID NO: 967, SEQ ID NO: 968, SEQ ID
NO: 969, SEQ ID NO: 970, SEQ ID NO: 971, SEQ ID NO: 972, SEQ ID NO: 973, SEQ ID NO: 974, SEQ ID
NO: 975, SEQ ID NO: 976, SEQ ID NO: 977|CALM, CLTH, DJ167A19.1, FLT4, KIAA0656, PCL, PICALM, SNAP91,
VEGFR3|12, 31, 34, 135, 332, 411, 461, 639, 815|
SEQ ID NO: 91|19|SEQ ID NO: 978, SEQ ID NO: 979, SEQ ID NO: 980, SEQ ID NO: 981, SEQ ID NO: 982, SEQ
ID NO: 983, SEQ ID NO: 984, SEQ ID NO: 985, SEQ ID NO: 986, SEQ ID NO: 987|CD222, CIMPR, IGF2R, M6P-R,
MPRI|2, 31, 34, 89, 219, 331, 385, 839, 932|
SEQ ID NO: 92|19|SEQ ID NO: 846, SEQ ID NO: 847, SEQ ID NO: 848, SEQ ID NO: 849, SEQ ID NO: 850, SEQ
ID NO: 851, SEQ ID NO: 852, SEQ ID NO: 853, SEQ ID NO: 854, SEQ ID NO: 855, SEQ ID NO: 856, SEQ ID
NO: 857, SEQ ID NO: 858, SEQ ID NO: 859, SEQ ID NO: 860, SEQ ID NO: 861|IFNGR, IFNGR1|31, 34, 89, 127,
129, 154, 229, 230, 843, 844|
SEQ ID NO: 93|20|SEQ ID NO: 988, SEQ ID NO: 989, SEQ ID NO: 990, SEQ ID NO: 991, SEQ ID NO: 992, SEQ
ID NO: 993, SEQ ID NO: 994, SEQ ID NO: 995, SEQ ID NO: 996, SEQ ID NO: 997, SEQ ID NO: 998, SEQ ID
NO: 999, SEQ ID NO: 1000, SEQ ID NO: 1001, SEQ ID NO: 1002, SEQ ID NO: 1003, SEQ ID NO: 1004, SEQ ID
NO: 1005|CALLA, CD10, MME|31, 36, 47, 49, 51, 120, 154, 282, 389, 640|
SEQ ID NO: 94|20|SEQ ID NO: 792, SEQ ID NO: 793, SEQ ID NO: 794, SEQ ID NO: 1006, SEQ ID NO: 795,
SEQ ID NO: 796, SEQ ID NO: 797, SEQ ID NO: 798, SEQ ID NO: 799, SEQ ID NO: 800, SEQ ID NO: 801, SEQ
ID NO: 802, SEQ ID NO: 803, SEQ ID NO: 1007, SEQ ID NO: 804, SEQ ID NO: 805, SEQ ID NO: 806, SEQ ID
NO: 807, SEQ ID NO: 808, SEQ ID NO: 809|FH, FHC, LDLR|31, 76, 89, 154, 219, 331, 464, 510, 778, 825, 826,
827, 828|
SEQ ID NO: 95|22|SEQ ID NO: 1008, SEQ ID NO: 1009, SEQ ID NO: 1010, SEQ ID NO: 1011, SEQ ID
NO: 1012, SEQ ID NO: 1013, SEQ ID NO: 1014, SEQ ID NO: 1015, SEQ ID NO: 1016, SEQ ID NO: 1017, SEQ ID
NO: 1018, SEQ ID NO: 1019, SEQ ID NO: 1020, SEQ ID NO: 1021, SEQ ID NO: 1022, SEQ ID NO: 1023, SEQ ID
NO: 1024, SEQ ID NO: 1025, SEQ ID NO: 1026, SEQ ID NO: 1027, SEQ ID NO: 1028, SEQ ID NO: 1029, SEQ ID
NO: 1030, SEQ ID NO: 1031, SEQ ID NO: 1032, SEQ ID NO: 1033, SEQ ID NO: 1034, SEQ ID NO: 1035, SEQ ID
NO: 1036, SEQ ID NO: 1037, SEQ ID NO: 1038, SEQ ID NO: 1039, SEQ ID NO: 1040|ALK, B23, NPM, NPM1|12,
31, 34, 148, 188, 553|
SEQ ID NO: 96|22|SEQ ID NO: 595, SEQ ID NO: 596, SEQ ID NO: 1041, SEQ ID NO: 597, SEQ ID NO: 598,
SEQ ID NO: 599, SEQ ID NO: 600, SEQ ID NO: 601, SEQ ID NO: 602, SEQ ID NO: 1042, SEQ ID NO: 1043, SEQ
ID NO: 604, SEQ ID NO: 608, SEQ ID NO: 609, SEQ ID NO: 610, SEQ ID NO: 1044|EN01, EN01L1, MBP-1, MPB1,
NNE, PPH|3, 7, 9, 10, 18, 23, 24, 25, 26|
SEQ ID NO: 97|29|SEQ ID NO: 1045, SEQ ID NO: 1046, SEQ ID NO: 1047, SEQ ID NO: 1048, SEQ ID
NO: 1049, SEQ ID NO: 1050, SEQ ID NO: 1051, SEQ ID NO: 1052, SEQ ID NO: 1053, SEQ ID NO: 1054|NDUFS2|
44, 110, 147, 647, 700, 952|
SEQ ID NO: 98|7, 24|SEQ ID NO: 1055, SEQ ID NO: 1056, SEQ ID NO: 1057, SEQ ID NO: 1058, SEQ ID
NO: 1059, SEQ ID NO: 1060, SEQ ID NO: 1061, SEQ ID NO: 1062, SEQ ID NO: 1063|PLXN#, PLXN-C1, PLXNC1,
VESPR|129, 154, 186, 209, 219, 231, 331, 387|
SEQ ID NO: 99|7|SEQ ID NO: 486, SEQ ID NO: 487, SEQ ID NO: 488, SEQ ID NO: 489, SEQ ID NO: 490, SEQ
ID NO: 1064, SEQ ID NO: 491, SEQ ID NO: 494, SEQ ID NO: 495, SEQ ID NO: 496, SEQ ID NO: 497|PCNA|3, 7,
8, 56, 57, 60, 61, 62, 63, 64, 65, 66|
SEQ ID NO: 100|7|SEQ ID NO: 1065, SEQ ID NO: 1066, SEQ ID NO: 1067, SEQ ID NO: 1068, SEQ ID
NO: 1069, SEQ ID NO: 1070, SEQ ID NO: 1071, SEQ ID NO: 1072, SEQ ID NO: 1073, SEQ ID NO: 1074, SEQ ID
NO: 1075, SEQ ID NO: 1076, SEQ ID NO: 1077|CAS, CSE1, CSE1L|8, 56, 57, 67, 179, 219, 249, 250, 388, 499,
956|
SEQ ID NO: 101|7|SEQ ID NO: 1078, SEQ ID NO: 1079, SEQ ID NO: 1080, SEQ ID NO: 1081, SEQ ID
NO: 1082, SEQ ID NO: 1083, SEQ ID NO: 1084, SEQ ID NO: 1085, SEQ ID NO: 1086, SEQ ID NO: 1087, SEQ ID
NO: 1088, SEQ ID NO: 1089, SEQ ID NO: 1090, SEQ ID NO: 1091, SEQ ID NO: 1092, SEQ ID NO: 1093, SEQ ID
NO: 1094, SEQ ID NO: 1095, SEQ ID NO: 1096, SEQ ID NO: 1097, SEQ ID NO: 1098, SEQ ID NO: 1099, SEQ ID
NO: 1100, SEQ ID NO: 1101, SEQ ID NO: 1102, SEQ ID NO: 1103, SEQ ID NO: 1104, SEQ ID NO: 1105, SEQ ID
NO: 1106, SEQ ID NO: 1107, SEQ ID NO: 1108, SEQ ID NO: 1109, SEQ ID NO: 1110, SEQ ID NO: 1111, SEQ ID
NO: 1112, SEQ ID NO: 1113, SEQ ID NO: 1114, SEQ ID NO: 1115, SEQ ID NO: 1116, SEQ ID NO: 1117, SEQ ID
NO: 1118, SEQ ID NO: 1119, SEQ ID NO: 1120, SEQ ID NO: 1121, SEQ ID NO: 1122|MAGE3, MAGE9, MAGEA3,
MAGEA9|297|
SEQ ID NO: 102|7, 24, 29|SEQ ID NO: 1123, SEQ ID NO: 1124, SEQ ID NO: 1125, SEQ ID NO: 1126, SEQ ID
NO: 1127, SEQ ID NO: 1128||ILF2, NF45|7, 8, 10, 54|
SEQ ID NO: 103|7, 13, 14, 28|SEQ ID NO: 1129, SEQ ID NO: 1130, SEQ ID NO: 1131, SEQ ID NO: 1132, SEQ
ID NO: 1133|DXS423E, KIAA0178, SB1.8, SMC1, SMC1 (ALPHA), SMC1L1, SMCB|8, 13, 35, 84, 363, 365, 601, 960|
SEQ ID NO: 104|7, 13, 14|SEQ ID NO: 1134, SEQ ID NO: 1135, SEQ ID NO: 1136, SEQ ID NO: 1137|TIL3,
TLR5|31, 34, 89, 127, 129, 385, 961|
SEQ ID NO: 105|7, 14|SEQ ID NO: 1138, SEQ ID NO: 1139, SEQ ID NO: 1140, SEQ ID NO: 1141, SEQ ID
NO: 1142, SEQ ID NO: 1143, SEQ ID NO: 1144, SEQ ID NO: 1145, SEQ ID NO: 1146|DNAJA1, DNAJA1-PENDING,
HSJ2, HSPF4|218, 257, 384, 390, 477|
SEQ ID NO: 106|7, 14|SEQ ID NO: 1147, SEQ ID NO: 1148, SEQ ID NO: 1149, SEQ ID NO: 1150|NE01, NGN|2,
31, 58, 107, 120, 154, 161, 186, 187, 201, 209, 291, 389|
SEQ ID NO: 107|13|SEQ ID NO: 1151, SEQ ID NO: 1152, SEQ ID NO: 1153, SEQ ID NO: 1154, SEQ ID
NO: 1155, SEQ ID NO: 1156, SEQ ID NO: 1157, SEQ ID NO: 1158, SEQ ID NO: 1159, SEQ ID NO: 1160, SEQ ID
NO: 1161, SEQ ID NO: 1162, SEQ ID NO: 1163, SEQ ID NO: 1164, SEQ ID NO: 1165, SEQ ID NO: 1166, SEQ ID
NO: 1167, SEQ ID NO: 1168, SEQ ID NO: 1169, SEQ ID NO: 1170, SEQ ID NO: 1171, SEQ ID NO: 1172, SEQ ID
NO: 1173, SEQ ID NO: 1174, SEQ ID NO: 1175, SEQ ID NO: 1176, SEQ ID NO: 1177, SEQ ID NO: 1178, SEQ ID
NO: 1179, SEQ ID NO: 1180, SEQ ID NO: 1181, SEQ ID NO: 1182, SEQ ID NO: 1183, SEQ ID NO: 1184, SEQ ID
NO: 1185, SEQ ID NO: 1186, SEQ ID NO: 1187, SEQ ID NO: 1188, SEQ ID NO: 1189, SEQ ID NO: 1190, SEQ ID
NO: 1191, SEQ ID NO: 1192, SEQ ID NO: 1193, SEQ ID NO: 1194, SEQ ID NO: 1195, SEQ ID NO: 1196, SEQ ID
NO: 1197, SEQ ID NO: 1198, SEQ ID NO: 1199, SEQ ID NO: 1200, SEQ ID NO: 1201, SEQ ID NO: 1202, SEQ ID
NO: 1203, SEQ ID NO: 1204, SEQ ID NO: 1205, SEQ ID NO: 1206, SEQ ID NO: 1207, SEQ ID NO: 1208, SEQ ID
NO: 1209, SEQ ID NO: 1210, SEQ ID NO: 1211, SEQ ID NO: 1212, SEQ ID NO: 1213, SEQ ID NO: 1214, SEQ ID
NO: 1215, SEQ ID NO: 1216, SEQ ID NO: 1217, SEQ ID NO: 1218, SEQ ID NO: 1219, SEQ ID NO: 1220, SEQ ID
NO: 1221, SEQ ID NO: 1222, SEQ ID NO: 1223, SEQ ID NO: 1224, SEQ ID NO: 1225, SEQ ID NO: 1226, SEQ ID
NO: 1227, SEQ ID NO: 1228, SEQ ID NO: 1229, SEQ ID NO: 1230, SEQ ID NO: 1231, SEQ ID NO: 1232, SEQ ID
NO: 1233, SEQ ID NO: 1234, SEQ ID NO: 1235, SEQ ID NO: 1236, SEQ ID NO: 1237, SEQ ID NO: 1238, SEQ ID
NO: 1239, SEQ ID NO: 1240, SEQ ID NO: 1241|BP1|31, 127, 129, 969|
SEQ ID NO: 108|13, 28|SEQ ID NO: 1242, SEQ ID NO: 1243, SEQ ID NO: 1244, SEQ ID NO: 1245|CD66C,
CEACAM6, NCA|31, 34, 89, 120, 389|
SEQ ID NO: 109|13, 14|SEQ ID NO: 1246, SEQ ID NO: 1247, SEQ ID NO: 1248, SEQ ID NO: 1249, SEQ ID
NO: 1250, SEQ ID NO: 1251, SEQ ID NO: 1252, SEQ ID NO: 1253, SEQ ID NO: 1254, SEQ ID NO: 1255, SEQ ID
NO: 1256, SEQ ID NO: 1257|ANM1, HCP1, HRMT1L2, IR1B4, PRMT1|12, 67, 129, 192, 386, 537, 973, 974|
SEQ ID NO: 110|13|SEQ ID NO: 1258, SEQ ID NO: 1259, SEQ ID NO: 1260, SEQ ID NO: 1261, SEQ ID
NO: 1262, SEQ ID NO: 1263, SEQ ID NO: 1264, SEQ ID NO: 1265, SEQ ID NO: 1266, SEQ ID NO: 1267|PHB|2|
SEQ ID NO: 111|8, 15|SEQ ID NO: 1268, SEQ ID NO: 1269, SEQ ID NO: 1270, SEQ ID NO: 1271, SEQ ID
NO: 1272, SEQ ID NO: 1273, SEQ ID NO: 1274, SEQ ID NO: 1275, SEQ ID NO: 1276, SEQ ID NO: 1277, SEQ ID
NO: 1278, SEQ ID NO: 1279, SEQ ID NO: 1280, SEQ ID NO: 1281, SEQ ID NO: 1282, SEQ ID NO: 1283, SEQ ID
NO: 1284, SEQ ID NO: 1285, SEQ ID NO: 1286, SEQ ID NO: 1287, SEQ ID NO: 1288, SEQ ID NO: 1289, SEQ ID
NO: 1290, SEQ ID NO: 1291, SEQ ID NO: 1292, SEQ ID NO: 1293, SEQ ID NO: 1294, SEQ ID NO: 1295, SEQ ID
NO: 1296, SEQ ID NO: 1297, SEQ ID NO: 1298, SEQ ID NO: 1299, SEQ ID NO: 1300, SEQ ID NO: 1301, SEQ ID
NO: 1302, SEQ ID NO: 1303, SEQ ID NO: 1304, SEQ ID NO: 1305, SEQ ID NO: 1306, SEQ ID NO: 1307, SEQ ID
NO: 1308, SEQ ID NO: 1309, SEQ ID NO: 1310, SEQ ID NO: 1311, SEQ ID NO: 1312, SEQ ID NO: 1313, SEQ ID
NO: 1314, SEQ ID NO: 1315, SEQ ID NO: 1316, SEQ ID NO: 1317, SEQ ID NO: 1318, SEQ ID NO: 1319|CD44,
CD44R, IN, MC56, MDU2, MDU3, MIC4|
SEQ ID NO: 112|8, 15|SEQ ID NO: 1320, SEQ ID NO: 1321, SEQ ID NO: 1322, SEQ ID NO: 1323, SEQ ID
NO: 1324, SEQ ID NO: 1325, SEQ ID NO: 1326, SEQ ID NO: 1327, SEQ ID NO: 1328, SEQ ID NO: 1329, SEQ ID
NO: 1330, SEQ ID NO: 1331, SEQ ID NO: 1332, SEQ ID NO: 1333, SEQ ID NO: 1334, SEQ ID NO: 1335, SEQ ID
NO: 1336, SEQ ID NO: 1337, SEQ ID NO: 1338, SEQ ID NO: 1339|37LRP, LAMBR, LAMR1, LRP, P40, RPSA|31, 34,
36, 72, 162, 186, 352, 386, 395, 396, 397|
SEQ ID NO: 113|15|SEQ ID NO: 1340, SEQ ID NO: 1341, SEQ ID NO: 1342, SEQ ID NO: 1343, SEQ ID
NO: 1344, SEQ ID NO: 1345, SEQ ID NO: 1346, SEQ ID NO: 1347, SEQ ID NO: 1348, SEQ ID NO: 1349, SEQ ID
NO: 1350, SEQ ID NO: 1351, SEQ ID NO: 1352, SEQ ID NO: 1353|GALBP, LGALS2, LGALS3, MAC-2, MAC2|56,
154, 635, 1012|
SEQ ID NO: 114|15|SEQ ID NO: 1340, SEQ ID NO: 1341, SEQ ID NO: 1342, SEQ ID NO: 1343, SEQ ID
NO: 1344, SEQ ID NO: 1345, SEQ ID NO: 1346, SEQ ID NO: 1347, SEQ ID NO: 1348, SEQ ID NO: 1349, SEQ ID
NO: 1351, SEQ ID NO: 1352, SEQ ID NO: 1353|GALBP, LGALS2, LGALS3, MAC-2, MAC2|56, 154, 635, 1012|
SEQ ID NO: 115|13, 14|SEQ ID NO: 1354, SEQ ID NO: 1355, SEQ ID NO: 1356, SEQ ID NO: 1357, SEQ ID
NO: 1358|ECT2|
SEQ ID NO: 116|13|SEQ ID NO: 1359, SEQ ID NO: 1360, SEQ ID NO: 1361, SEQ ID NO: 1362, SEQ ID
NO: 1363, SEQ ID NO: 1364, SEQ ID NO: 1365, SEQ ID NO: 1366|CSNU3, SLC7A9|29, 31, 111, 154, 332, 712,
777, 1035, 1036|
SEQ ID NO: 117|13|SEQ ID NO: 1367, SEQ ID NO: 1368, SEQ ID NO: 1369, SEQ ID NO: 1370|BAF155,
CRACC1, SMARCC1, SRG3|10, 118, 151, 189, 199|
SEQ ID NO: 118|13|SEQ ID NO: 1371, SEQ ID NO: 1372, SEQ ID NO: 1373, SEQ ID NO: 1374, SEQ ID
NO: 1375, SEQ ID NO: 1376, SEQ ID NO: 1377, SEQ ID NO: 1378, SEQ ID NO: 1379, SEQ ID NO: 1380, SEQ ID
NO: 1381, SEQ ID NO: 1382, SEQ ID NO: 1383, SEQ ID NO: 1384, SEQ ID NO: 1385, SEQ ID NO: 1386|PDK1,
PDPK1|12, 87, 104, 105, 133, 512, 1044|
SEQ ID NO: 119|13|SEQ ID NO: 1387, SEQ ID NO: 1388, SEQ ID NO: 1389, SEQ ID NO: 1390, SEQ ID
NO: 1391, SEQ ID NO: 1392, SEQ ID NO: 1393, SEQ ID NO: 1394, SEQ ID NO: 1395, SEQ ID NO: 1396, SEQ ID
NO: 1397, SEQ ID NO: 1398, SEQ ID NO: 1399, SEQ ID NO: 1400|CKS1|57, 62, 137, 529|
SEQ ID NO: 120|13, 24|SEQ ID NO: 1401, SEQ ID NO: 1402, SEQ ID NO: 1403, SEQ ID NO: 1404, SEQ ID
NO: 1405, SEQ ID NO: 1406, SEQ ID NO: 1407, SEQ ID NO: 1408, SEQ ID NO: 1409, SEQ ID NO: 1410, SEQ ID
NO: 1411, SEQ ID NO: 1412, SEQ ID NO: 1413, SEQ ID NO: 1414, SEQ ID NO: 1415, SEQ ID NO: 1416|ISGF-3,
STAT1, STAT91|3, 7, 8, 10, 16, 23, 53, 62, 67, 89, 179, 263, 265, 781, 782, 783, 784, 785, 786|
SEQ ID NO: 121|13|SEQ ID NO: 1417, SEQ ID NO: 1418, SEQ ID NO: 1419, SEQ ID NO: 1420, SEQ ID
NO: 1421, SEQ ID NO: 1422, SEQ ID NO: 1423, SEQ ID NO: 1424, SEQ ID NO: 1425, SEQ ID NO: 1426, SEQ ID
NO: 1427, SEQ ID NO: 1428, SEQ ID NO: 1429, SEQ ID NO: 1430, SEQ ID NO: 1431, SEQ ID NO: 1432, SEQ ID
NO: 1433, SEQ ID NO: 1434, SEQ ID NO: 1435, SEQ ID NO: 1436, SEQ ID NO: 1437, SEQ ID NO: 1438|GPCR,
GPR34|31, 34, 359, 379|
SEQ ID NO: 122|13|SEQ ID NO: 1439, SEQ ID NO: 1440, SEQ ID NO: 1441, SEQ ID NO: 1442, SEQ ID
NO: 1443|HTOLL, TLR4|31, 34, 89, 127, 129, 385, 961|
SEQ ID NO: 123|8, 15|SEQ ID NO: 1321, SEQ ID NO: 1322, SEQ ID NO: 1323, SEQ ID NO: 1324, SEQ ID
NO: 1325, SEQ ID NO: 1326, SEQ ID NO: 1327, SEQ ID NO: 1328, SEQ ID NO: 1329, SEQ ID NO: 1330, SEQ ID
NO: 1331, SEQ ID NO: 1332, SEQ ID NO: 1333, SEQ ID NO: 1444, SEQ ID NO: 1445, SEQ ID NO: 1446, SEQ ID
NO: 1447, SEQ ID NO: 1336, SEQ ID NO: 1337, SEQ ID NO: 1338, SEQ ID NO: 1339|37LRP, LAMBR, LAMR1, LRP,
P40, RPSA|31, 34, 36, 72, 162, 186, 352, 386, 395, 396, 397|
SEQ ID NO: 124|15|SEQ ID NO: 1340, SEQ ID NO: 1341, SEQ ID NO: 1342, SEQ ID NO: 1343, SEQ ID
NO: 1344, SEQ ID NO: 1345, SEQ ID NO: 1346, SEQ ID NO: 1347, SEQ ID NO: 1348, SEQ ID NO: 1350, SEQ ID
NO: 1448, SEQ ID NO: 1351, SEQ ID NO: 1352, SEQ ID NO: 1353|GALBP, LGALS2, LGALS3, MAC-2, MAC2|56,
154, 635, 1012|
SEQ ID NO: 125|15|SEQ ID NO: 1449, SEQ ID NO: 1450, SEQ ID NO: 1451, SEQ ID NO: 1452, SEQ ID
NO: 1453, SEQ ID NO: 1454, SEQ ID NO: 1455, SEQ ID NO: 1456, SEQ ID NO: 1457, SEQ ID NO: 1458, SEQ ID
NO: 1459, SEQ ID NO: 1460, SEQ ID NO: 1461|IA-2, IA2, PTPRN|31, 34, 47, 468, 858|
SEQ ID NO: 126|13|SEQ ID NO: 1242, SEQ ID NO: 1462, SEQ ID NO: 1463, SEQ ID NO: 1464, SEQ ID
NO: 1465, SEQ ID NO: 1466, SEQ ID NO: 1467, SEQ ID NO: 1468, SEQ ID NO: 1243, SEQ ID NO: 1469, SEQ ID
NO: 1470, SEQ ID NO: 1471, SEQ ID NO: 1472, SEQ ID NO: 1473, SEQ ID NO: 1474, SEQ ID NO: 1475, SEQ ID
NO: 1476, SEQ ID NO: 1477, SEQ ID NO: 1478, SEQ ID NO: 1479, SEQ ID NO: 1480, SEQ ID NO: 1481, SEQ ID
NO: 1482, SEQ ID NO: 1244, SEQ ID NO: 1483, SEQ ID NO: 1484, SEQ ID NO: 1485, SEQ ID NO: 1486, SEQ ID
NO: 1487, SEQ ID NO: 1488, SEQ ID NO: 1489, SEQ ID NO: 1490, SEQ ID NO: 1491, SEQ ID NO: 1492, SEQ ID
NO: 1493, SEQ ID NO: 1494, SEQ ID NO: 1495, SEQ ID NO: 1496, SEQ ID NO: 1497, SEQ ID NO: 1498, SEQ ID
NO: 1499, SEQ ID NO: 1500, SEQ ID NO: 1501, SEQ ID NO: 1502, SEQ ID NO: 1503, SEQ ID NO: 1504, SEQ ID
NO: 1505, SEQ ID NO: 1506, SEQ ID NO: 1507, SEQ ID NO: 1245|CD66B, CD66C, CD66E, CD67, CEA, CEACAM5,
CEACAM6, CEACAM8, CGM6, NCA, NCA-95|31, 34, 89, 120, 127, 129, 154, 297, 389, 438|
SEQ ID NO: 127|13|SEQ ID NO: 1462, SEQ ID NO: 1464, SEQ ID NO: 1465, SEQ ID NO: 1466, SEQ ID
NO: 1468, SEQ ID NO: 1472, SEQ ID NO: 1475, SEQ ID NO: 1476, SEQ ID NO: 1478, SEQ ID NO: 1508, SEQ ID
NO: 1479, SEQ ID NO: 1482, SEQ ID NO: 1483, SEQ ID NO: 1485, SEQ ID NO: 1488, SEQ ID NO: 1489, SEQ ID
NO: 1491, SEQ ID NO: 1493, SEQ ID NO: 1509, SEQ ID NO: 1510, SEQ ID NO: 1511, SEQ ID NO: 1496, SEQ ID
NO: 1512, SEQ ID NO: 1513, SEQ ID NO: 1514, SEQ ID NO: 1500, SEQ ID NO: 1503|CD66E, CEA, CEACAM5|31,
297|
SEQ ID NO: 128|25, 26|SEQ ID NO: 682, SEQ ID NO: 683, SEQ ID NO: 684, SEQ ID NO: 685, SEQ ID NO: 686,
SEQ ID NO: 687, SEQ ID NO: 688|CD71, TFR, TFRC|31, 331, 590, 720, 721, 722|
SEQ ID NO: 129|25, 26|SEQ ID NO: 810, SEQ ID NO: 811, SEQ ID NO: 812, SEQ ID NO: 813, SEQ ID NO: 814|
GP250, LR11, LRP9, SORL1, SORLA|31, 331, 778, 838, 839|
SEQ ID NO: 130|25, 26|SEQ ID NO: 1515, SEQ ID NO: 1516, SEQ ID NO: 1517, SEQ ID NO: 1518, SEQ ID
NO: 1519, SEQ ID NO: 1520, SEQ ID NO: 1521, SEQ ID NO: 1522, SEQ ID NO: 1523, SEQ ID NO: 1524, SEQ ID
NO: 1525, SEQ ID NO: 1526, SEQ ID NO: 1527, SEQ ID NO: 1528, SEQ ID NO: 1529, SEQ ID NO: 1530, SEQ ID
NO: 1531, SEQ ID NO: 1532, SEQ ID NO: 1533, SEQ ID NO: 1534, SEQ ID NO: 1535|B1, BP35, CD20, MS4A1,
MS4A1A, MS4A2|22, 31, 57, 129, 359, 789, 1110|
SEQ ID NO: 131|25, 26|SEQ ID NO: 1515, SEQ ID NO: 1516, SEQ ID NO: 1517, SEQ ID NO: 1518, SEQ ID
NO: 1519, SEQ ID NO: 1520, SEQ ID NO: 1521, SEQ ID NO: 1522, SEQ ID NO: 1523, SEQ ID NO: 1524, SEQ ID
NO: 1525, SEQ ID NO: 1526, SEQ ID NO: 1527, SEQ ID NO: 1528, SEQ ID NO: 1529, SEQ ID NO: 1530, SEQ ID
NO: 1531, SEQ ID NO: 1532, SEQ ID NO: 1533, SEQ ID NO: 1534, SEQ ID NO: 1535|B1, BP35, CD20, MS4A1,
MS4A1A, MS4A2|22, 31, 57, 129, 359, 789, 1110|
SEQ ID NO: 132|25, 26|SEQ ID NO: 1515, SEQ ID NO: 1516, SEQ ID NO: 1517, SEQ ID NO: 1518, SEQ ID
NO: 1519, SEQ ID NO: 1520, SEQ ID NO: 1521, SEQ ID NO: 1522, SEQ ID NO: 1523, SEQ ID NO: 1524, SEQ ID
NO: 1525, SEQ ID NO: 1526, SEQ ID NO: 1527, SEQ ID NO: 1528, SEQ ID NO: 1529, SEQ ID NO: 1530, SEQ ID
NO: 1531, SEQ ID NO: 1532, SEQ ID NO: 1533, SEQ ID NO: 1534, SEQ ID NO: 1535|B1, BP35, CD20, MS4A1,
MS4A1A, MS4A2|22, 31, 57, 129, 359, 789, 1110|
SEQ ID NO: 133|25, 26|SEQ ID NO: 682, SEQ ID NO: 683, SEQ ID NO: 684, SEQ ID NO: 685, SEQ ID NO: 686,
SEQ ID NO: 687, SEQ ID NO: 688|CD71, TFR, TFRC|31, 331, 590, 720, 721, 722|
SEQ ID NO: 134|28|SEQ ID NO: 1536, SEQ ID NO: 1537, SEQ ID NO: 1538, SEQ ID NO: 1539, SEQ ID
NO: 1540|WNT5A|36, 68, 89, 91, 120, 345, 387, 389, 438, 1114|
SEQ ID NO: 135|28|SEQ ID NO: 1541, SEQ ID NO: 1542, SEQ ID NO: 1543, SEQ ID NO: 1544|CB2, CNR2|31,
34, 127, 129, 154, 754, 1116, 1117, 1118|
SEQ ID NO: 136|28|SEQ ID NO: 1545, SEQ ID NO: 1546, SEQ ID NO: 1547, SEQ ID NO: 1548, SEQ ID
NO: 1549, SEQ ID NO: 1550, SEQ ID NO: 1551, SEQ ID NO: 1552, SEQ ID NO: 1553, SEQ ID NO: 1554|RRM1|
SEQ ID NO: 137|24|SEQ ID NO: 486, SEQ ID NO: 487, SEQ ID NO: 488, SEQ ID NO: 489, SEQ ID-NO: 490,
SEQ ID NO: 1064, SEQ ID NO: 491, SEQ ID NO: 494, SEQ ID NO: 495, SEQ ID NO: 496, SEQ ID NO: 497|PCNA|
3, 7, 8, 56, 57, 60, 61, 62, 63, 64, 65, 66|
SEQ ID NO: 138|24|SEQ ID NO: 1401, SEQ ID NO: 1402, SEQ ID NO: 1403, SEQ ID NO: 1555, SEQ ID
NO: 1405, SEQ ID NO: 1406, SEQ ID NO: 1407, SEQ ID NO: 1408, SEQ ID NO: 1409, SEQ ID NO: 1410, SEQ ID
NO: 1411, SEQ ID NO: 1412, SEQ ID NO: 1413, SEQ ID NO: 1414, SEQ ID NO: 1415, SEQ ID NO: 1416|ISGF-3,
STAT1, STAT91|3, 7, 8, 10, 16, 23, 53, 62, 67, 89, 179, 263, 265, 781, 782, 783, 784, 785, 786|
SEQ ID NO: 139|24|SEQ ID NO: 1556, SEQ ID NO: 1557, SEQ ID NO: 1558, SEQ ID NO: 1559, SEQ ID
NO: 1560|PCM1|67, 134, 501, 1121|
SEQ ID NO: 140|24|SEQ ID NO: 1561, SEQ ID NO: 1562, SEQ ID NO: 1563, SEQ ID NO: 1564, SEQ ID
NO: 1565, SEQ ID NO: 1566, SEQ ID NO: 1567, SEQ ID NO: 1568, SEQ ID NO: 1569|CADTK, CAK BETA, CAKB,
FAK2, PKB, PTK, PTK2B, PYK2, RAFTK|12, 56, 67, 87, 88, 89, 179, 186, 332, 344, 388, 448, 579, 1122, 1123|
SEQ ID NO: 141|1|SEQ ID NO: 1570, SEQ ID NO: 1571, SEQ ID NO: 1572, SEQ ID NO: 1573, SEQ ID
NO: 1574, SEQ ID NO: 1575, SEQ ID NO: 1576, SEQ ID NO: 1577|TETRAN, TETTRAN|29, 209, 711, 712, 713|
SEQ ID NO: 142|13|SEQ ID NO: 1578, SEQ ID NO: 1579, SEQ ID NO: 1580, SEQ ID NO: 1581, SEQ ID
NO: 1582, SEQ ID NO: 1583, SEQ ID NO: 1584, SEQ ID NO: 1585, SEQ ID NO: 1586, SEQ ID NO: 1587, SEQ ID
NO: 1588, SEQ ID NO: 1589, SEQ ID NO: 1590, SEQ ID NO: 1591, SEQ ID NO: 1592, SEQ ID NO: 1593, SEQ ID
NO: 1594, SEQ ID NO: 1595, SEQ ID NO: 1596, SEQ ID NO: 1597, SEQ ID NO: 1598, SEQ ID NO: 1599, SEQ ID
NO: 1600, SEQ ID NO: 1601, SEQ ID NO: 1602, SEQ ID NO: 1603|MT-SP2, TMPRSS3, TMPRSS4|
SEQ ID NO: 143|15|SEQ ID NO: 929, SEQ ID NO: 930, SEQ ID NO: 931, SEQ ID NO: 932, SEQ ID NO: 933,
SEQ ID NO: 1604, SEQ ID NO: 934, SEQ ID NO: 935, SEQ ID NO: 936, SEQ ID NO: 937, SEQ ID NO: 938, SEQ
ID NO: 939|CD49F, ITGA6|34, 200, 224, 405, 408, 923|
SEQ ID NO: 144|18|SEQ ID NO: 1605, SEQ ID NO: 1606, SEQ ID NO: 1607, SEQ ID NO: 1608, SEQ ID
NO: 1609, SEQ ID NO: 1610, SEQ ID NO: 1611, SEQ ID NO: 1612, SEQ ID NO: 1613, SEQ ID NO: 1614, SEQ ID
NO: 1615, SEQ ID NO: 1616, SEQ ID NO: 1617, SEQ ID NO: 1618, SEQ ID NO: 1619, SEQ ID NO: 1620, SEQ ID
NO: 1621, SEQ ID NO: 1622, SEQ ID NO: 1623, SEQ ID NO: 1624, SEQ ID NO: 1625, SEQ ID NO: 1626, SEQ ID
NO: 1627, SEQ ID NO: 1628, SEQ ID NO: 1629, SEQ ID NO: 1630, SEQ ID NO: 1631, SEQ ID NO: 1632, SEQ ID
NO: 1633, SEQ ID NO: 1634, SEQ ID NO: 1635, SEQ ID NO: 1636, SEQ ID NO: 1637, SEQ ID NO: 1638, SEQ ID
NO: 1639, SEQ ID NO: 1640|B-ALPHA-1, K-ALPHA-1, MGC10851, MGC14580, TUBA3, TUBA6|106, 132, 134, 163,
319, 320, 321|
SEQ ID NO: 145|28|SEQ ID NO: 1641, SEQ ID NO: 1642, SEQ ID NO: 1643, SEQ ID NO: 1644, SEQ ID
NO: 1645, SEQ ID NO: 1646, SEQ ID NO: 1647|CD49C, GAPB3, ITGA3, VL3A|34, 200, 405, 408|
SEQ ID NO: 146|28|SEQ ID NO: 1648, SEQ ID NO: 1649, SEQ ID NO: 1650, SEQ ID NO: 1651, SEQ ID
NO: 1652, SEQ ID NO: 1653, SEQ ID NO: 1654, SEQ ID NO: 1655, SEQ ID NO: 1656, SEQ ID NO: 1657, SEQ ID
NO: 1658, SEQ ID NO: 1659|CMD1A, EMD2, FPL, FPLD, LDP1, LFP, LMN1, LMNA|132, 134, 320, 608, 627, 628|
SEQ ID NO: 147|24|SEQ ID NO: 689, SEQ ID NO: 1660, SEQ ID NO: 690, SEQ ID NO: 691, SEQ ID NO: 692,
SEQ ID NO: 693, SEQ ID NO: 694, SEQ ID NO: 695, SEQ ID NO: 696, SEQ ID NO: 1661|CD49D, ITGA4|31, 34,
186, 408|
SEQ ID NO: 148|24|SEQ ID NO: 1662, SEQ ID NO: 1663, SEQ ID NO: 1664, SEQ ID NO: 1665, SEQ ID
NO: 1666, SEQ ID NO: 1667, SEQ ID NO: 1668, SEQ ID NO: 1669, SEQ ID NO: 1670, SEQ ID NO: 1671, SEQ ID
NO: 1672, SEQ ID NO: 1673|B29, CD79B, IGB|31, 89, 127, 129|
SEQ ID NO: 149|24|SEQ ID NO: 486, SEQ ID NO: 487, SEQ ID NO: 488, SEQ ID NO: 489, SEQ ID NO: 490,
SEQ ID NO: 491, SEQ ID NO: 493, SEQ ID NO: 494, SEQ ID NO: 495, SEQ ID NO: 496, SEQ ID NO: 497|PCNA|
3, 7, 8, 56, 57, 60, 61, 62, 63, 64, 65, 66|
SEQ ID NO: 150|24|SEQ ID NO: 611, SEQ ID NO: 612, SEQ ID NO: 613, SEQ ID NO: 614, SEQ ID NO: 615,
SEQ ID NO: 616, SEQ ID NO: 617, SEQ ID NO: 618|ITGB7|34, 186, 405, 408|
SEQ ID NO: 151|11|SEQ ID NO: 1462, SEQ ID NO: 1464, SEQ ID NO: 1465, SEQ ID NO: 1466, SEQ ID
NO: 1674, SEQ ID NO: 1675, SEQ ID NO: 1676, SEQ ID NO: 1677, SEQ ID NO: 1472, SEQ ID NO: 1678, SEQ ID
NO: 1475, SEQ ID NO: 1679, SEQ ID NO: 1680, SEQ ID NO: 1478, SEQ ID NO: 1482, SEQ ID NO: 1483, SEQ ID
NO: 1681, SEQ ID NO: 1487, SEQ ID NO: 1488, SEQ ID NO: 1489, SEQ ID NO: 1493, SEQ ID NO: 1682, SEQ ID
NO: 1683, SEQ ID NO: 1684, SEQ ID NO: 1685, SEQ ID NO: 1686, SEQ ID NO: 1687, SEQ ID NO: 1688, SEQ ID
NO: 1689, SEQ ID NO: 1690, SEQ ID NO: 1691, SEQ ID NO: 1692, SEQ ID NO: 1693, SEQ ID NO: 1694, SEQ ID
NO: 1695, SEQ ID NO: 1696, SEQ ID NO: 1697, SEQ ID NO: 1500, SEQ ID NO: 1698, SEQ ID NO: 1503, SEQ ID
NO: 1699, SEQ ID NO: 1700, SEQ ID NO: 1701|CD66D, CD66E, CEA, CEACAM3, CEACAM5, CGM1|31, 297|
SEQ ID NO: 152|11|SEQ ID NO: 1462, SEQ ID NO: 1464, SEQ ID NO: 1465, SEQ ID NO: 1466, SEQ ID
NO: 1674, SEQ ID NO: 1675, SEQ ID NO: 1676, SEQ ID NO: 1677, SEQ ID NO: 1472, SEQ ID NO: 1678, SEQ ID
NO: 1475, SEQ ID NO: 1679, SEQ ID NO: 1680, SEQ ID NO: 1478, SEQ ID NO: 1482, SEQ ID NO: 1483, SEQ ID
NO: 1681, SEQ ID NO: 1487, SEQ ID NO: 1488, SEQ ID NO: 1489, SEQ ID NO: 1493, SEQ ID NO: 1682, SEQ ID
NO: 1683, SEQ ID NO: 1684, SEQ ID NO: 1685, SEQ ID NO: 1686, SEQ ID NO: 1687, SEQ ID NO: 1688, SEQ ID
NO: 1689, SEQ ID NO: 1690, SEQ ID NO: 1691, SEQ ID NO: 1692, SEQ ID NO: 1693, SEQ ID NO: 1694, SEQ ID
NO: 1695, SEQ ID NO: 1696, SEQ ID NO: 1697, SEQ ID NO: 1500, SEQ ID NO: 1698, SEQ ID NO: 1503, SEQ ID
NO: 1699, SEQ ID NO: 1700, SEQ ID NO: 1701|CD66D, CD66E, CEA, CEACAM3, CEACAM5, CGM1|31, 297|
SEQ ID NO: 153|11|SEQ ID NO: 1462, SEQ ID NO: 1464, SEQ ID NO: 1465, SEQ ID NO: 1466, SEQ ID
NO: 1674, SEQ ID NO: 1675, SEQ ID NO: 1676, SEQ ID NO: 1677, SEQ ID NO: 1472, SEQ ID NO: 1678, SEQ ID
NO: 1475, SEQ ID NO: 1679, SEQ ID NO: 1680, SEQ ID NO: 1478, SEQ ID NO: 1482, SEQ ID NO: 1483, SEQ ID
NO: 1681, SEQ ID NO: 1487, SEQ ID NO: 1488, SEQ ID NO: 1489, SEQ ID NO: 1493, SEQ ID NO: 1682, SEQ ID
NO: 1683, SEQ ID NO: 1684, SEQ ID NO: 1685, SEQ ID NO: 1686, SEQ ID NO: 1687, SEQ ID NO: 1688, SEQ ID
NO: 1689, SEQ ID NO: 1690, SEQ ID NO: 1691, SEQ ID NO: 1692, SEQ ID NO: 1693, SEQ ID NO: 1694, SEQ ID
NO: 1695, SEQ ID NO: 1696, SEQ ID NO: 1697, SEQ ID NO: 1500, SEQ ID NO: 1698, SEQ ID NO: 1503, SEQ ID
NO: 1699, SEQ ID NO: 1700, SEQ ID NO: 1701|CD66D, CD66E, CEA, CEACAM3, CEACAM5, CGM1|31, 297|
SEQ ID NO: 154|11|SEQ ID NO: 1462, SEQ ID NO: 1464, SEQ ID NO: 1465, SEQ ID NO: 1466, SEQ ID
NO: 1674, SEQ ID NO: 1675, SEQ ID NO: 1676, SEQ ID NO: 1677, SEQ ID NO: 1472, SEQ ID NO: 1678, SEQ ID
NO: 1475, SEQ ID NO: 1679, SEQ ID NO: 1680, SEQ ID NO: 1478, SEQ ID NO: 1482, SEQ ID NO: 1483, SEQ ID
NO: 1681, SEQ ID NO: 1487, SEQ ID NO: 1488, SEQ ID NO: 1489, SEQ ID NO: 1493, SEQ ID NO: 1682, SEQ ID
NO: 1683, SEQ ID NO: 1684, SEQ ID NO: 1685, SEQ ID NO: 1686, SEQ ID NO: 1687, SEQ ID NO: 1688, SEQ ID
NO: 1689, SEQ ID NO: 1690, SEQ ID NO: 1691, SEQ ID NO: 1692, SEQ ID NO: 1693, SEQ ID NO: 1694, SEQ ID
NO: 1695, SEQ ID NO: 1696, SEQ ID NO: 1697, SEQ ID NO: 1500, SEQ ID NO: 1698, SEQ ID NO: 1503, SEQ ID
NO: 1699, SEQ ID NO: 1700, SEQ ID NO: 1701|CD66D, CD66E, CEA, CEACAM3, CEACAM5, CGM1|31, 297|
SEQ ID NO: 155|11|SEQ ID NO: 1462, SEQ ID NO: 1464, SEQ ID NO: 1465, SEQ ID NO: 1466, SEQ ID
NO: 1674, SEQ ID NO: 1675, SEQ ID NO: 1676, SEQ ID NO: 1677, SEQ ID NO: 1472, SEQ ID NO: 1678, SEQ ID
NO: 1475, SEQ ID NO: 1679, SEQ ID NO: 1680, SEQ ID NO: 1478, SEQ ID NO: 1482, SEQ ID NO: 1483, SEQ ID
NO: 1681, SEQ ID NO: 1487, SEQ ID NO: 1488, SEQ ID NO: 1489, SEQ ID NO: 1493, SEQ ID NO: 1682, SEQ ID
NO: 1683, SEQ ID NO: 1684, SEQ ID NO: 1685, SEQ ID NO: 1686, SEQ ID NO: 1687, SEQ ID NO: 1688, SEQ ID
NO: 1689, SEQ ID NO: 1690, SEQ ID NO: 1691, SEQ ID NO: 1692, SEQ ID NO: 1693, SEQ ID NO: 1694, SEQ ID
NO: 1695, SEQ ID NO: 1696, SEQ ID NO: 1697, SEQ ID NO: 1500, SEQ ID NO: 1698, SEQ ID NO: 1503, SEQ ID
NO: 1699, SEQ ID NO: 1700, SEQ ID NO: 1701|CD66D, CD66E, CEA, CEACAM3, CEACAM5, CGM1|31, 297|
SEQ ID NO: 156|11|SEQ ID NO: 1462, SEQ ID NO: 1464, SEQ ID NO: 1465, SEQ ID NO: 1466, SEQ ID
NO: 1674, SEQ ID NO: 1675, SEQ ID NO: 1676, SEQ ID NO: 1677, SEQ ID NO: 1472, SEQ ID NO: 1678, SEQ ID
NO: 1475, SEQ ID NO: 1679, SEQ ID NO: 1680, SEQ ID NO: 1478, SEQ ID NO: 1482, SEQ ID NO: 1483, SEQ ID
NO: 1681, SEQ ID NO: 1487, SEQ ID NO: 1488, SEQ ID NO: 1489, SEQ ID NO: 1493, SEQ ID NO: 1682, SEQ ID
NO: 1683, SEQ ID NO: 1684, SEQ ID NO: 1685, SEQ ID NO: 1686, SEQ ID NO: 1687, SEQ ID NO: 1688, SEQ ID
NO: 1689, SEQ ID NO: 1690, SEQ ID NO: 1691, SEQ ID NO: 1692, SEQ ID NO: 1693, SEQ ID NO: 1694, SEQ ID
NO: 1695, SEQ ID NO: 1696, SEQ ID NO: 1697, SEQ ID NO: 1500, SEQ ID NO: 1698, SEQ ID NO: 1503, SEQ ID
NO: 1699, SEQ ID NO: 1700, SEQ ID NO: 1701|CD66D, CD66E, CEA, CEACAM3, CEACAM5, CGM1|31, 297|
SEQ ID NO: 157|11|SEQ ID NO: 1462, SEQ ID NO: 1464, SEQ ID NO: 1465, SEQ ID NO: 1466, SEQ ID
NO: 1674, SEQ ID NO: 1675, SEQ ID NO: 1676, SEQ ID NO: 1677, SEQ ID NO: 1472, SEQ ID NO: 1678, SEQ ID
NO: 1475, SEQ ID NO: 1679, SEQ ID NO: 1680, SEQ ID NO: 1478, SEQ ID NO: 1482, SEQ ID NO: 1483, SEQ ID
NO: 1681, SEQ ID NO: 1487, SEQ ID NO: 1488, SEQ ID NO: 1489, SEQ ID NO: 1493, SEQ ID NO: 1682, SEQ ID
NO: 1683, SEQ ID NO: 1684, SEQ ID NO: 1685, SEQ ID NO: 1686, SEQ ID NO: 1687, SEQ ID NO: 1688, SEQ ID
NO: 1689, SEQ ID NO: 1690, SEQ ID NO: 1691, SEQ ID NO: 1692, SEQ ID NO: 1693, SEQ ID NO: 1694, SEQ ID
NO: 1695, SEQ ID NO: 1696, SEQ ID NO: 1697, SEQ ID NO: 1500, SEQ ID NO: 1698, SEQ ID NO: 1503, SEQ ID
NO: 1699, SEQ ID NO: 1700, SEQ ID NO: 1701|CD66D, CD66E, CEA, CEACAM3, CEACAM5, CGM1|31, 297|
SEQ ID NO: 158|11|SEQ ID NO: 1462, SEQ ID NO: 1464, SEQ ID NO: 1465, SEQ ID NO: 1466, SEQ ID
NO: 1467, SEQ ID NO: 1468, SEQ ID NO: 1472, SEQ ID NO: 1475, SEQ ID NO: 1476, SEQ ID NO: 1478, SEQ ID
NO: 1479, SEQ ID NO: 1482, SEQ ID NO: 1483, SEQ ID NO: 1484, SEQ ID NO: 1485, SEQ ID NO: 1487, SEQ ID
NO: 1488, SEQ ID NO: 1489, SEQ ID NO: 1490, SEQ ID NO: 1491, SEQ ID NO: 1493, SEQ ID NO: 1496, SEQ ID
NO: 1500, SEQ ID NO: 1503, SEQ ID NO: 1507|CD66E, CEA, CEACAM5|31, 297|
SEQ ID NO: 159|11|SEQ ID NO: 1462, SEQ ID NO: 1464, SEQ ID NO: 1465, SEQ ID NO: 1466, SEQ ID
NO: 1467, SEQ ID NO: 1468, SEQ ID NO: 1472, SEQ ID NO: 1475, SEQ ID NO: 1476, SEQ ID NO: 1478, SEQ ID
NO: 1479, SEQ ID NO: 1482, SEQ ID NO: 1483, SEQ ID NO: 1484, SEQ ID NO: 1485, SEQ ID NO: 1487, SEQ ID
NO: 1488, SEQ ID NO: 1489, SEQ ID NO: 1490, SEQ ID NO: 1491, SEQ ID NO: 1493, SEQ ID NO: 1496, SEQ ID
NO: 1500, SEQ ID NO: 1503, SEQ ID NO: 1507|CD66E, CEA, CEACAM5|31, 297|
SEQ ID NO: 160|6|SEQ ID NO: 1242, SEQ ID NO: 1462, SEQ ID NO: 1463, SEQ ID NO: 1464, SEQ ID
NO: 1465, SEQ ID NO: 1466, SEQ ID NO: 1467, SEQ ID NO: 1468, SEQ ID NO: 1243, SEQ ID NO: 1469, SEQ ID
NO: 1470, SEQ ID NO: 1471, SEQ ID NO: 1472, SEQ ID NO: 1473, SEQ ID NO: 1474, SEQ ID NO: 1475, SEQ ID
NO: 1476, SEQ ID NO: 1478, SEQ ID NO: 1479, SEQ ID NO: 1480, SEQ ID NO: 1482, SEQ ID NO: 1244, SEQ ID
NO: 1483, SEQ ID NO: 1484, SEQ ID NO: 1485, SEQ ID NO: 1486, SEQ ID NO: 1487, SEQ ID NO: 1488, SEQ ID
NO: 1489, SEQ ID NO: 1490, SEQ ID NO: 1491, SEQ ID NO: 1492, SEQ ID NO: 1493, SEQ ID NO: 1495, SEQ ID
NO: 1496, SEQ ID NO: 1497, SEQ ID NO: 1500, SEQ ID NO: 1501, SEQ ID NO: 1502, SEQ ID NO: 1503, SEQ ID
NO: 1504, SEQ ID NO: 1505, SEQ ID NO: 1506, SEQ ID NO: 1507, SEQ ID NO: 1245|CD66B, CD66C, CD66E,
CD67, CEA, CEACAM5, CEACAM6, CEACAM8, CGM6, NCA, NCA-95|31, 34, 89, 120, 127, 129, 154, 297, 389, 438|
SEQ ID NO: 161|8, 15|SEQ ID NO: 1320, SEQ ID NO: 1321, SEQ ID NO: 1322, SEQ ID NO: 1323, SEQ ID
NO: 1325, SEQ ID NO: 1326, SEQ ID NO: 1327, SEQ ID NO: 1328, SEQ ID NO: 1329, SEQ ID NO: 1330, SEQ ID
NO: 1331, SEQ ID NO: 1332, SEQ ID NO: 1333, SEQ ID NO: 1336, SEQ ID NO: 1337, SEQ ID NO: 1338|37LRP,
LAMBR, LAMR1, LRP, P40, RPSA|31, 34, 36, 72, 162, 186, 352, 386, 395, 396, 397|
SEQ ID NO: 162|15|SEQ ID NO: 1702, SEQ ID NO: 1703, SEQ ID NO: 1704, SEQ ID NO: 1705, SEQ ID
NO: 1706, SEQ ID NO: 1707, SEQ ID NO: 1708, SEQ ID NO: 1709, SEQ ID NO: 1710, SEQ ID NO: 1711, SEQ ID
NO: 1712, SEQ ID NO: 1713|5F7, BSG, CD147, OK|89, 242, 386, 739|
SEQ ID NO: 163|15|SEQ ID NO: 988, SEQ ID NO: 989, SEQ ID NO: 990, SEQ ID NO: 991, SEQ ID NO: 992,
SEQ ID NO: 993, SEQ ID NO: 994, SEQ ID NO: 995, SEQ ID NO: 996, SEQ ID NO: 998, SEQ ID NO: 999, SEQ
ID NO: 1000, SEQ ID NO: 1001, SEQ ID NO: 1002, SEQ ID NO: 1003, SEQ ID NO: 1004, SEQ ID NO: 1005|
CALLA, CD10, MME|31, 36, 47, 49, 51, 120, 154, 282, 389, 640|
SEQ ID NO: 164|15|SEQ ID NO: 1321, SEQ ID NO: 1322, SEQ ID NO: 1323, SEQ ID NO: 1324, SEQ ID
NO: 1325, SEQ ID NO: 1326, SEQ ID NO: 1327, SEQ ID NO: 1328, SEQ ID NO: 1329, SEQ ID NO: 1330, SEQ ID
NO: 1331, SEQ ID NO: 1332, SEQ ID NO: 1333, SEQ ID NO: 1444, SEQ ID NO: 1445, SEQ ID NO: 1446, SEQ ID
NO: 1447, SEQ ID NO: 1336, SEQ ID NO: 1337, SEQ ID NO: 1338, SEQ ID NO: 1339|37LRP, LAMBR, LAMR1, LRP,
P40, RPSA|31, 34, 36, 72, 162, 186, 352, 386, 395, 396, 397|
SEQ ID NO: 165|15|SEQ ID NO: 1714, SEQ ID NO: 1715, SEQ ID NO: 1716, SEQ ID NO: 1717, SEQ ID
NO: 1718, SEQ ID NO: 1719, SEQ ID NO: 1720, SEQ ID NO: 1721, SEQ ID NO: 1722, SEQ ID NO: 1723, SEQ ID
NO: 1724, SEQ ID NO: 1725, SEQ ID NO: 1726|BA2, CD9, MIC3, MRX50, P24|31, 129, 154|
SEQ ID NO: 166|15|SEQ ID NO: 1321, SEQ ID NO: 1322, SEQ ID NO: 1323, SEQ ID NO: 1324, SEQ ID
NO: 1325, SEQ ID NO: 1326, SEQ ID NO: 1327, SEQ ID NO: 1328, SEQ ID NO: 1329, SEQ ID NO: 1330, SEQ ID
NO: 1331, SEQ ID NO: 1332, SEQ ID NO: 1333, SEQ ID NO: 1444, SEQ ID NO: 1445, SEQ ID NO: 1446, SEQ ID
NO: 1447, SEQ ID NO: 1336, SEQ ID NO: 1337, SEQ ID NO: 1338, SEQ ID NO: 1339|37LRP, LAMBR, LAMR1, LRP,
P40, RPSA|31, 34, 36, 72, 162, 186, 352, 386, 395, 396, 397|
SEQ ID NO: 167|15|SEQ ID NO: 1727, SEQ ID NO: 1728, SEQ ID NO: 1729, SEQ ID NO: 1730, SEQ ID
NO: 1731, SEQ ID NO: 1732, SEQ ID NO: 1733, SEQ ID NO: 1734|HPR6.6, MPR, PGRMC1|31, 34, 1180|
SEQ ID NO: 168|13|SEQ ID NO: 1735, SEQ ID NO: 1736, SEQ ID NO: 1737, SEQ ID NO: 1738, SEQ ID
NO: 1739, SEQ ID NO: 1740, SEQ ID NO: 1741, SEQ ID NO: 1742, SEQ ID NO: 1743, SEQ ID NO: 1744|CD11A,
ITGAL, LFA-1, LFA1A|34, 161, 186, 201, 405, 408|
SEQ ID NO: 169|13|SEQ ID NO: 1745, SEQ ID NO: 1746, SEQ ID NO: 1747, SEQ ID NO: 1748, SEQ ID
NO: 1749, SEQ ID NO: 1750, SEQ ID NO: 1751, SEQ ID NO: 1752, SEQ ID NO: 1753|ADAM17, CD156B, CSVP,
TACE|31, 47, 51, 120, 242, 389, 640|
SEQ ID NO: 170|15|SEQ ID NO: 682, SEQ ID NO: 683, SEQ ID NO: 684, SEQ ID NO: 685, SEQ ID NO: 686,
SEQ ID NO: 687, SEQ ID NO: 688|CD71, TFR, TFRC|31, 331, 590, 720, 721, 722|
SEQ ID NO: 171|15|SEQ ID NO: 978, SEQ ID NO: 979, SEQ ID NO: 980, SEQ ID NO: 981, SEQ ID NO: 982,
SEQ ID NO: 983, SEQ ID NO: 985, SEQ ID NO: 986, SEQ ID NO: 987|CD222, CIMPR, IGF2R, M6P-R, MPRI|2, 31,
34, 89, 219, 331, 385, 839, 932|
SEQ ID NO: 172|15|SEQ ID NO: 1320, SEQ ID NO: 1321, SEQ ID NO: 1322, SEQ ID NO: 1323, SEQ ID
NO: 1324, SEQ ID NO: 1325, SEQ ID NO: 1326, SEQ ID NO: 1327, SEQ ID NO: 1328, SEQ ID NO: 1329, SEQ ID
NO: 1330, SEQ ID NO: 1331, SEQ ID NO: 1332, SEQ ID NO: 1333, SEQ ID NO: 1334, SEQ ID NO: 1335, SEQ ID
NO: 1754, SEQ ID NO: 1336, SEQ ID NO: 1337, SEQ ID NO: 1338, SEQ ID NO: 1339|37LRP, LAMBR, LAMR1, LRP,
P40, RPSA|31, 34, 36, 72, 162, 186, 352, 386, 395, 396, 397|
SEQ ID NO: 173|8, 15|SEQ ID NO: 1321, SEQ ID NO: 1322, SEQ ID NO: 1323, SEQ ID NO: 1324, SEQ ID
NO: 1325, SEQ ID NO: 1326, SEQ ID NO: 1327, SEQ ID NO: 1328, SEQ ID NO: 1329, SEQ ID NO: 1330, SEQ ID
NO: 1331, SEQ ID NO: 1332, SEQ ID NO: 1333, SEQ ID NO: 1444, SEQ ID NO: 1445, SEQ ID NO: 1446, SEQ ID
NO: 1447, SEQ ID NO: 1336, SEQ ID NO: 1337, SEQ ID NO: 1338, SEQ ID NO: 1339|37LRP, LAMBR, LAMR1, LRP,
P40, RPSA|31, 34, 36, 72, 162, 186, 352, 386, 395, 396, 397|
SEQ ID NO: 174|8, 15|SEQ ID NO: 1321, SEQ ID NO: 1322, SEQ ID NO: 1323, SEQ ID NO: 1324, SEQ ID
NO: 1325, SEQ ID NO: 1326, SEQ ID NO: 1327, SEQ ID NO: 1328, SEQ ID NO: 1330, SEQ ID NO: 1331, SEQ ID
NO: 1332, SEQ ID NO: 1755, SEQ ID NO: 1336, SEQ ID NO: 1337, SEQ ID NO: 1338, SEQ ID NO: 1339|37LRP,
LAMBR, LAMR1, LRP, P40, RPSA|31, 34, 36, 72, 162, 186, 352, 386, 395, 396, 397|
SEQ ID NO: 175|13|SEQ ID NO: 1756, SEQ ID NO: 1757, SEQ ID NO: 1758, SEQ ID NO: 1759, SEQ ID
NO: 1760, SEQ ID NO: 1761, SEQ ID NO: 1762, SEQ ID NO: 1763, SEQ ID NO: 1764, SEQ ID NO: 1765, SEQ ID
NO: 1766, SEQ ID NO: 1767, SEQ ID NO: 1768, SEQ ID NO: 1769, SEQ ID NO: 1770, SEQ ID NO: 1771, SEQ ID
NO: 1772, SEQ ID NO: 1773|IFNAR, IFNAR1, IFRC|31, 34, 129, 229, 263, 386, 1247|
SEQ ID NO: 176|13|SEQ ID NO: 929, SEQ ID NO: 930, SEQ ID NO: 931, SEQ ID NO: 932, SEQ ID NO: 933,
SEQ ID NO: 1774, SEQ ID NO: 934, SEQ ID NO: 935, SEQ ID NO: 936, SEQ ID NO: 937, SEQ ID NO: 938, SEQ
ID NO: 939|CD49F, ITGA6|34, 200, 224, 405, 408, 923|
SEQ ID NO: 177|13|SEQ ID NO: 1775, SEQ ID NO: 1776, SEQ ID NO: 1777, SEQ ID NO: 1778, SEQ ID
NO: 1779, SEQ ID NO: 1780, SEQ ID NO: 1781, SEQ ID NO: 1782, SEQ ID NO: 1783, SEQ ID NO: 1784|CTNNB,
CTNNB1|1, 2, 8, 10, 16, 36, 89, 151, 154, 186, 790|
SEQ ID NO: 178|13|SEQ ID NO: 1785, SEQ ID NO: 1786, SEQ ID NO: 1787, SEQ ID NO: 1788, SEQ ID
NO: 1789, SEQ ID NO: 1790, SEQ ID NO: 1791, SEQ ID NO: 1792, SEQ ID NO: 1793, SEQ ID NO: 1794, SEQ ID
NO: 1795, SEQ ID NO: 1796|CD98, D16S469E, E16, LAT1, MPE16, SLC7A5|29, 111, 154, 712, 777, 1189|
SEQ ID NO: 179|13|SEQ ID NO: 1797, SEQ ID NO: 1798, SEQ ID NO: 1799, SEQ ID NO: 1800, SEQ ID
NO: 1801, SEQ ID NO: 1802|NKTR|129, 154, 254, 255, 773|
SEQ ID NO: 180|13|SEQ ID NO: 1803, SEQ ID NO: 1804, SEQ ID NO: 1805, SEQ ID NO: 1806, SEQ ID
NO: 1807, SEQ ID NO: 1808, SEQ ID NO: 1809, SEQ ID NO: 1810, SEQ ID NO: 1811, SEQ ID NO: 1812, SEQ ID
NO: 1813, SEQ ID NO: 1814|DCR3, DJ583P15.1.1, DKFZP434C013, KIAA1088, M68, NHL, TNFRSF6B, TR6|18, 34,
36, 68, 81, 138, 176, 179, 385|
SEQ ID NO: 181|13|SEQ ID NO: 1815, SEQ ID NO: 1816, SEQ ID NO: 1817, SEQ ID NO: 1818, SEQ ID
NO: 1819, SEQ ID NO: 1820, SEQ ID NO: 1821, SEQ ID NO: 1822|ARAF1, PKS2, RAFA1|12, 36, 104, 166|
SEQ ID NO: 182|13|SEQ ID NO: 1823, SEQ ID NO: 1824, SEQ ID NO: 1825, SEQ ID NO: 1826, SEQ ID
NO: 1827, SEQ ID NO: 1828, SEQ ID NO: 1829, SEQ ID NO: 1830, SEQ ID NO: 1831, SEQ ID NO: 1832, SEQ ID
NO: 1833, SEQ ID NO: 1834, SEQ ID NO: 1835|EIF3-P46, EIF3-P48, EIF3S6, INT6|72, 231, 336, 391, 491,
492|
SEQ ID NO: 183|13|SEQ ID NO: 1775, SEQ ID NO: 1776, SEQ ID NO: 1777, SEQ ID NO: 1778, SEQ ID
NO: 1779, SEQ ID NO: 1780, SEQ ID NO: 1782, SEQ ID NO: 1783, SEQ ID NO: 1784|CTNNB, CTNNB1|1, 2, 8, 10,
16, 36, 89, 151, 154, 186, 790|
SEQ ID NO: 184|13|SEQ ID NO: 1836, SEQ ID NO: 1837, SEQ ID NO: 1838, SEQ ID NO: 1839, SEQ ID
NO: 1840, SEQ ID NO: 1841, SEQ ID NO: 1842|ERM, ETV5|3, 7, 23|
SEQ ID NO: 185|13|SEQ ID NO: 1843, SEQ ID NO: 1844, SEQ ID NO: 1845, SEQ ID NO: 1846, SEQ ID
NO: 1847, SEQ ID NO: 1848, SEQ ID NO: 1849, SEQ ID NO: 1850, SEQ ID NO: 1851, SEQ ID NO: 1852, SEQ ID
NO: 1853, SEQ ID NO: 1854|KIAA0619, P160ROCK, ROCK1, ROCK2|12, 87, 89, 104, 105, 131, 132, 133, 134, 320,
502, 581, 583, 678|
SEQ ID NO: 186|13|SEQ ID NO: 1855, SEQ ID NO: 1856, SEQ ID NO: 1857, SEQ ID NO: 1858, SEQ ID
NO: 1859, SEQ ID NO: 1860, SEQ ID NO: 1861|PFN1|134, 137, 857|
SEQ ID NO: 187|14|SEQ ID NO: 682, SEQ ID NO: 683, SEQ ID NO: 684, SEQ ID NO: 685, SEQ ID NO: 686,
SEQ ID NO: 1862, SEQ ID NO: 1863, SEQ ID NO: 1864, SEQ ID NO: 687, SEQ ID NO: 688|CD71, TFR, TFRC|31,
331, 590, 720, 721, 722|
SEQ ID NO: 188|14|SEQ ID NO: 929, SEQ ID NO: 930, SEQ ID NO: 931, SEQ ID NO: 932, SEQ ID NO: 933,
SEQ ID NO: 934, SEQ ID NO: 935, SEQ ID NO: 936, SEQ ID NO: 937, SEQ ID NO: 938, SEQ ID NO: 939|CD49F,
ITGA6|34, 200, 224, 405, 408, 923|
SEQ ID NO: 189|15|SEQ ID NO: 1865, SEQ ID NO: 1866, SEQ ID NO: 1867, SEQ ID NO: 1868, SEQ ID
NO: 1869, SEQ ID NO: 1870, SEQ ID NO: 1871, SEQ ID NO: 1872, SEQ ID NO: 1873, SEQ ID NO: 1874, SEQ ID
NO: 1875, SEQ ID NO: 1876, SEQ ID NO: 1877, SEQ ID NO: 1878, SEQ ID NO: 1879, SEQ ID NO: 1880, SEQ ID
NO: 1881, SEQ ID NO: 1882|GLBA, PSAP, SAP1|17, 209, 234, 369, 377, 438, 464, 1181|
SEQ ID NO: 190|15|SEQ ID NO: 1462, SEQ ID NO: 1464, SEQ ID NO: 1466, SEQ ID NO: 1468, SEQ ID
NO: 1472, SEQ ID NO: 1475, SEQ ID NO: 1476, SEQ ID NO: 1478, SEQ ID NO: 1479, SEQ ID NO: 1482, SEQ ID
NO: 1483, SEQ ID NO: 1485, SEQ ID NO: 1487, SEQ ID NO: 1488, SEQ ID NO: 1489, SEQ ID NO: 1491, SEQ ID
NO: 1493, SEQ ID NO: 1496, SEQ ID NO: 1500, SEQ ID NO: 1503|CD66E, CEA, CEACAM5|31, 297|
SEQ ID NO: 191|15|SEQ ID NO: 1340, SEQ ID NO: 1341, SEQ ID NO: 1342, SEQ ID NO: 1343, SEQ ID
NO: 1344, SEQ ID NO: 1345, SEQ ID NO: 1346, SEQ ID NO: 1347, SEQ ID NO: 1348, SEQ ID NO: 1350, SEQ ID
NO: 1351, SEQ ID NO: 1352, SEQ ID NO: 1353|GALBP, LGALS2, LGALS3, MAC-2, MAC2|56, 154, 635, 1012|
SEQ ID NO: 192|13|SEQ ID NO: 1883, SEQ ID NO: 1462, SEQ ID NO: 1464, SEQ ID NO: 1465, SEQ ID
NO: 1466, SEQ ID NO: 1467, SEQ ID NO: 1468, SEQ ID NO: 1884, SEQ ID NO: 1472, SEQ ID NO: 1885, SEQ ID
NO: 1886, SEQ ID NO: 1887, SEQ ID NO: 1475, SEQ ID NO: 1679, SEQ ID NO: 1476, SEQ ID NO: 1478, SEQ ID
NO: 1888, SEQ ID NO: 1479, SEQ ID NO: 1889, SEQ ID NO: 1482, SEQ ID NO: 1483, SEQ ID NO: 1484, SEQ ID
NO: 1485, SEQ ID NO: 1487, SEQ ID NO: 1488, SEQ ID NO: 1489, SEQ ID NO: 1490, SEQ ID NO: 1491, SEQ ID
NO: 1493, SEQ ID NO: 1890, SEQ ID NO: 1891, SEQ ID NO: 1495, SEQ ID NO: 1496, SEQ ID NO: 1892, SEQ ID
NO: 1893, SEQ ID NO: 1500, SEQ ID NO: 1894, SEQ ID NO: 1503, SEQ ID NO: 1895, SEQ ID NO: 1896, SEQ ID
NO: 1507|CD66E, CEA, CEACAM5, CEACAM7, CGM2|2, 31, 154, 209, 297|
SEQ ID NO: 193|15|SEQ ID NO: 1340, SEQ ID NO: 1341, SEQ ID NO: 1342, SEQ ID NO: 1343, SEQ ID
NO: 1344, SEQ ID NO: 1345, SEQ ID NO: 1346, SEQ ID NO: 1347, SEQ ID NO: 1348, SEQ ID NO: 1350, SEQ ID
NO: 1448, SEQ ID NO: 1351, SEQ ID NO: 1352, SEQ ID NO: 1353|GALBP, LGALS2, LGALS3, MAC-2, MAC2|56,
154, 635, 1012|
SEQ ID NO: 194|15|SEQ ID NO: 929, SEQ ID NO: 930, SEQ ID NO: 931, SEQ ID NO: 932, SEQ ID NO: 933,
SEQ ID NO: 1774, SEQ ID NO: 934, SEQ ID NO: 935, SEQ ID NO: 936, SEQ ID NO: 937, SEQ ID NO: 938, SEQ
ID NO: 939|CD49F, ITGA6|34, 200, 224, 405, 408, 923|
SEQ ID NO: 195|15|SEQ ID NO: 1897, SEQ ID NO: 1898, SEQ ID NO: 1899|ALCAM, CD166, MEMD|31, 89, 125,
129, 141, 147, 186, 187, 387|
SEQ ID NO: 196|15|SEQ ID NO: 1242, SEQ ID NO: 1462, SEQ ID NO: 1463, SEQ ID NO: 1464, SEQ ID
NO: 1465, SEQ ID NO: 1466, SEQ ID NO: 1676, SEQ ID NO: 1243, SEQ ID NO: 1470, SEQ ID NO: 1900, SEQ ID
NO: 1472, SEQ ID NO: 1901, SEQ ID NO: 1475, SEQ ID NO: 1679, SEQ ID NO: 1476, SEQ ID NO: 1477, SEQ ID
NO: 1478, SEQ ID NO: 1480, SEQ ID NO: 1482, SEQ ID NO: 1244, SEQ ID NO: 1483, SEQ ID NO: 1902, SEQ ID
NO: 1485, SEQ ID NO: 1486, SEQ ID NO: 1487, SEQ ID NO: 1903, SEQ ID NO: 1488, SEQ ID NO: 1489, SEQ ID
NO: 1491, SEQ ID NO: 1492, SEQ ID NO: 1493, SEQ ID NO: 1496, SEQ ID NO: 1497, SEQ ID NO: 1498, SEQ ID
NO: 1500, SEQ ID NO: 1502, SEQ ID NO: 1503, SEQ ID NO: 1505, SEQ ID NO: 1700, SEQ ID NO: 1506, SEQ ID
NO: 1904, SEQ ID NO: 1245|CD66C, CD66E, CEA, CEACAM, CEACAM6, NCA|31, 34, 89, 120, 297, 389|
SEQ ID NO: 197|15|SEQ ID NO: 1340, SEQ ID NO: 1341, SEQ ID NO: 1342, SEQ ID NO: 1343, SEQ ID
NO: 1344, SEQ ID NO: 1345, SEQ ID NO: 1346, SEQ ID NO: 1347, SEQ ID NO: 1348, SEQ ID NO: 1349, SEQ ID
NO: 1350, SEQ ID NO: 1351, SEQ ID NO: 1352, SEQ ID NO: 1353|GALBP, LGALS2, LGALS3, MAC-2, MAC2|56,
154, 635, 1012|
SEQ ID NO: 198|15|SEQ ID NO: 1702, SEQ ID NO: 1703, SEQ ID NO: 1704, SEQ ID NO: 1705, SEQ ID
NO: 1706, SEQ ID NO: 1707, SEQ ID NO: 1708, SEQ ID NO: 1709, SEQ ID NO: 1710, SEQ ID NO: 1711, SEQ ID
NO: 1712, SEQ ID NO: 1713|5F7, BSG, CD147, OK|89, 242, 386, 739|
SEQ ID NO: 199|8|SEQ ID NO: 1905, SEQ ID NO: 1906, SEQ ID NO: 1907, SEQ ID NO: 1908, SEQ ID
NO: 1909, SEQ ID NO: 1910, SEQ ID NO: 1911, SEQ ID NO: 1912|CD45, GP180, LCA, PTPRC, T200|31, 34, 47,
386, 469, 858|
SEQ ID NO: 200|13|SEQ ID NO: 1745, SEQ ID NO: 1746, SEQ ID NO: 1913, SEQ ID NO: 1747, SEQ ID
NO: 1748, SEQ ID NO: 1749, SEQ ID NO: 1914, SEQ ID NO: 1750, SEQ ID NO: 1751, SEQ ID NO: 1915, SEQ ID
NO: 1752, SEQ ID NO: 1916, SEQ ID NO: 1753|ADAM17, CD156B, CSVP, TACE|31, 47, 51, 120, 242, 389, 640|
SEQ ID NO: 201|8|SEQ ID NO: 256, SEQ ID NO: 257, SEQ ID NO: 258, SEQ ID NO: 259, SEQ ID NO: 260, SEQ
ID NO: 261, SEQ ID NO: 262, SEQ ID NO: 263, SEQ ID NO: 264|CD27L, CD27LG, CD70, KI-24 ANTIGEN,
TNFSF7|31, 56, 57, 89, 120, 179, 387, 388, 389|
SEQ ID NO: 202|8|SEQ ID NO: 256, SEQ ID NO: 257, SEQ ID NO: 258, SEQ ID NO: 259, SEQ ID NO: 260, SEQ
ID NO: 261, SEQ ID NO: 262, SEQ ID NO: 263, SEQ ID NO: 264|CD27L, CD27LG, CD70, KI-24 ANTIGEN,
TNFSF7|31, 56, 57, 89, 120, 179, 387, 388, 389|
SEQ ID NO: 203|8|SEQ ID NO: 1515, SEQ ID NO: 1516, SEQ ID NO: 1517, SEQ ID NO: 1518, SEQ ID
NO: 1519, SEQ ID NO: 1520, SEQ ID NO: 1521, SEQ ID NO: 1522, SEQ ID NO: 1523, SEQ ID NO: 1524, SEQ ID
NO: 1526, SEQ ID NO: 1527, SEQ ID NO: 1528, SEQ ID NO: 1529, SEQ ID NO: 1530, SEQ ID NO: 1531, SEQ ID
NO: 1532, SEQ ID NO: 1533, SEQ ID NO: 1534, SEQ ID NO: 1535|B1, BP35, CD20, MS4A1, MS4A1A, MS4A2|22,
31, 57, 129, 359, 789, 1110|
SEQ ID NO: 204|8|SEQ ID NO: 885, SEQ ID NO: 886, SEQ ID NO: 887, SEQ ID NO: 888, SEQ ID NO: 889, SEQ
ID NO: 890, SEQ ID NO: 891, SEQ ID NO: 892, SEQ ID NO: 893, SEQ ID NO: 894, SEQ ID NO: 895, SEQ ID
NO: 896, SEQ ID NO: 897, SEQ ID NO: 898, SEQ ID NO: 899, SEQ ID NO: 900, SEQ ID NO: 901, SEQ ID
NO: 902, SEQ ID NO: 903, SEQ ID NO: 904|CD23, CD23A, FCE2, FCER2, SST|28, 31, 33, 34, 55, 56, 102, 120,
129, 179, 253, 359, 386, 389, 438, 765, 848, 849, 850, 851|
SEQ ID NO: 205|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 505, SEQ ID NO: 506, SEQ ID NO: 507, SEQ ID NO: 508, SEQ ID
NO: 509, SEQ ID NO: 510|4F2, 4F2HC, CD98, MDU1, NACAE, SLC3A2|
SEQ ID NO: 206|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 1917, SEQ ID NO: 1918, SEQ ID NO: 505, SEQ ID NO: 506, SEQ ID
NO: 507, SEQ ID NO: 508, SEQ ID NO: 509, SEQ ID NO: 510|4F2, 4F2HC, CD98, MDU1, NACAE, SLC3A2|
SEQ ID NO: 207|8|SEQ ID NO: 1515, SEQ ID NO: 1516, SEQ ID NO: 1517, SEQ ID NO: 1518, SEQ ID
NO: 1519, SEQ ID NO: 1919, SEQ ID NO: 1920, SEQ ID NO: 1520, SEQ ID NO: 1521, SEQ ID NO: 1522, SEQ ID
NO: 1523, SEQ ID NO: 1921, SEQ ID NO: 1524, SEQ ID NO: 1525, SEQ ID NO: 1526, SEQ ID NO: 1527, SEQ ID
NO: 1528, SEQ ID NO: 1529, SEQ ID NO: 1530, SEQ ID NO: 1531, SEQ ID NO: 1533, SEQ ID NO: 1534, SEQ ID
NO: 1535|B1, BP35, CD20, MS4A1, MS4A1A, MS4A2|22, 31, 57, 129, 359, 789, 1110|
SEQ ID NO: 208|8|SEQ ID NO: 1922, SEQ ID NO: 1923, SEQ ID NO: 1924, SEQ ID NO: 1925, SEQ ID
NO: 1926, SEQ ID NO: 1927, SEQ ID NO: 1928, SEQ ID NO: 1929, SEQ ID NO: 1930, SEQ ID NO: 1931, SEQ ID
NO: 1932|4F9, C33, CD82, GR15, IA4, KAI1, PREB, R2, SAR2, ST6|2, 8, 10, 14, 16, 17, 31, 129, 154|
SEQ ID NO: 209|8|SEQ ID NO: 1933, SEQ ID NO: 1934, SEQ ID NO: 1935, SEQ ID NO: 1936, SEQ ID
NO: 1937, SEQ ID NO: 1938, SEQ ID NO: 1939, SEQ ID NO: 1940, SEQ ID NO: 1941, SEQ ID NO: 1942, SEQ ID
NO: 1943, SEQ ID NO: 1944|CD74, DHLAG|129, 209, 413|
SEQ ID NO: 210|8|SEQ ID NO: 498, SEQ ID NO: 499, SEQ ID NO: 500, SEQ ID NO: 501, SEQ ID NO: 503, SEQ
ID NO: 504, SEQ ID NO: 505, SEQ ID NO: 506, SEQ ID NO: 507, SEQ ID NO: 508, SEQ ID NO: 510|4F2, 4F2HC,
CD98, MDU1, NACAE, SLC3A2|
SEQ ID NO: 211|8|SEQ ID NO: 708, SEQ ID NO: 709, SEQ ID NO: 710, SEQ ID NO: 711, SEQ ID NO: 712, SEQ
ID NO: 713, SEQ ID NO: 714, SEQ ID NO: 715, SEQ ID NO: 716, SEQ ID NO: 717, SEQ ID NO: 718, SEQ ID
NO: 719, SEQ ID NO: 720|CD18, ITGB2, LAD, LCAMB, LFA-1, MF17|34, 129, 141, 186, 405, 408, 766|
SEQ ID NO: 212|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 1917, SEQ ID NO: 1918, SEQ ID NO: 505, SEQ ID NO: 506, SEQ ID
NO: 507, SEQ ID NO: 508, SEQ ID NO: 509, SEQ ID NO: 510|4F2, 4F2HC, CD98, MDU1, NACAE, SLC3A2|
SEQ ID NO: 213|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 505, SEQ ID NO: 506, SEQ ID NO: 507, SEQ ID NO: 508, SEQ ID
NO: 509, SEQ ID NO: 510|4F2, 4F2HC, CD98, MDU1, NACAE, SLC3A2|
SEQ ID NO: 214|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 505, SEQ ID NO: 506, SEQ ID NO: 507, SEQ ID NO: 508, SEQ ID
NO: 509, SEQ ID NO: 510|4F2, 4F2HC, CD98, MDU1, NACAE, SLC3A2|
SEQ ID NO: 215|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 505, SEQ ID NO: 506, SEQ ID NO: 508, SEQ ID NO: 509, SEQ ID
NO: 510|4F2, 4F2HC, CD98, MDU1, NACAE, SLC3A2|
SEQ ID NO: 216|8|SEQ ID NO: 885, SEQ ID NO: 886, SEQ ID NO: 887, SEQ ID NO: 888, SEQ ID NO: 889, SEQ
ID NO: 890, SEQ ID NO: 891, SEQ ID NO: 893, SEQ ID NO: 895, SEQ ID NO: 896, SEQ ID NO: 901, SEQ ID
NO: 902, SEQ ID NO: 1945, SEQ ID NO: 1946, SEQ ID NO: 903, SEQ ID NO: 904|CD23, CD23A, FCE2, FCER2,
SST|28, 31, 33, 34, 55, 56, 102, 120, 129, 179, 253, 359, 386, 389, 438, 765, 848, 849, 850, 851|
SEQ ID NO: 217|8|SEQ ID NO: 708, SEQ ID NO: 709, SEQ ID NO: 710, SEQ ID NO: 711, SEQ ID NO: 713, SEQ
ID NO: 714, SEQ ID NO: 715, SEQ ID NO: 716, SEQ ID NO: 717, SEQ ID NO: 718, SEQ ID NO: 719, SEQ ID
NO: 720|CD18, ITGB2, LAD, LCAMB, LFA-1, MF17|34, 129, 141, 186, 405, 408, 766|
SEQ ID NO: 218|8|SEQ ID NO: 708, SEQ ID NO: 710, SEQ ID NO: 711, SEQ ID NO: 712, SEQ ID NO: 713, SEQ
ID NO: 714, SEQ ID NO: 715, SEQ ID NO: 716, SEQ ID NO: 717, SEQ ID NO: 718, SEQ ID NO: 719, SEQ ID
NO: 720|CD18, ITGB2, LAD, LCAMB, LFA-1, MF17|34, 129, 141, 186, 405, 408, 766|
SEQ ID NO: 219|8|SEQ ID NO: 682, SEQ ID NO: 683, SEQ ID NO: 684, SEQ ID NO: 685, SEQ ID NO: 922, SEQ
ID NO: 686, SEQ ID NO: 687, SEQ ID NO: 688|CD71, TFR, TFRC|31, 331, 590, 720, 721, 722|
SEQ ID NO: 220|8|SEQ ID NO: 1947, SEQ ID NO: 1948, SEQ ID NO: 1949, SEQ ID NO: 1950, SEQ ID
NO: 1951, SEQ ID NO: 1952, SEQ ID NO: 1953, SEQ ID NO: 1954, SEQ ID NO: 1955, SEQ ID NO: 1956, SEQ ID
NO: 1957, SEQ ID NO: 1958, SEQ ID NO: 1959, SEQ ID NO: 1960, SEQ ID NO: 1961, SEQ ID NO: 1962, SEQ ID
NO: 1963, SEQ ID NO: 1964, SEQ ID NO: 1965, SEQ ID NO: 1966, SEQ ID NO: 1967, SEQ ID NO: 1968, SEQ ID
NO: 1969, SEQ ID NO: 1970|CD28LG, CD28LG1, CD80, LAB7|89, 120, 127, 129, 154, 387|
SEQ ID NO: 221|8|SEQ ID NO: 721, SEQ ID NO: 722, SEQ ID NO: 723, SEQ ID NO: 1971, SEQ ID NO: 725,
SEQ ID NO: 726, SEQ ID NO: 1972, SEQ ID NO: 1973, SEQ ID NO: 728, SEQ ID NO: 729, SEQ ID NO: 730, SEQ
ID NO: 731, SEQ ID NO: 732, SEQ ID NO: 733, SEQ ID NO: 1974, SEQ ID NO: 1975, SEQ ID NO: 734, SEQ ID
NO: 735, SEQ ID NO: 736, SEQ ID NO: 737, SEQ ID NO: 738, SEQ ID NO: 739, SEQ ID NO: 740, SEQ ID
NO: 741, SEQ ID NO: 742, SEQ ID NO: 743, SEQ ID NO: 1976, SEQ ID NO: 1977, SEQ ID NO: 1978, SEQ ID
NO: 1979, SEQ ID NO: 1980, SEQ ID NO: 1981, SEQ ID NO: 1982, SEQ ID NO: 1983, SEQ ID NO: 1984, SEQ ID
NO: 1985, SEQ ID NO: 1986, SEQ ID NO: 1987, SEQ ID NO: 1988, SEQ ID NO: 1989, SEQ ID NO: 1990, SEQ ID
NO: 744, SEQ ID NO: 745, SEQ ID NO: 1991, SEQ ID NO: 1992, SEQ ID NO: 1993, SEQ ID NO: 746, SEQ ID
NO: 747, SEQ ID NO: 1994, SEQ ID NO: 1995, SEQ ID NO: 1996, SEQ ID NO: 1997, SEQ ID NO: 1998, SEQ ID
NO: 1999, SEQ ID NO: 2000, SEQ ID NO: 2001, SEQ ID NO: 748, SEQ ID NO: 749, SEQ ID NO: 750, SEQ ID
NO: 751, SEQ ID NO: 752, SEQ ID NO: 2002, SEQ ID NO: 754|BB2, CD54, ICAM1|129, 154, 186, 187, 219, 231,
331, 385, 717, 778|
SEQ ID NO: 222|8|SEQ ID NO: 2003, SEQ ID NO: 2004, SEQ ID NO: 2005, SEQ ID NO: 2006, SEQ ID
NO: 2007, SEQ ID NO: 2008, SEQ ID NO: 2009, SEQ ID NO: 2010, SEQ ID NO: 2011|CD81, TAPA-1, TAPA1|31,
56, 57, 129, 154, 192, 332|
SEQ ID NO: 223|8|SEQ ID NO: 498, SEQ ID NO: 499, SEQ ID NO: 500, SEQ ID NO: 503, SEQ ID NO: 504, SEQ
ID NO: 505, SEQ ID NO: 506, SEQ ID NO: 508, SEQ ID NO: 510|4F2, 4F2HC, CD98, MDU1, NACAE, SLC3A2|
SEQ ID NO: 224|8|SEQ ID NO: 1905, SEQ ID NO: 1906, SEQ ID NO: 1907, SEQ ID NO: 1908, SEQ ID
NO: 1909, SEQ ID NO: 1910, SEQ ID NO: 1911, SEQ ID NO: 1912|CD45, GP180, LCA, PTPRC, T200|31, 34, 47,
386, 469, 858|
SEQ ID NO: 225|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 505, SEQ ID NO: 506, SEQ ID NO: 508, SEQ ID NO: 509, SEQ ID
NO: 510|4F2, 4F2HC, CD98, MDU1, NACAE, SLC3A2|
SEQ ID NO: 226|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 505, SEQ ID NO: 506, SEQ ID NO: 507, SEQ ID NO: 508, SEQ ID
NO: 509, SEQ ID NO: 510|4F2, 4F2HC, CD98, MDU1, NACAE, SLC3A2|
SEQ ID NO: 227|8|SEQ ID NO: 256, SEQ ID NO: 258, SEQ ID NO: 262, SEQ ID NO: 264|CD27L, CD27LG, CD70,
KI-24 ANTIGEN, TNFSF7|31, 56, 57, 89, 120, 179, 387, 388, 389|
SEQ ID NO: 228|8|SEQ ID NO: 2012, SEQ ID NO: 2013, SEQ ID NO: 2014, SEQ ID NO: 2015, SEQ ID
NO: 2016, SEQ ID NO: 2017, SEQ ID NO: 2018, SEQ ID NO: 2019, SEQ ID NO: 2020, SEQ ID NO: 2021, SEQ ID
NO: 2022, SEQ ID NO: 2023, SEQ ID NO: 2024, SEQ ID NO: 2025, SEQ ID NO: 2026, SEQ ID NO: 2027, SEQ ID
NO: 2028, SEQ ID NO: 2029, SEQ ID NO: 2030, SEQ ID NO: 2031|CD22, SIGLEC-2|31, 120, 129, 141, 186, 187|
SEQ ID NO: 229|8|SEQ ID NO: 1933, SEQ ID NO: 1934, SEQ ID NO: 1935, SEQ ID NO: 1936, SEQ ID
NO: 1937, SEQ ID NO: 1938, SEQ ID NO: 1939, SEQ ID NO: 2032, SEQ ID NO: 1940, SEQ ID NO: 1941, SEQ ID
NO: 1942, SEQ ID NO: 1943, SEQ ID NO: 1944|CD74, DHLAG|129, 209, 413|
SEQ ID NO: 230|8|SEQ ID NO: 1735, SEQ ID NO: 1736, SEQ ID NO: 1737, SEQ ID NO: 1738, SEQ ID
NO: 1739, SEQ ID NO: 1740, SEQ ID NO: 1741, SEQ ID NO: 2033, SEQ ID NO: 1742, SEQ ID NO: 1743, SEQ ID
NO: 1744|CD11A, ITGAL, LFA-1, LFA1A|34, 161, 186, 201, 405, 408|
SEQ ID NO: 231|8|SEQ ID NO: 1905, SEQ ID NO: 1906, SEQ ID NO: 1907, SEQ ID NO: 2034, SEQ ID
NO: 2035, SEQ ID NO: 1908, SEQ ID NO: 2036, SEQ ID NO: 1909, SEQ ID NO: 1910, SEQ ID NO: 1911, SEQ ID
NO: 1912|CD45, GP180, LCA, PTPRC, T200|31, 34, 47, 386, 469, 858|
SEQ ID NO: 232|8|SEQ ID NO: 498, SEQ ID NO: 499, SEQ ID NO: 500, SEQ ID NO: 501, SEQ ID NO: 502, SEQ
ID NO: 503, SEQ ID NO: 504, SEQ ID NO: 505, SEQ ID NO: 506, SEQ ID NO: 507, SEQ ID NO: 508, SEQ ID
NO: 509, SEQ ID NO: 510|4F2, 4F2HC, CD98, MDU1, NACAE, SLC3A2|
SEQ ID NO: 233|8|SEQ ID NO: 1268, SEQ ID NO: 1269, SEQ ID NO: 1270, SEQ ID NO: 1271, SEQ ID
NO: 1272, SEQ ID NO: 1273, SEQ ID NO: 1274, SEQ ID NO: 1275, SEQ ID NO: 1276, SEQ ID NO: 1277, SEQ ID
NO: 1278, SEQ ID NO: 1279, SEQ ID NO: 1280, SEQ ID NO: 2037, SEQ ID NO: 1281, SEQ ID NO: 1282, SEQ ID
NO: 1283, SEQ ID NO: 1284, SEQ ID NO: 1285, SEQ ID NO: 1286, SEQ ID NO: 1287, SEQ ID NO: 1288, SEQ ID
NO: 1289, SEQ ID NO: 1290, SEQ ID NO: 2038, SEQ ID NO: 1291, SEQ ID NO: 1292, SEQ ID NO: 1293, SEQ ID
NO: 2039, SEQ ID NO: 1296, SEQ ID NO: 1297, SEQ ID NO: 1298, SEQ ID NO: 1299, SEQ ID NO: 1300, SEQ ID
NO: 1301, SEQ ID NO: 1302, SEQ ID NO: 1303, SEQ ID NO: 1304, SEQ ID NO: 1305, SEQ ID NO: 1306, SEQ ID
NO: 1307, SEQ ID NO: 1308, SEQ ID NO: 1309, SEQ ID NO: 1310, SEQ ID NO: 1311, SEQ ID NO: 2040, SEQ ID
NO: 1312, SEQ ID NO: 1313, SEQ ID NO: 1314, SEQ ID NO: 1315, SEQ ID NO: 1316, SEQ ID NO: 1317, SEQ ID
NO: 1318, SEQ ID NO: 1319|CD44, CD44R, IN, MC56, MDU2, MDU3, MIC4|
SEQ ID NO: 234|8|SEQ ID NO: 2041|GLBA, PSAP, SAP1|17, 209, 234, 369, 377, 438, 464, 1181|
SEQ ID NO: 235|18|SEQ ID NO: 940, SEQ ID NO: 943, SEQ
ID NO: 945, SEQ ID NO: 948, SEQ ID NO: 949, SEQ ID NO: 950, SEQ ID
NO: 951, SEQ ID NO: 958, SEQ ID NO: 959, SEQ ID NO: 960, SEQ ID NO: 961, SEQ ID NO: 962, SEQ ID
NO: 963, SEQ ID NO: 964, SEQ ID NO: 965, SEQ ID NO: 970, SEQ ID NO: 971, SEQ ID NO: 972|CALM, CLTH,
DJ167A19.1, FLT4, KIAA0656, PCL, PICALM, SNAP91, VEGFR3|12, 31, 34, 135, 332, 411, 461, 639, 815|

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated nucleic acid comprising a nucleotide sequence that encodes a polypeptide comprising an amino acid sequence which is at least 95% identical to an amino acid sequence selected from the group consisting of SEQ ID NOs: 1-235.

2. An isolated nucleic acid comprising a nucleotide sequence that encodes a polypeptide comprising an amino acid sequence selected from the group consisting of SEQ ID NOs: 1-235.

3-9. (Canceled)

10. A purified polypeptide comprising an amino acid sequence selected from the group consisting of SEQ ID NOs: 1-235.

11-14. (Canceled)

15. A purified polypeptide comprising an amino acid sequence selected from the group consisting of SEQ ID NOs: 1-235, wherein the purified polypeptide comprises at least 25 amino acids.

16. The purified polypeptide of claim 14 wherein the polypeptide comprises fewer than 100 amino acids.

17. The purified polypeptide of claim 15 wherein the polypeptide comprises fewer than 50 amino acids.

18-19. (Canceled)

20. A vector comprising the nucleic acid of claim 1.

21. The vector of claim 20 wherein the vector comprises expression control sequences that direct the expression of the polypeptide.

22. The vector of claim 20 wherein the vector comprises expression control sequences that direct expression of the nucleic acid molecule.

23. A cell comprising the vector of claim 20.

24. An antibody that selectively binds a polypeptide consisting of an amino acid sequence selected from the group consisting of SEQ ID NOs:1-235.

25. A method of making an antibody, the method comprising:
(a) providing a polypeptide comprising an amino acid sequence selected from the group consisting of SEQ ID NOs: 1-235 to a mammal in an amount effective to induce the production of an antibody that binds to the polypeptide;
(b) isolating from the mammal a cell that produces an antibody that selectively binds to a polypeptide consisting of an amino acid sequence selected from the group consisting of SEQ ID NOs:1-235;
(c) immortalizing the cell isolated in step (b); and
(d) isolating antibodies from the immortalized cell.

26-27. (Canceled)

28. A humanized antibody which specifically binds to a domain of a polypeptide comprising an amino acid sequence selected from the group consisting of SEQ ID NOs: 1-235.

29-35. (Canceled)

36. A method for determining the presence of a protein comprising exposing a sample suspected of containing the protein to the humanized antibody of claim 28 and determining binding of said antibody to the sample.

37. A kit comprising the humanized antibody of claim 28 and instructions for using the humanized antibody to detect a protein that binds to the antibody.

38. An isolated nucleic acid encoding the humanized antibody of claim 28.

39. A method for modulating the activity of the polypeptide of claim 10, the method comprising contacting the polypeptide with a compound that binds to the polypeptide in a concentration sufficient to modulate the activity of the polypeptide.

40. The method of claim 39, wherein the compound that binds the polypeptide is an antibody that selectively binds a polypeptide consisting of an amino acid sequence selected for the group consisting of SEQ ID NOs: 1-235.

41. A method of treating a disorder in a mammal, the method comprising:
(a) identifying a mammal with the disorder; and
(b) administering to the mammal a compound that modulates the expression or activity of the polypeptide of claim 10,
wherein the administration results in an amelioration of one or more symptoms of the disorder.

42. A method for detecting the presence of a polypeptide of claim 10 in a sample, the method comprising:
contacting the sample with a compound that selectively binds to a polypeptide consisting of an amino acid sequence selected from the group consisting of SEQ ID NOs: 1-235; and
determining whether the compound binds to the polypeptide in the sample.

43. A method for detecting the presence of a disorder in a mammal, the method comprising:
(a) providing a biological sample derived from the mammal;
(b) contacting the sample with a compound that binds to the polypeptide of claim 17 or to a nucleic acid that encodes the polypeptide of claim 17; and
(c) determining whether the compound binds to the sample,
wherein binding of the compound to the sample indicates the presence or absence of the disorder in the mammal.

44. A method for imaging a site in a mammal, the method comprising:
(a) administering a compound to a mammal, wherein the compound binds to the polypeptide of claim 10 or to a nucleic acid that encodes the polypeptide; and
(b) detecting the compound with an imaging detector, thereby imaging the site in the mammal.

45. A method for identifying a compound that modulates the activity of the polypeptide of claim 10, the method comprising:
(a) contacting the polypeptide of claim 10 with a test compound; and
(b) determining the effect of the test compound on the activity of the polypeptide, to thereby identify a compound that modulates the activity of the polypeptide.

46. A method for identifying a compound that modulates the expression of the nucleic acid of claim 2, the method comprising:
contacting the nucleic acid of claim 2 with a test compound; and
determining the effect of the test compound on the expression of the nucleic acid, to thereby identify a compound that modulates the expression of the nucleic acid.

47. A polypeptide profile that is characteristic of a given cell, wherein the profile comprises a representation of at least ten different polypeptides in the cell, wherein each of the at least ten different polypeptides comprises an amino acid sequence selected from the group consisting of SEQ ID NOs: 1-235, and wherein the polypeptide profile is a reproducible characteristic of the cell.

48-54. (Canceled)

55. A database, stored on a machine-readable medium, comprising:
(a) two categories of data respectively representing: (i) peptide profiles and (ii) cell sources; and
(b) associations among instances of the two categories of data,
wherein the data representing polypeptide profiles comprise the peptide profile of claim 46, and
wherein the database configures a computer to enable finding instances of data of one of the categories based on their associations with instances of data the other category.

56. A method of selecting an antibody, the method comprising:
(a) contacting the polypeptide of claim 10 with an in vitro library of antibodies;
(b) binding an antibody to the polypeptide; and
(c) selecting the antibody that binds to the polypeptide.

57. An immunogenic composition comprising a polypeptide comprising an amino acid sequence selected from the group consisting of SEQ ID NOs: 1-235, the composition when injected into a mammal eliciting an immunogenic response directed against a polypeptide consisting of an amino acid sequence selected from the group consisting of SEQ ID NOs: 1-235.

58. A method for treating a cancer comprising administering to a patient an amount of a composition comprising a polypeptide comprising an amino acid sequence selected from the group consisting of SEQ ID NOs: 1-235 in an amount sufficient to elicit an immunogenic response.

59. A method for treating a cancer patient, the method comprising administering to the patient an antibody that selectively binds to a peptide consisting of an amino acid sequence selected from the group consisting of SEQ ID NOs: 1-235.

60. A peptide array comprising at least 100 peptides selected from the group consisting of peptides consisting of an amino acid sequence selected from the group consisting of SEQ ID NOs:1-235, each peptide linked to a solid support at a known location.

61. A collection of at least 10 polypeptide arrays, each array comprising at least 100 polypeptides consisting of an amino acid sequence selected from the group consisting of SEQ ID NOs: 1-235, each peptide linked to a solid support at a known location.

62. A method for identifying a compound that binds to a naturally processed class I MHC-binding polypeptide, the method comprising exposing a test compound to a collection of at least 100 polypeptides selected from the group consisting of polypeptides having an amino acid sequence selected from the group consisting of SEQ ID NOs: 1-235, and identifying a peptide to which the test compound binds.

63. A method for identifying a compound that binds to a naturally processed class II MHC-binding polypeptide, the method comprising exposing a test compound to a collection of at least 100 polypeptides selected from the group consisting of polypeptides having an amino acid sequence selected from the group consisting of SEQ ID NOs: 1-235 and identifying a peptide to which the test compound binds.

64. A database, stored on a machine-readable medium, comprising:
(a) three categories of data respectively representing (i) polypeptides, (ii) cell sources, and (iii) cell treatments; and
(b) associations among instances of the three categories of data,
wherein the data representing peptides comprises at least 100 polypeptides each having an amino acid sequence selected from the group consisting of SEQ ID NOs: 1-235, and
wherein the database configures a computer to enable finding instances of data of one of the categories based on their associations with instances of data of at least one other category.

65. A polypeptide profile that is characteristic of a selected cell under selected conditions, wherein the profile comprises a representation of at least ten different polypeptides expressed by the cell, wherein each of the at least ten different polypeptides comprising an amino acid sequence selected from the group consisting of SEQ ID NOs:1-235, and wherein the polypeptide profile is a reproducible characteristic of the cell.
